(19) United States
(12) Patent Application Publication
(10) Pub. No.: US 2007/0117769 A1
(43) Pub. Date: May 24, 2007

(54) Methods of modulating cell cycle and cell signaling pathways using biliverdin reductase

(76) Inventor: Mahin D. Maines, Rochester, NY (US)

Correspondence Address: NIXON PEABODY LLP - PATENT GROUP, CLINTON SQUARE, P.O. BOX 31051, ROCHESTER, NY, 14603-1051, US

(21) Appl. No.: 10/584,886

(22) PCT Filed: Sep. 29, 2004
(86) PCT No.: PCT/US04/31866

§ 371 Date: Aug. 31, 2006

Related U.S. Application Data
(60) Provisional application No. 60/506,805, filed on Sep. 29, 2003.
(60) Provisional application No. 60/539,006, filed on Jan. 23, 2004.

(51) Int. Cl. A61K 48/00; C07H 21/04; C12N 9/06

(52) U.S. Cl. 514/44; 435/455; 435/325; 435/320.1; 536/23.2; 435/191

(57) ABSTRACT

The present invention relates to a method of modifying expression of cell cycle or cell signaling proteins. This method involves modifying the nuclear or cellular concentration of biliverdin reductase, or fragments or variants thereof, in a cell, whereby a change in the nuclear or cellular concentration of biliverdin reductase, or fragments or variants thereof, modifies the transcription or expression of cell cycle or cell signaling proteins.

This application claims the priority benefit of Provisional U.S. Patent Applications Ser. No. 60/506,805, filed Sep. 29, 2003, and 60/539,006, filed Jan. 23, 2004, each of which is hereby incorporated by reference in its entirety.

The present invention was made, at least in part, with funding received from the National Institutes of Health under grant ES04066. The U.S. government may have certain rights in this invention.

### FIELD OF THE INVENTION

The present invention relates generally to the use of biliverdin reductase, or functional fragments or variants thereof, to modify the expression of cell cycle and cell signaling pathways.

### BACKGROUND OF THE INVENTION

The well-known function of biliverdin IXα reductase (“BVR”) is catalysis of biliverdin reduction to bilirubin (Singleton et al., J. Biol. Chem. 240:4780-4789 (1965); O'Carra et al., J. Biochem. 125:110P (1971); Kutty et al., J. Biol. Chem. 256:3956-3962 (1981); Fakhrai et al., J. Biol. Chem. 267:4023-4029 (1992); Maines et al., Arch. Biochem. Biophys. 300:320-326 (1993); Maines et al., Eur. J. Biochem. 235:372-381 (1996); Ennis et al., Biochem. J. 328:33-36 (1997); McDonagh, Nat. Struct. Biol. 8:198-200 (2001)). Biliverdin is a product of cleavage of the heme macrocycle (Fe-protoporphyrin IX, hemin, heme b) by the microsomal enzyme heme oxygenase (HO) system (reviewed in Tenhunen et al., J. Biol. Chem. 244:6388-6394 (1969); Maines, “HEME OXYGENASE: Clinical Application and Functions,” CRC Press Inc., Boca Raton, Fla. 1-296 (1992); Wilks et al., Biochemistry 35:930-936 (1996)). The enzymes are also known as the heat shock protein-32 (HSP32) family of proteins (Shibahara et al., J. Biol. Chem. 262:12884-12892 (1987); Keyse et al., Proc. Natl. Acad. Sci. USA 86:99-103 (1989); Agarwal et al., Amer. J. Physiol. 27:F814-F823 (1996); Gong et al., Antiox. Redox. Signal. 4:249-257 (2002); Shibahara et al., Exper. Biol. Med. 228:472-473 (2003)).

Recent studies have uncovered features of the reductase that are unrelated to its reductase activity; BVR has been characterized as a serine/threonine kinase (Salim et al., J. Biol. Chem. 276:10929-10934 (2001)) that is activated by oxygen radicals and translocates into the nucleus in response to cGMP and oxidative stress (Maines et al., J. Pharmacol. Exper. Ther. 296:1091-1097 (2001); Ahmad et al., J. Biol. Chem. 277:9226-9232 (2002)). The presence of a “leucine zipper” motif: Leu¹²⁹×6 Leu¹³⁶×6 Lys¹⁴³×6 Leu¹⁵⁰×6 Leu¹⁵⁷, that is preceded by a conserved basi domain, together with mutation analyses, identified the sequence as a dimerization domain. The DNA binding ability of the protein was confirmed by demonstrating its binding to the consensus sequence of AP-1 sites in the HO-1 promoter. The crystal structure of rat BVR has been solved (Kikuchi et al., Nat. Struct. Biol. 8:221-228 (2001); Whitby et al., J. Mol. Biol. 319:1199-1210 (2002)); using coordinates for rat enzyme, in the predicted 3-dimensional structure of human reductase, a dimerization domain was found. This is consistent with the ability of BVR to bind DNA. Collectively, these criteria support the inclusion of BVR as a member of the bZip DNA binding family of transcription factors. The members of the family, which include transcription factors such Myc, GCN₄, c-Jun, CREB, c-Fos, sYAP, and ATF-2 (VanStraaten, Proc. Natl. Acad. Sci. USA 80:3183-3187 (1983); Gazin et al., EMBO J. 3:383-387 (1984); Hinnebusch, Proc. Natl. Acad. Sci. USA 81:6442-6446 (1984); Bolmann et al., Science 238:1386-1392 (1987); Hoeffler et al., Science 242:1430-1433 (1988); Moye-Rowley et al., Genes Dev. 3:283-292 (1989); Grupe et al., EMBO J. 9:1749-1756 (1990); Morookah et al., J. Biol. Chem. 270:30084-30092 (1995)), activate cell signaling pathways, including the MAPK pathway, for proliferation, differentiation, survival, and apoptosis. HO-1 is among those genes whose expression can be upregulated by activation of the MAPK pathway. The current understanding among investigators is that upregulation of HO-1 is associated with an enhanced defense mechanism against stress (Otterbein et al., Nat. Med. 6:422-428 (2000); Motterlini et al., Free Rad. Biol. Med. 28:1303-1312 (2000); Morse et al., Grit. Care. Med. 30:S12-S17 (2002); Zhang et al., J. Biol Chem. 278:22061-22070 (2003); Chen et al., Exper. Biol. Med. 228:447-453 (2003); Otterbein et al., Trends Immunol. 24:449-455 (2003); Maines, Tox. Sci. 71:9-10 (2003)). Moreover, recently the product of HO activity, biliverdin, has been shown to play an essential role in the earliest stages of embryogenesis to mandate dorsal axis formation in Xenopus embryo (Falchuk et al., Proc. Natl. Acad. Sci. 99:251-256 (2002)).

HO-1 stress response is mediated by AP-1 binding to multiple copies of consensus sequence TGACTCA (Alam et al., J. Biol. Crew. 267:21894-21900 (1992)). AP-1 family of proteins form homo- or hetero-dimers that include, among others, c-Jun/ATF-2 hetero-dimer, which binds to both the AP-1 site and the CRE site (TGACN TCA). In addition to HO-1, AP-1 sites are found in many promoters of genes, including growth factors, chemokines, and cytokines. ATF-2 as a homodimer binds to CRE, whereas hetero-dimerization of ATF-2 with c-Jun increases its affinity for AP-1 by about 4-fold over that of the c-Jun/c-Fos hetero-dimer with an increase in association time of the complex with DNA (Benbrook et al., Oncogene 5:295-302 (1990); Herr et al., Carcinogenesis 15:1105-1113 (1994)). HO-1 is also induced by cAMP and CRE activation (Immenschuh et al., Biochem J. 334:141-146 (1998)).

The structural and activity profile of the reductase in vitro are consistent with its having a regulatory role in cellular functions. However, it remains to be understood whether criteria ascribed to BVR do in fact have biological significance, such as whether increased expression of BVR in the cell effects regulation of gene expression for cell cycle or cell signaling proteins.

The present invention is directed to overcoming these and other limitations in the art.

### SUMMARY OF THE INVENTION

The present invention relates to a method of modifying expression of cell cycle or cell signaling proteins. This method involves modifying the nuclear or cellular concentration of biliverdin reductase, or fragments or variants thereof, in a cell, whereby a change in the nuclear or cellular concentration of biliverdin reductase, or fragments or variants thereof, modifies the transcription or expression of cell cycle or cell signaling proteins.

A further aspect of the present invention relates to methods of treating conditions associated with expression levels of cell cycle or cell signaling proteins, by modifying the nuclear or cellular concentration of biliverdin reductase, or fragments or variants thereof, in a cell, in accordance with the first aspect of the present invention, and thereby altering the transcription of a cell cycle or cell signaling protein to treat a condition associated therewith. In particular, up- or down-regulation of cell cycle or cell signaling protein expression can be effected by the present invention.

Yet another aspect of the present invention relates to an siRNA molecule capable of inhibiting expression of BVR, as well as DNA molecules and constructs responsible for producing the siRNA molecule, and in vivo and ex vivo host cells containing the same.

In the present invention, it is shown that in cells infected with adenoviral BVR expression vectors, designated Ad-bBVR, induction of hBVR gene expression results in increased levels of transcription and expression for a number of cell cycle and signaling proteins, including ATF-2 and HO-1. As demonstrated hereinafter, the BVR amino acid sequence is highly conserved, particularly among mammals. The evolutionarily conserved functional and structural features include the bZip motif, the kinase activity, and having dual pH/cofactor requirements (Ennis et al., Biochem. J. 328:33-36 (1997); Huang et al., J. Biol. Chem. 264:7844-7849 (1989), which are hereby incorporated by reference in their entirety). Therefore, findings with human enzyme predictably may be also applicable to other mammalian species. The present invention identifies BVR as a novel regulator of ATF-2 and HO-1 expression and suggests that modifying expression of BVR should be a useful approach to change the expression profile of a host of genes in the cell, including those encoding various cell cycling or cell signaling proteins that are involved in cellular process such as, inter alia, cell proliferation, differentiation, survival, and apoptosis.

### BRIEF DESCRIPTION OF THE DRAWINGS

FIG. 1 schematically illustrates hBVR adenovirus constructs. Abbreviations: AdX=recombinant Adeno-X virus; TRE=Tet-responsive element; PₘᵢₙCMV=minimal immediate early promoter of cytomegalovirus; GFP=gene GFP; FLAG=M₂ flag sequence; hBVR=human BVR gene; VP16=C-terminal activation domain of Herpes simplex virus VP16 protein; rtetR=“reverse” mutant of Tet repressor. The large block arrows represent the direction of translation of ORF “EGFP/FLAG/BVLRA”. The small arrows represent the direction of transcription from PₘᵢₙCMV.

FIGS. 2A-C present analyses of biliverdin reductase message and activity of 293A cells infected with adenovirus hBVR expression system. 293A cells were infected with wild-type Ad-EGFP-FLAG-hBVR (Ad-hBVR, forward arrow) (lane 4), Ad-INV-hBVR (Ad-hBVR, reverse arrow) (lane 3), or Adeno-X Tet-On (Adx) (lane 2). 293A cells were used as the control (lane 1). Northern blot analysis, using GAPDH as the loading control was carried out as described infra 24 h after infection. 5 μg RNA was used in each lane. BVR activity was measured at pH 6.7 with NADH as the cofactor by measuring the rate of conversion of biliverdin to bilirubin (Fakbrai et al., J. Biol. Chem. 267:4023-4029 (1992), which is hereby incorporated by reference in its entirety).

FIGS. 3A-E demonstrate time course of induction of hBVR in response to doxycycline addition. 293A cells transfected with wild-type hBVR were analyzed for expression of the gene at the indicated time points. For protein visualization, rabbit polyclonal antihuman BVR antibody (FIG. 3A) or monoclonal anti-FLAG (FIG. 3B) was used. Activity was measured as described infra (FIG. 3C). Green fluorescence of the cells was captured 24 h after addition of doxycycline (FIG. 3D). FIG. 3E shows the cells under visible light.

FIGS. 4A-D are photographs showing increased expression of ATF-2 mRNA and protein in cells infected with Ad-hBVR. Native and phosphorylated ATF-2 in Ad-hBVR infected cells. 293A cells were infected with Ad-hBVR construct and analyzed for the expression of ATF-2 mRNA and protein. A 1,600 bp ATF-2 probe was used for the Northern analysis; monoclonal antibodies to native or phosphorylated ATF-2 and ECL system were used for Western blot analyses.

FIGS. 5A-B show hBVR binding to ATF-2 promoter. AP-1 binding assay was carried out using either in vitro translated hBVR by a TNT protein translation system or nuclear extract from 293 cells transfected with Ad-BVR expression vector. DNA fragment upstream of ATF-2 ATG codon shown in Table 3 was used as a binding probe. Super shift assay was carried out using polyclonal antibody to hBVR. FIG. 5A: Lanes: 1=translation system+³²P-labeled DNA probe without hBVR; 2,4 & 5=translated hBVR+labeled DNA; 3=translated hBVR+labeled DNA+10-fold excess of unlabeled DNA; 6=translated hBVR+labeled DNA+antibody to hBVR. FIG. 5B: Lanes: 1, 2, and 3:=³²P-labeled AP-1 DNA probe+nuclear extracts isolated from 239 cells transfected with Ad-BVR at times 0, 6, and 24 hours respectively after induction expression of hBVR. Lane 4: ³²P-labeled AP-1 DNA probe+nuclear extracts isolated from 239 cells transfected with Ad-BVR at time 24 hours after induction expression of hBVR+antibody to human BVR.

FIGS. 6A-B illustrate that hBVR binds to ATF/CRE consensus sequence. The assay was carried out using hBVR translated in vitro with a TNT protein translation system and nuclear extracts from 293 cells transfected with Ad-BVR expression vector, ³²labeled DNA fragments containing 4 or 1 ATF/CRE sites were used in the gel shift binding experiment (Table 3). For competition analysis, unlabeled CRE containing oligo nucleotide was added at 2×, 5× and 10× excess that of labeled CRE oligo. FIG. 6A: from left, lane: 1=translation system without hBVR; 2=translated hBVR+labeled CRE oligo; 3, 4 & 5=translated hBVR+unlabeled CRE oligo nucleotide at 2×, 5× and 10× excess of that of labeled CRE, respectively; 6=translated ATF-2+labeled CRE oligo as a positive control for CRE binding. FIG. 6B, from left lane 1, 2, and 3. FIG. 6b: Lanes: 1, 2, and 3:=³² P4abeled CRE DNA probe+nuclear extracts isolated from 239 cells transfected with Ad-BVR at times 0, 6, and 24 hours respectively after induction expression of hBVR. Lane 4: ³²P-labeled AP-1 DNA probe+nuclear extracts at time point 24 hours after induction expression of hBVR+antibody to human BVR.

FIGS. 7A-C illustrate transcriptional activation of ATF-2 and c-jun reporters by BVR. 293 cells were transfected with ATF-2 (pGL3/ATF2, 0.4 μg), or with AP1-responsive c-jun luciferase (pGL3/c-jun, 0.4 μg) reporters. The controls consisted of BVR expression vector (pcDNA3/1BVR, 0.4 μg) alone or with basic luciferase vector (pGL3, 0.4 μg), or both promoter reporters (pGL3/ATF-2 or pGL3/c-jun) cotransfected with empty pcDNA3, or c-jun promoter mutant (pGL3/c-jun^{MT}) cotransfected with pcDNA3/hBVR. pCMVβ-gal (0.4 μg) vector was also included under all conditions for normalization of transfection. The usage of each construct is indicated in FIG. 7A and FIG. 7B. In FIG. 7A, cells transfected with ATF-2 reporter were cotransfetcted with pcDNA3 vector expressing hBVR. In FIG. 7B, cells transfected with c-jun promoter (pGL3/c-jun) were cotransfected with pcDNA3/hBVR. After 48 h, cells were lysed and assayed for luciferase and β-gal activities. Luciferase activity normalized for transfection efficiency against β-gal activity, is presented as the mean ±S.D. of the fold activation when compared with that of either vector pGL3/ATF-2 alone (a), or vector pGL3/c-jun alone (b), which were assigned the value 1. The results are representative of two experiments with quadruplicate wells.

FIG. 8 schematically illustrates HO-1 gene expression induced in cells infected with Ad-hBVR. 293A cells were infected with Ad-hBVR and gene expression was induced by the addition of doxycycline (Dox). Northern blot analysis of HO-1 mRNA used a full length HO-1 cDNA probe and HO-1 protein levels were measured by ELISA. Cells infected with Ad-INV-hBVR did not show increased expression of HO-1. The amount of hHO-1 was compared to 0 time point taken as 100%. The values are expressed as a mean ±s.d. of three separate experiments.

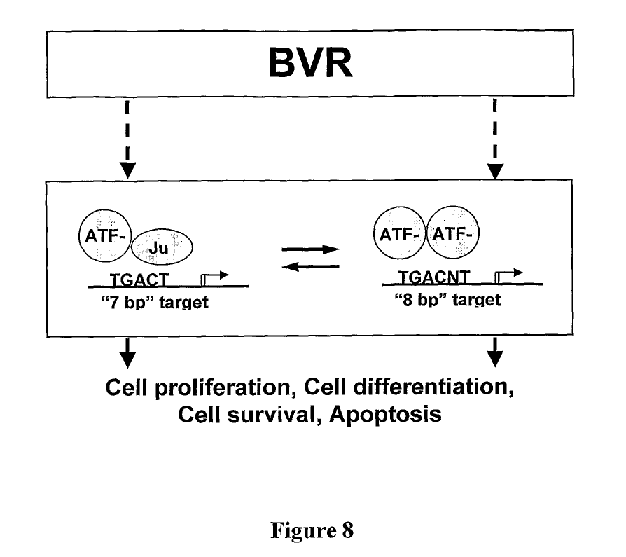

### DETAILED DESCRIPTION OF THE INVENTION

The present invention relates to the use of biliverdin reductase (“BVR!”) to regulate expression of cell cycle and cell signaling pathways. As a consequence, by modifying the nuclear or cellular concentration of BVR, or fragments or variants thereof, the expression of cell cycle and cell signaling proteins can be regulated, i.e., either enhanced or suppressed.

Consequently, a further aspect of the present invention relates to methods of treating conditions associated with expression levels of cell cycle or cell signaling proteins. By modifying the nuclear or cellular concentration of BVR, or fragments or variants thereof, in a cell, in accordance with the present invention, the expression level of a cell cycle or cell signaling protein can be altered to treat a condition associated with its over- or under-expression. For instance, conditions associated with under-expression can be alleviated by modifying the nuclear or cellular concentration of BVR, or fragments or variants thereof, in a cell so as to enhance the expression level of the cell cycle or cell signaling protein associated with the condition. Likewise, conditions associated with over-expression can be alleviated by modifying the nuclear or cellular concentration of BVR, or fragments or variants thereof, in a cell so as to suppress the expression level of the cell cycle or cell signaling protein associated with the condition.

To increase the nuclear or cellular concentration of BVR, or fragments or variants thereof, either BVR or the fragments or variants thereof can be introduced into the cell directly or expressed therein via in vivo cell transformation. To decrease the nuclear concentration of BVR, antisense BVR RNA can be introduced into the cell directly or expressed therein via in vivo transformation, which antisense BVR RNA inhibits BVR mRNA translation. Alternatively, short interfering RNA molecules (siRNAs) that target (or bind to) BVR may be introduced into the cell directly or expressed therein via in vivo transformation to inhibit BVR expression/activity. Thus, both protein or RNA delivery systems or gene delivery systems can be employed in the present invention.

As used herein, the terms biliverdin reductase and BVR refer to any mammalian BVR, but preferably human BVR (“hBVR”).

One form of hBVR has an amino acid sequence corresponding to SEQ ID NO: 1 as follows:

```
Met Asn Ala Glu Pro Glu Arg Lys Phe Gly Val Val Val Val Gly Val
    1 5 10 15
Gly Arg Ala Gly Ser Val Arg Met Arg Asp Leu Arg Asn Pro His Pro
                          20 25 30
Ser Ser Ala Phe Leu Asn Leu Ile Gly Phe Val Ser Arg Arg Glu Leu
                  35 40 45
Gly Ser Ile Asp Gly Val Gln Gln Ile Ser Leu Glu Asp Ala Leu Ser
          50 55 60
Ser Gln Glu Val Glu Val Ala Tyr Ile Cys Ser Glu Ser Ser Ser His
  65 70 75 80
Glu Asp Tyr Ile Arg Gln Phe Leu Asn Ala Gly Lys His Val Leu Val
                                  85 90 95
Glu Tyr Pro Met Thr Leu Ser Leu Ala Ala Ala Gln Glu Leu Trp Glu
                        100 105 110
Leu Ala Glu Gln Lys Gly Lys Val Leu His Glu Glu His Val Glu Leu
                115 120 125
Leu Met Glu Glu Phe Ala Phe Leu Lys Lys Glu Val Val Gly Lys Asp
        130 135 140
Leu Leu Lys Gly Ser Leu Leu Phe Thr Ser Asp Pro Leu Glu Glu Asp
145 150 155 160
Arg Phe Gly Phe Pro Ala Phe Ser Gly Ile Ser Arg Leu Thr Trp Leu
                                165 170 175
Val Ser Leu Phe Gly Glu Leu Ser Leu Val Ser Ala Thr Leu Glu Glu
                        180 185 190
Arg Lys Glu Asp Gln Tyr Met Lys Met Thr Val Cys Leu Glu Thr Glu
                195 200 205
Lys Lys Ser Pro Leu Ser Trp Ile Glu Glu Lys Gly Pro Gly Leu Lys
        210 215 220
Arg Asn Arg Tyr Leu Ser Phe His Phe Lys Ser Gly Ser Leu Glu Asn
225 230 235 240
Val Pro Asn Val Gly Val Asn Lys Asn Ile Phe Leu Lys Asp Gln Asn
                                245 250 255
Ile Phe Val Gln Lys Leu Leu Gly Gln Phe Ser Glu Lys Glu Leu Ala
                        260 265 270
Ala Glu Lys Lys Arg Ile Leu His Cys Leu Gly Leu Ala Glu Glu Ile
                275 280 285
Gln Lys Tyr Cys Cys Ser Arg Lys
        290 295
```

Heterologous expression and isolation of hBVR is described in Maines et al., Eur. J. Biochem. 235:372-381 (1996); Maines et al., Arch. Biochem. Biophys. 300:320-326 (1993), which are hereby incorporated by reference in their entirety. A DNA molecule encoding this form of hBVR has a nucleotide sequence corresponding to SEQ ID NO: 2 as follows:

ggggtggcgc ccggagctgc acggagagcg tgcccgtcag tgaccgaaga agagaccaag 60
atgaatgcag agcccgagag gaagtttggc gtggtggtgg ttggtgttgg ccgagccggc 120
tccgtgcgga tgagggactt gcggaatcca cacccttcct cagcgttcct gaacctgatt 180
ggcttcgtgt cgagaaggga gctcgggagc attgatggag tccagcagat ttctttggag 240
gatgctcttt ccagccaaga ggtggaggtc gcctatatct gcagtgagag ctccagccat 300
gaggactaca tcaggcagtt ccttaatgct ggcaagcacg tccttgtgga ataccccatg 360
acactgtcat tggcggccgc tcaggaactg tgggagctgg ctgagcagaa aggaaaagtc 420
ttgcacgagg agcatgttga actcttgatg gaggaattcg ctttcctgaa aaaagaagtg 480
gtggggaaag acctgctgaa agggtcgctc ctcttcacat ctgacccgtt ggaagaagac 540
cggtttggct tccctgcatt cagcggcatc tctcgactga cctggctggt ctccctcttt 600
ggggagcttt ctcttgtgtc tgccactttg gaagagcgaa aggaagatca gtatatgaaa 660
atgacagtgt gtctggagac agagaagaaa agtccactgt catggattga agaaaaagga 720
cctggtctaa aacgaaacag atatttaagc ttccatttca agtctgggtc cttggagaat 780
gtgccaaatg taggagtgaa taagaacata tttctgaaag atcaaaatat atttgtccag 840
aaactcttgg gccagttctc tgagaaggaa ctggctgctg aaaagaaacg catcctgcac 900
tgcctggggc ttgcagaaga aatccagaaa tattgctgtt caaggaagta agaggaggag 960
gtgatgtagc acttccaaga tggcaccagc atttggttct tctcaagagt tgaccattat 1020
ctctattctt aaaattaaac atgttgggga aacaaaaaaa aaaaaaaaaa 1070

The open reading frame which encodes hBVR of SEQ ID NO: 1 extends from nt 1 to nt 888.

Another form of hBVR has an amino acid sequence according to SEQ ID NO: 3 as follows:

```
Met Asn Thr Glu Pro Glu Arg Lys Phe Gly Val Val Val Val Gly Val
    1 5 10 15
Gly Arg Ala Gly Ser Val Arg Met Arg Asp Leu Arg Asn Pro His Pro
                          20 25 30
Ser Ser Ala Phe Leu Asn Leu Ile Gly Phe Val Ser Arg Arg Gln Leu
                  35 40 45
Gly Ser Ile Asp Gly Val Gln Gln Ile Ser Leu Gln Asp Ala Leu Ser
          50 55 60
Ser Gln Glu Val Glu Val Ala Tyr Ile Cys Ser Glu Ser Ser Ser His
  65 70 75 80
Glu Asp Tyr Ile Arg Gln Phe Leu Asn Ala Gly Lys His Val Leu Val
                                  85 90 95
Glu Tyr Pro Met Thr Leu Ser Leu Ala Ala Ala Gln Gln Leu Trp Gln
                        100 105 110
Leu Ala Gln Gln Lys Gly Lys Val Leu His Gln Gln His Val Gln Leu
                115 120 125
Leu Met Gln Gln Phe Ala Phe Leu Lys Lys Gln Val Val Gly Lys Asp
        130 135 140
Leu Leu Lys Gly Ser Leu Leu Phe Thr Ala Gly Pro Leu Gln Gln Gln
145 150 155 160
Arg Phe Gly Phe Pro Ala Phe Ser Gly Ile Ser Arg Leu Thr Trp Leu
                                165 170 175
Val Ser Leu Phe Gly Gln Leu Ser Leu Val Ser Ala Thr Leu Gln Gln
                        180 185 190
Arg Lys Gln Asp Gln Tyr Met Lys Met Thr Val Cys Leu Gln Thr Gln
                195 200 205
Lys Lys Ser Pro Leu Ser Trp Ile Gln Gln Lys Gly Pro Gly Leu Lys
        210 215 220
Arg Asn Arg Tyr Leu Ser Phe His Phe Lys Ser Gly Ser Leu Glu Asn
225 230 235 240
Val Pro Asn Val Gly Val Asn Lys Asn Ile Phe Leu Lys Asp Gln Asn
                                245 250 255
Ile Phe Val Gln Lys Leu Leu Gly Gln Phe Ser Glu Lys Glu Leu Ala
                        260 265 270
Ala Glu Lys Lys Arg Ile Leu His Cys Leu Gly Leu Ala Glu Glu Ile
                275 280 285
Gln Lys Tyr Cys Cys Ser Arg Lys
        290 295
```

This hBVR sequence is reported at Komuro et al., NCBI Accession No. G02066, direct submission to the EMBL Data Library (1998), which is hereby incorporated by reference in its entirety. Differences between the hBVR of SEQ ID NO: 1 and the hBVR of SEQ ID NO: 3 are at aa residues 3, 154, 155, and 160. Thus, residue 3 can be either alanine or threonine, residue 154 can be either alanine or serine, residue 155 can be either aspartic acid or glycine, and residue 160 can be either aspartic acid or glutamic acid.

In addition, BVR from other mammals, such as rat (rBVR), have been recombinantly expressed and isolated (Fakhrai et al., J. Biol. Chem. 267:4023-4029 (1992), which is hereby incorporated by reference in its entirety). One form of rat biliverdin reductase (“rBVR”) has an amino acid sequence corresponding to SEQ ID NO: 4 as follows:

```
Met Asp Ala Glu Pro Lys Arg Lys Phe Gly Val Val Val Val Gly Val
    1 5 10 15
Gly Arg Ala Gly Ser Val Arg Leu Arg Asp Leu Lys Asp Pro Arg Ser
                          20 25 30
Ala Ala Phe Leu Asn Leu Ile Gly Phe Val Ser Arg Arg Glu Leu Gly
                  35 40 45
Ser Leu Asp Glu Val Arg Gln Ile Ser Leu Glu Asp Ala Leu Arg Ser
          50 55 60
Gln Glu Ile Asp Val Ala Tyr Ile Cys Ser Glu Ser Ser Ser His Glu
  65 70 75 80
Asp Tyr Ile Arg Gln Phe Leu Gln Ala Gly Lys His Val Leu Val Glu
                                  85 90 95
Tyr Pro Met Thr Leu Ser Phe Ala Ala Ala Gln Gln Leu Trp Gln Leu
                        100 105 110
Ala Ala Gln Lys Gly Arg Val Leu His Gln Glu His Val Gln Leu Leu
                115 120 125
Met Glu Gln Phe Glu Phe Leu Arg Arg Gln Val Leu Gly Lys Gln Leu
        130 135 140
Leu Lys Gly Ser Leu Arg Phe Thr Ala Ser Pro Leu Gln Glu Gln Arg
145 150 155 160
Phe Gly Phe Pro Ala Phe Ser Gly Ile Ser Arg Leu Thr Trp Leu Val
                                165 170 175
Ser Leu Phe Gly Gln Leu Ser Leu Ile Ser Ala Thr Leu Glu Gln Arg
                        180 185 190
Lys Gln Asp Gln Tyr Met Lys Met Thr Val Gln Leu Gln Thr Gln Asn
                195 200 205
Lys Gly Leu Leu Ser Trp Ile Glu Gln Lys Gly Pro Gly Leu Lys Arg
        210 215 220
Asn Arg Tyr Val Asn Phe Gln Phe Thr Ser Gly Ser Leu Glu Gln Val
225 230 235 240
Pro Ser Val Gly Val Asn Lys Asn Ile Phe Leu Lys Asp Gln Asp Ile
                                245 250 255
Phe Val Gln Lys Leu Leu Asp Gln Val Ser Ala Glu Asp Leu Ala Ala
                        260 265 270
Gln Lys Lys Arg Ile Met His Cys Leu Gly Leu Ala Ser Asp Ile Gln
                275 280 285
Lys Leu Cys His Gln Lys Lys
        290 295
```

Heterologous expression and isolation of rBVR is described in Fakhrai et al., J. Biol. Chem. 267(6):4023-4029 (1992), which is hereby incorporated by reference. The rBVR of SEQ. ID. No. 4 shares about 82% aa identity to the hBVR of SEQ. ID. No. 1, with variations in aa residues being highly conserved. The DNA molecule encoding this form of rBVR has a nucleotide sequence corresponding to SEQ ID NO: 5 as follows:

ggtcaacagc taagtgaagc catatccata gagagtttgt gccagtgccc caagatcctg 60
aacctctgtc tgtcttcgga cactgactga agagaccgag atggatgccg agccaaagag 120
gaaatttgga gtggtagtgg ttggtgttgg cagagctggc tcggtgaggc tgagggactt 180
gaaggatcca cgctctgcag cattcctgaa cctgattgga tttgtgtcca gacgagagct 240
tgggagcctt gatgaagtac ggcagatttc tttggaagat gctctccgaa gccaagagat 300
tgatgtcgcc tatatttgca gtgagagttc cagccatgaa gactatatac ggcagtttct 360
gcaggctggc aagcatgtcc tcgtggaata ccccatgaca ctgtcatttg cggcggccca 420
ggagctgtgg gagctggccg cacagaaagg gagagtcctg catgaggagc acgtggaact 480
cttgatggag gaattcgaat tcctgagaag agaagtgttg gggaaagagc tactgaaagg 540
gtctcttcgc ttcacagcta gcccactgga agaagagaga tttggcttcc ctgcgttcag 600
cggcatttct cgcctgacct ggctggtctc cctcttcggg gagctttctc ttatttctgc 660
caccttggaa gagcgaaaag aggatcagta tatgaaaatg accgtgcagc tggagaccca 720
gaacaagggt ctgctgtcat ggattgaaga gaaagggcct ggcttaaaaa gaaacagata 780
tgtaaacttc cagttcactt ctgggtccct ggaggaagtg ccaagtgtag gggtcaataa 840
gaacattttc ctgaaagatc aggatatatt tgttcagaag ctcttagacc aggtctctgc 900
agaggacctg gctgctgaga agaagcgcat catgcattgc ctggggctgg ccagcgacat 960
ccagaagctt tgccaccaga agaagtgaag aggaagcttc agagacttct gaagggggcc 1020
agggtttggt cctatcaacc attcaccttt agctcttaca attaaacatg tcagataaac 1080
a 1081

The open reading frame which encodes rBVR of SEQ ID NO: 4 extends from nt 1 to nt 885.

A mouse BVR sequence is reprted at Genbank Accession NP_{—}080954, and has an amino acid sequence according to SEQ ID NO: 6 as follows:

```
Met Ser Thr Glu Pro Lys Arg Lys Phe Gly Val Val Val Val Gly Val
    1 5 10 15
Gly Arg Ala Gly Ser Val Arg Ile Arg Asp Ser Lys Asp Pro His Ser
                          20 25 30
Ser Ala Phe Leu Asn Leu Ile Gly Tyr Val Ser Arg Arg Gln Leu Gly
                  35 40 45
Ser Leu Asp Asn Val Arg Gln Ile Ser Leu Gln Asp Ala Leu Arg Ser
          50 55 60
Gln Glu Val Asp Val Ala Tyr Ile Cys Thr Glu Ser Ser Ser His Glu
  65 70 75 80
Asp Tyr Ile Arg Gln Phe Leu Gln Ala Gly Lys His Val Leu Val Glu
                                  85 90 95
Tyr Pro Met Ala Leu Ser Phe Ala Ala Ala Gln Gln Leu Trp Gln Leu
                        100 105 110
Ala Ala Gln Lys Gly Arg Val Leu His Glu Gln His Ile Gln Leu Leu
                115 120 125
Met Glu Gln Phe Gln Phe Leu Lys Arg Glu Val Ala Gly Lys Gln Leu
        130 135 140
Leu Lys Gly Ser Leu Arg Phe Thr Ala Ser Pro Leu Gln Gln Gln Lys
145 150 155 160
Phe Gly Phe Pro Ala Phe Ser Gly Ile Ser Arg Leu Thr Trp Leu Val
                                165 170 175
Ser Leu Phe Gly Glu Leu Ser Leu Ile Ser Ala Thr Met Glu Asn Arg
                        180 185 190
Lys Glu Asp Gln Tyr Met Lys Met Thr Val Gln Leu Gln Thr Gln Asn
                195 200 205
Lys Ser Pro Leu Ser Trp Ile Gln Gln Lys Gly Pro Gly Leu Lys Arg
        210 215 220
Asn Arg His Ile Ser Ile His Phe Lys Ser Gly Ser Leu Glu Gln Val
225 230 235 240
Pro Asn Val Gly Val Asn Lys Asn Ile Phe Leu Lys Asp Gln Asp Ile
                                245 250 255
Phe Ile Gln Lys Leu Leu Gly Gln Val Ser Ala Glu Asp Leu Ala Ala
                        260 265 270
Glu Lys Lys Arg Ile Leu His Cys Leu Gln Leu Ala Ser Asp Ile Gln
                275 280 285
Arg Leu Cys His Arg Lys Gln
        290 295
```

The mouse BVR sequence is about 81 percent identical to the human BVR sequence of SEQ ID NO: 1.

The pig BVR sequence has also been isolated, and has an amino acid sequence according to SEQ ID NO: 7 as follows:

```
Met Asn Ala Glu Pro Glu Arg Lys Phe Gly Val Val Val Val Gly Val
    1 5 10 15
Gly Arg Ala Gly Ser Val Arg Met Arg Asp Leu Arg Asn Pro His Pro
                          20 25 30
Ser Ser Ala Phe Leu Asn Leu Ile Gly Phe Val Ser Arg Arg Glu Leu
                  35 40 45
Gly Ser Ile Asp Gly Val Gln Gln Ile Ser Leu Glu Asp Ala Leu Ser
          50 55 60
Ser Gln Glu Val Glu Val Ala Tyr Ile Cys Ser Glu Ser Ser Ser His
  65 70 75 80
Glu Asp Tyr Ile Arg Gln Phe Leu Asn Ala Gly Lys His Val Leu Val
                                  85 90 95
Glu Tyr Pro Met Thr Leu Ser Leu Ala Ala Ala Gln Glu Leu Trp Glu
                        100 105 110
Leu Ala Glu Gln Lys Gly Lys Val Leu His Glu Glu His Val Glu Leu
                115 120 125
Leu Met Glu Glu Phe Ala Phe Leu Lys Lys Glu Val Val Gly Lys Asp
        130 135 140
Leu Leu Lys Gly Ser Leu Leu Phe Thr Ala Gly Pro Leu Glu Glu Glu
145 150 155 160
Arg Phe Gly Ser Pro Ala Phe Ser Gly Ile Ser Arg Leu Thr Trp Leu
                                165 170 175
Val Ser Leu Phe Gly Glu Leu Ser Leu Val Ser Ala Thr Leu Glu Glu
                        180 185 190
Arg Lys Glu Asp Gln Tyr Met Lys Met Thr Val Cys Leu Glu Thr Glu
                195 200 205
Lys Lys Ser Pro Leu Ser Trp Ile Glu Glu Lys Gly Pro Gly Leu Lys
        210 215 220
Arg Asn Arg Tyr Leu Ser Phe His Phe Lys Ser Gly Ser Leu Glu Asn
225 230 235 240
Val Pro Asn Val Gly Val Asn Lys Asn Ile Phe Leu Lys Asp Gln Asn
                                245 250 255
Ile Phe Val Gln Lys Leu Leu Gly Gln Phe Ser Glu Lys Glu Leu Ala
                        260 265 270
Ala Glu Lys Lys Arg Ile Leu His Cys Leu Gly Leu Ala Glu Glu Ile
                275 280 285
Gln Lys Tyr Cys Cys Ser Arg Lys
        290 295
```

The pig BVR sequence is about 98 percent identical to the human BVR sequence of SEQ ID NO: 1.

As described in greater detail in co-pending U.S. patent application Ser. No. 09/606,129 to Maines, filed Jun. 28, 2000 (which is hereby incorporated by reference in its entirety), BVR is characterized by an amazingly large number of functional domains and motifs, including without limitation: putative and/or demonstrated phosphorylation sites from aa 15 to 20, aa 21 to 23, aa 44 to 46 or 47, aa 49 to 54, aa 58 to 61, aa 64 to 67, aa 78 to 81, aa 79 to 82, aa 189 to 192, aa 207 to 209, aa 214 to 217, aa 222 to 227, aa 236 to 241, aa 245 to 250, aa 267 to 269 or 270, and aa 294 to 296 of SEQ ID NO: 1; a basic N-terminal domain characterized by aa 6 to 8 of SEQ ID NO: 1; a hydrophobic domain characterized by aa 9 to 14 of SEQ ID NO: 1; a nucleotide binding domain characterized by aa 15 to 20 of SEQ ID NO: 1; an oxidoreductase domain characterized by aa 90 to 97 of SEQ ID NO: 1; a leucine zipper spanning aa 129 to 157 of SEQ ID NO: 1; several kinase motifs, including aa 44 to 46, aa 147 to 149, and aa 162 to 164 of SEQ ID NO: 1; a nuclear localization signal spanning aa 222 to 228 of SEQ ID NO: 1; a myristylation site spanning aa 221 to 225 of SEQ ID NO: 1; a zinc finger domain spanning aa 280 to 293 of SEQ ID NO: 1; and several substrate binding domains. These domains are either identical or highly conserved among the above-identified mammalian BVR of SEQ ID NOS: 1, 3, 4, 6, and 7.

The use of homolgous BVR proteins or polypeptides (or their encoding DNA) from other mammals is also contemplated. Preferably, such homologous BVR proteins or polypeptides are characterized by an amino acid identity of at least about 70 percent, more preferably at least about 75 percent or 80 percent, most preferably at least about 85 percent or 90 percent or 95 percent as compared to the human BVR of SEQ ID NO: 1. Other mammalian cDNA molecules can be identified based upon their alignment with the human BVR cDNA of SEQ ID NO: 2, where such alignment preferably is at least about 60 percent identical; or by the ability of other mammalian BVR cDNA sequences to hybridize to the complement of SEQ ID NO: 2 under stringent hybridization and wash conditions. Exemplary stringent hybridization and wash conditions include, without limitation, hybridization at 50° C. or higher (i.e., 55° C., 600C, or 65° C.) in a hybridization medium that includes 0.9× (or higher, such as 2× or 5×) sodium citrate (“SSC”) buffer, followed by one or more washes at increasing stringency using 0.2×SSC buffer at temperatures from 42° C. up to the temperature of the hybridization step. Higher stringency can readily be attained by increasing the temperature for either hybridization or washing conditions or decreasing the sodium concentration of the hybridization or wash medium. Nonspecific binding may also be controlled using any one of a number of known techniques such as, for example, blocking the membrane with protein-containing solutions, addition of heterologous RNA, DNA, and SDS to the hybridization buffer, and treatment with RNase. Wash conditions are typically performed at or below stringency.

Without being bound thereby, it is believed that BVR can induce changes in the expression levels of regulatory cell cycle and cell signaling proteins in one or more of several ways. First, because BVR has been shown to be a kinase, BVR can regulate the activity of certain cell signaling molecules and, therefore, may indirectly modify expression levels of other cell cycle and cell signaling proteins. In this manner, it is believed that BVR overexpression can indirectly increase or decrease expression of such genes, whereas BVR inhibition can indirectly decrease or increase expression of other such genes. Second, BVR has been shown to regulate expression levels of proteins whose genes possess an AP-1 binding site in the upstream regulatory control regions, such as HO-1 and ATF-2. In this manner, BVR overexpression can directly increase expression of such genes whereas BVR inhibition can decrease expression of such genes.

As used herein, BVR variants and fragments can be substituted for BVR either in whole or in part.

Fragments of BVR preferably contain the leucine-zipper motif as listed above and any suitable nuclear localization signal, including the nuclear localization signal described above. Suitable fragments are capable of binding to the AP-1 binding site(s) in the promoter region of genes whose expression are to be modified, such as HO-1. Suitable fragments can be produced by several means.

Subclones of a gene encoding a known BVR can be produced using conventional molecular genetic manipulation for subcloning gene fragments, such as described by Sambrook et al., Molecular Cloning: A Laboratory Manual, Cold Springs Laboratory, Cold Springs Harbor, N.Y. (1989), and Ausubel et al. (ed.), Current Protocols in Molecular Biology, John Wiley & Sons (New York, N.Y.) (1999 and preceding editions), each of which is hereby incorporated by reference in its entirety. The subclones then are expressed in vitro or in vivo in bacterial cells to yield a smaller protein or polypeptide that can be tested for a particular activity.

In another approach, based on knowledge of the primary structure of the protein, fragments of a BVR gene may be synthesized using the PCR technique together with specific sets of primers chosen to represent particular portions of the protein. Erlich et al., Science 252:1643-51 (1991), which is hereby incorporated by reference in its entirety. These can then be cloned into an appropriate vector for expression of a truncated protein or polypeptide from bacterial cells as described above. For example, oligomers of at least about 15 to 20 nt in length can be selected from the nucleic acid molecule of SEQ ID NO: 2 for use as primers.

In addition, chemical synthesis can also be employed using techniques well known in the chemistry of proteins such as solid phase synthesis (Merrifield, J. Am. Chem. Assoc. 85:2149-2154 (1964), which is hereby incorporated by reference in its entirety) or synthesis in homogenous solution (Houbenweyl, Methods of Organic Chemistry, ed. E. Wansch, Vol. 15, I and II, Thieme, Stuttgart (1987), which is hereby incorporated by reference in its entirety).

Exemplary fragments include N-terminal, internal, and C-terminal fragments that possess a functional leucine zipper motif alone or in combination with other motifs, such as a nuclear localization signal.

Variants of suitable BVR proteins or polypeptides can also be expressed. Variants may be made by, for example, the deletion, addition, or alteration of amino acids that have either (i) minimal influence on certain properties, secondary structure, and hydropathic nature of the polypeptide or (ii) substantial effect on one or more properties of BVR. Variants of BVR can also be fragments of BVR that include one or more deletion, addition, or alteration of amino acids of the type described above. The BVR variant preferably contains a deletion, addition, or alteration of amino acids within one of the above-listed functional domains. The substituted or additional amino acids can be either L-amino acids, D-amino acids, or modified amino acids, preferably L-amino acids. Whether a substitution, addition, or deletion results in modification of BVR variant activity may depend, at least in part, on whether the altered amino acid is conserved. Conserved amino acids can be grouped either by molecular weight or charge and/or polarity of R groups, acidity, basicity, and presence of phenyl groups, as is known in the art.

Exemplary variants include the protein or polypeptides of SEQ ID Nos: 1 and 3, which have single or multiple amino acid residue substitutions, including, without limitation, SEQ ID NO: 1 as modified by one or more of the following variations: (i) Gly¹⁷-to-Ala within the nucleotide binding domain, (ii) Ser⁴⁴-to-Ala within one of the kinase motifs, (iii) Cys⁷⁴-to-Ala within a substrate binding domain, (iv) Lys⁹²His⁹³-to-Ala-Ala within the oxidoreductase motif, (v) G²²²LKRNR²²⁷-to-VIGSTG within the nuclear localization signal, and (vi) Cys²⁸¹-to-Ala within the zinc finger domain, and Lys²⁹⁶-to-Ala at the C terminus within a substrate binding domain (i.e., protein kinase inhibitory domain).

Variants may also include, for example, a polypeptide conjugated to a signal (or leader) sequence at the N-terminal end of the protein which co-translationally or post-translationally directs transfer of the protein. The polypeptide may also be conjugated to a linker or other sequence for ease of synthesis, purification, identification, or therapeutic use (i.e., delivery) of the polypeptide.

Another variant type of BVR is a fusion polypeptide that includes a fragment of BVR containing the functional leucine zipper motif (but not the endogenous nuclear localization signal) and a functional nuclear localization signal. The fusion protein can be expressed or synthesized using an in-frame gene fusion according to known techniques in the art. A number of nuclear localization signals have been identified in the art and can be utilized in combination with the fragment of BVR to obtain the fusion protein, which is targeted for uptake into the cell nucleus following its introduction into the cell whose cell cycle or cell signaling pathways are to be modified in accordance with the present invention. Production of chimeric genes encoding such fusion proteins can be carried out as described infra.

The BVR protein or polypeptide (or fragment or variant thereof) can be recombinantly produced, isolated, and then purified, if necessary. When recombinantly produced, the biliverdin reductase protein or polypeptide (or fragment or variant thereof) is expressed in a recombinant host cell, typically, although not exclusively, a prokaryote.

When a prokaryotic host cell is selected for subsequent transformation, the promoter region used to construct the recombinant DNA molecule (i.e., transgene) should be appropriate for the particular host. The DNA sequences of eukaryotic promoters, as described infra for expression in eukaryotic host cells, differ from those of prokaryotic promoters. Eukaryotic promoters and accompanying genetic signals may not be recognized in or may not function in a prokaryotic system, and, further, prokaryotic promoters are not recognized and do not function in eukaryotic cells.

Similarly, translation of mRNA in prokaryotes depends upon the presence of the proper prokaryotic signals which differ from those of eukaryotes. Efficient translation of mRNA in prokaryotes requires a ribosome binding site called the Shine-Dalgarno (“SD”) sequence on the mRNA. This sequence is a short nucleotide sequence of mRNA that is located before the start codon, usually AUG, which encodes the amino-terminal methionine of the protein. The SD sequences are complementary to the 3′-end of the 16S rRNA (ribosomal RNA) and probably promote binding of mRNA to ribosomes by duplexing with the rRNA to allow correct positioning of the ribosome. For a review on maximizing gene expression, see Roberts and Lauer, Methods in Enzymology, 68:473 (1979), which is hereby incorporated by reference in its entirety.

Promoters vary in their “strength” (i.e., their ability to promote transcription). For the purposes of expressing a cloned gene, it is desirable to use strong promoters in order to obtain a high level of transcription and, hence, expression of the gene. Depending upon the host cell system utilized, any one of a number of suitable promoters may be used. For instance, when cloning in E. coli, its bacteriophages, or plasmids, promoters such as the T7 phage promoter, lac promoter, trp promoter, recA promoter, ribosomal RNA promoter, the P_{R} and P_{L} promoters of coliphage lambda and others, including but not limited, to lacUV5, oinpF, bla, lpp, and the like, may be used to direct high levels of transcription of adjacent DNA segments. Additionally, a hybrid trp-lacUV5 (tac) promoter or other E. coli promoters produced by recombinant DNA or other synthetic DNA techniques may be used to provide for transcription of the inserted gene.

Bacterial host cell strains and expression vectors may be chosen which inhibit the action of the promoter unless specifically induced. In certain operons, the addition of specific inducers is necessary for efficient transcription of the inserted DNA. For example, the lac operon is induced by the addition of lactose or IPTG (isopropylthio-beta-D-galactoside). A variety of other operons, such as trp, pro, etc., are under different controls.

Specific initiation signals are also required for efficient gene transcription and translation in prokaryotic cells. These transcription and translation initiation signals may vary in “strength” as measured by the quantity of gene specific messenger RNA and protein synthesized, respectively. The DNA expression vector, which contains a promoter, may also contain any combination of various “strong” transcription and/or translation initiation signals. For instance, efficient translation in E. coli requires a Shine-Dalgarno (“SD”) sequence about 7-9 bases 5′ to the initiation codon (“ATG”) to provide a ribosome binding site. Thus, any SD-ATG combination that can be utilized by host cell ribosomes may be employed. Such combinations include, but are not limited to, the SD-ATG combination from the cro gene or the N gene of coliphage lambda, or from the E. coli tryptophan E, D, C, B or A genes. Additionally, any SD-ATG combination produced by recombinant DNA or other techniques involving incorporation of synthetic nucleotides may be used.

Mammalian cells can also be used to recombinantly produce BVR or fragments or variants thereof. Mammalian cells suitable for carrying out the present invention include, among others: COS (e.g., ATCC No. CRL 1650 or 1651), BHK (e.g., ATCC No. CRL 6281), CHO (ATCC No. CCL 61), HeLa (e.g., ATCC No. CCL 2), 293 (ATCC No. 1573), CHOP, and NS-1 cells.

Suitable expression vectors for directing expression in mammalian cells generally include a promoter, as well as other transcription and translation control sequences known in the art. Common promoters include SV40, MMTV, metallothionein-1, adenovirus E1a, CMV, immediate early, immunoglobulin heavy chain promoter and enhancer, and RSV-LTR.

Regardless of the selection of host cell, once the DNA molecule coding for a biliverdin reductase protein or polypeptide, or fragment or variant thereof, has been ligated to its appropriate regulatory regions (or chimeric portions) using well known molecular cloning techniques, it can then be introduced into a suitable vector or otherwise introduced directly into a host cell using transformation protocols well known in the art (Sambrook et al., Molecular Cloning: A Laboratory Manual, Second Edition, Cold Spring Harbor Press, NY (1989), which is hereby incorporated by reference in its entirety).

When an expression vector is used for purposes of in vivo transformation to induce or inhibit of BVR expression in a target cell, promoters of varying strength can be employed depending on the degree of enhancement of suppression desired. One of skill in the art can readily select appropriate mammalian promoters based on their strength as a promoter. Alternatively, an inducible promoter can be employed for purposes of controlling when expression or suppression of BVR is desired. One of skill in the art can readily select appropriate inducible mammalian promoters from those known in the art. Finally, tissue specific mammalian promoters can be selected to restrict the efficacy of any gene transformation system to a particular tissue. Tissue specific promoters are known in the art and can be selected based upon the tissue or cell type to be treated.

The recombinant molecule can be introduced into host cells via transformation, particularly transduction, conjugation, mobilization, or electroporation. Suitable host cells include, but are not limited to, bacteria, virus, yeast, mammalian cells, insect, plant, and the like. The host cells, when grown in an appropriate medium, are capable of expressing the biliverdin reductase, or fragment or variant thereof, which can then be isolated therefrom and, if necessary, purified. The biliverdin reductase, or fragment or variant thereof, is preferably produced in purified form (preferably at least about 60%, more preferably 80%, pure) by conventional techniques.

A further aspect of the present invention relates to an antisense nucleic acid molecule capable of hybridizing with an RNA transcript coding for BVR. Basically, the antisense nucleic acid is expressed from a transgene which is prepared by ligation of a DNA molecule, coding for BVR, or a fragment or variant thereof, into an expression vector in reverse orientation with respect to its promoter and 3′ regulatory sequences. Upon transcription of the DNA molecule, the resulting RNA molecule will be complementary to the “iRNA transcript coding for the actual protein or polypeptide product. Ligation of DNA molecules in reverse orientation can be performed according to known techniques which are standard in the art.

Such antisense nucleic acid molecules of the invention may be used in gene therapy to treat or prevent various disorders. For a discussion of the regulation of gene expression using anti-sense genes, see Weintraub et al., Reviews-Trends in Genetics, 1(1) (1986), which is hereby incorporated by reference in its entirety. As discussed infra, recombinant molecules including an antisense sequence or oligonucleotide fragment thereof, may be directly introduced into cells of tissues in vivo using delivery vehicles such as retroviral vectors, adenoviral vectors and DNA virus vectors. They may also be introduced into cells in vivo using physical techniques such as microinjection and electroporation or chemical methods such as coprecipitation and incorporation of DNA into liposomes.

As an alternative to antisense BVR mRNA, siRNA can be used to decrease the cellular or nuclear concentration of BVR. Preferably, an siRNA is less than about 30 nucleotides in length. Specific siRNAs suitable for downregulating expression levels/activity of cellular BVR can be identified at the Ambion, Inc. Internet site, which provides a target sequence to siRNA converter. By introducing the cDNA sequence of BVR, the Ambion, Inc. Internet site will identify sense and anti-sense strands of the siRNA molecule, as well as identify the DNA construct needed to express the siRNA. An exemplary siRNA sequences (in the form of a duplex) are as follows:

(SEQ ID NO:8)

5′-UCCUCAGCGUUCCUGAACCUG;

(SEQ ID NO:9)

3′-AGGAGUCGCAAGGACUUGGAC.

As noted above, the present application allows for the upregulation or downregulation of cell cycle proteins and cell signaling proteins by modifying the BVR levels in cells.

Exemplary cell signaling proteins that can be upregulated by BVR or fragments or variants thereof include, without limitation, creb-2, bfl-1, IAP-1, IAP-2, p16Ink4, beta-casein, p450XIX, GADD45, HIP and RPL13. These and other proteins are shown in Examples 4 and 5 infra.

Exemplary cell signaling proteins that can be downregulated by BVR or fragments or variants thereof include, without limitation, p27Kip1, p15Ink2b, p18 (cdk4 inhibitor), CDX1, FASN and Stra6. These and other proteins are shown in Examples 4 and 5 infra.

Exemplary cell cycling proteins that can be upregulated by BVR or fragments or variants thereof include, without limitation, cyclins A, E1 and E2, CDK15a, CDC7, cdk1, cdk2, cdk8, Cks2, Cks1p9, Cul1, Cul2, Cul3, E2F-3, MAD2L1, MCM6, Rbx1, and beta-actin. These and other proteins are also shown in Examples 4 and 5 infra.

Exemplary cell cycling proteins that can be downregulated by BVR or fragments or variants thereof include, without limitation, RAD50, cdk4, CDK10, and RPL13A. These and other proteins are also shown in Examples 4 and 5 infra.

By virtue of BVR-induced up- or down-regulation of the above-listed cell signaling proteins and cell cycling proteins, it is believed that regulation of BVR levels in cells can thereby modify cell signaling and/or cell cycling events under their control. As a result, it is believed that cellular BVR levels can treat or prevent disease conditions or disorders that involve one or more of the above-listed proteins. Such disease conditions or disorders are described in the subsequent sections that address the functions and pathways in which the proteins are involved.

### Bax

Development as well as maintenance of many adult tissues is achieved by several dynamically regulated processes that include cell proliferation, differentiation, and programmed cell death. Oltvai et al., Cell 74:609-619 (1993), which is hereby incorporated by reference in its entirety, noted that, in the latter process, cells are eliminated by a highly characteristic suicide program called apoptosis. The best-defined genetic pathway of cell death exists in the nematode Caenorhabditis elegans. Two autosomal recessive death effector genes, ced-3 and ced-4, are required for the death of all 131 cells destined to die during worm development. One autosomal dominant death repressor gene, ced-9, can save those cells in its gain-of-function form. This implies that both effector and repressor genes also exist within each mammalian cell death pathway. BCL2 is one such mammalian gene that has been identified; it functions as a repressor of programmed cell death.

Oltvai et al., Cell 74:609-619 (1993), which is hereby incorporated by reference in its entirety, showed that BCL2 associates in vivo with a 21-kD program partner, Bax. Bax shows extensive amino acid homology with BCL2 and forms homodimers and heterodimers with BCL2 in vivo. When Bax predominates, programmed cell death is accelerated, and the death repressor activity of BCL2 is countered. These findings suggest a model in which the ratio of BCL2 to Bax determines survival or death following an apoptotic stimulus.

The Bax gene promoter region contains 4 motifs with homology to consensus p53-binding sites. In cotransfection assays using p53-deficient tumor cell lines, Miyashita and Reed, Cell 80:293-299 (1995), which is hereby incorporated by reference in its entirety, found that wildtype but not mutant p53 expression plasmids transactivated a reporter gene plasmid that utilized the Bax gene promoter to drive transcription of chloramphenicol acetyltransferase. Introduction of mutations into the consensus p53-binding site sequences abolished p53 responsiveness of the reporter gene plasmids. Taken together, the results suggested that Bax is a primary-response gene for p53 and is involved in a p53-regulated pathway for induction of apoptosis.

Apte et al., Genomics 26:592-594 (1995), which is hereby incorporated by reference in its entirety, isolated a Bax cDNA clone in which the mRNA encoded by exon 3 was absent. The skipping of exon 3 predicted the existence of an interstitially truncated form of the major Bax protein (Bax-alpha), termed Bax-delta. Unlike two previously described variant forms, Bax-delta retains the functionally critical C-terminal membrane anchor region, as well as the BCL2 homology 1 and 2 (BH1 and BH2) domains.

Carton et al., Hum. Molec. Genet. 11:675-687 (2002), which is hereby incorporated by reference in its entirety, examined the expression of Bax in 55 patients with glioblastoma multiforme, the most common and aggressive form of brain tumors. The authors identified a novel form of Bax, designated Bax-psi, which was present in 24% of the patients. Bax-psi is an N-terminal truncated form of Bax which results from a partial deletion of exon 1 of the Bax gene. Bax-psi and the wildtype form, Bax-alpha, are encoded by distinct mRNAs, both of which are present in normal tissues. Glial tumors expressed either Bax-alpha or Bax-psi proteins, an apparent consequence of an exclusive transcription of the corresponding mRNAs. The Bax-psi protein was preferentially localized to mitochondria and was a more powerful inducer of apoptosis than Bax-alpha. Bax-psi tumors exhibited slower proliferation in Swiss nude mice, and this feature could be circumvented by the coexpression of the BCL2 transgene, the functional antagonist of Bax. The expression of Bax-psi correlated with a longer survival in patients (18 months versus 10 months for Bax-alpha patients). The authors hypothesized a beneficial involvement of the psi variant of Bax in tumor progression.

During transduction of an apoptotic signal into the cell, there is an alteration in the permeability of the membranes of the cell's mitochondria, which causes the translocation of the apoptogenic protein cytochrome c into the cytoplasm, which in turn activates death-driving proteolytic proteins known as caspases. The BCL2 family of proteins, whose members may be antiapoptotic or proapoptotic, regulates cell death by controlling this mitochondrial membrane permeability during apoptosis. Shimizu et al., Nature 399:483-487 (1999), which is hereby incorporated by reference in its entirety, created liposomes that carried the mitochondrial porin channel VDAC to show that the recombinant proapoptotic proteins Bax and Bak accelerate the opening of VDAC, whereas the antiapoptotic protein BCLXL closes VDAC by binding to it directly. Bax and Bak allow cytochrome c to pass through VDAC out of liposomes, but passage is prevented by BCLXL. In agreement with this, VDAC1-deficient mitochondria from a mutant yeast did not exhibit a Bax/Bak-induced loss in membrane potential and cytochrome c release, both of which were inhibited by BCLXL. Shimizu et al., Nature 399:483-487 (1999), which is hereby incorporated by reference in its entirety, concluded that the BCL2 family of proteins bind to the VDAC in order to regulate the mitochondrial membrane potential and the release of cytochrome c during apoptosis.

To assess the role of Bax in drug-induced apoptosis in human colorectal cancer cells (HCT116 cells), Zhang et al., Science 290:989-992 (2000), which is hereby incorporated by reference in its entirety, generated cells that lacked functional Bax genes. Such cells were partially resistant to the apoptotic effects of the chemotherapeutic agent 5-fluorouracil, but apoptosis was not abolished. In contrast, the absence of Bax completely abolished the apoptotic response to the chemopreventive agent sulindac and other nonsteroidal antiinflammatory drugs (NSAIDs). NSAIDs inhibited the expression of the antiapoptotic protein BCLXL, resulting in an altered ratio of Bax to BCLXL and subsequent mitochondria-mediated cell death. Zhang et al., Science 290:989-992 (2000), which is hereby incorporated by reference in its entirety, concluded that their results establish an unambiguous role for Bax in apoptotic processes in human epithelial cancers and may have implications for cancer chemoprevention strategies.

Studies of Bax-deficient mice indicated that the pro-apoptotic Bax molecule can function as a tumor suppressor. For that reason, Meijerink et al., Blood 91:2991-2997 (1998), which is hereby incorporated by reference in its entirety, examined human hematopoietic malignancies and found that approximately 21% of lines possessed mutations in Bax, perhaps most commonly in the acute lymphoblastic leukemia subset. Approximately half were nucleotide insertions or deletions within a deoxyguanosine (G8) tract, resulting in a proximal frameshift and loss of immunodetectable Bax protein. Other Bax mutants bore single amino acid substitutions within BH1 or BH3 domains, demonstrated altered patterns of protein dimerization, and had lost death-promoting activity.

The proapoptotic Bax protein induces cell death by acting on the mitochondria. Bax binds to the permeability transition pore complex (PTPC), a composite proteaceous channel that is involved in the regulation of mitochondrial membrane permeability. Marzo et al., Science 281:2027-2031 (1998), which is hereby incorporated by reference in its entirety, found that immunodepletion of Bax from PTPC or purification of PTPC from Bax-deficient mice yielded a PTPC that could not permeabilize membranes in response to atractyloside, a proapoptotic ligand of the adenine nucleotide translocator (ANT). Bax and ANT communoprecipitated and interacted in the yeast 2-hybrid system. Ectopic expression of Bax induced cell death in wildtype but not in ANT-deficient yeast. Recombinant Bax and purified ANT, but neither of them alone, efficiently formed atractyloside-responsive channels in artificial membranes. Hence, the proapoptotic molecule Bax and the constitutive mitochondrial protein ANT cooperate within the PTPC to increase mitochondrial membrane permeability and to trigger cell death.

The caspase-activated form of BID, tBID, triggers the homooligomerization of multidomain conserved proapoptotic family members BAK or Bax, resulting in the release of cytochrome c from mitochondria. Wei et al., Science 292:727-730 (2001), which is hereby incorporated by reference in its entirety, found that cells lacking both BAK and Bax, but not cells lacking only one of these components, are completely resistant to tBID-induced cytochrome c release and apoptosis. Moreover, doubly deficient cells are resistant to multiple apoptotic stimuli that act through disruption of mitochondrial function: staurosporine, ultraviolet radiation, growth factor deprivation, etoposide, and the endoplasmic reticulum stress stimuli thapsigargin and tunicamycin. Thus, Wei et al., Science 292:727-730 (2001), which is hereby incorporated by reference in its entirety, concluded that activation of a ‘multidomain’ proapoptotic member, BAK or Bax, appears to be an essential gateway to mitochondrial dysfunction required for cell death in response to diverse stimuli.

Polycyclic aromatic hydrocarbons (PAHs) are toxic chemicals released into the environment by fossil fuel combustion. Oocyte destruction and ovarian failure occur in PAH-treated mice, and cigarette smoking causes early menopause in women. In many cells, PAHs activate the aromatic hydrocarbon receptor (AHR), a member of the Per-Arnt-Sim family of transcription factors. The AHR is also activated by dioxin, one of the most intensively studied environmental contaminants. Matikainen Nature Genet. 28:355-360 (2001), which is hereby incorporated by reference in its entirety, demonstrated that exposure of mice to PAHs induces the expression of Bax in oocytes, followed by apoptosis. Ovarian damage caused by PAHs is prevented by Alir or Bax inactivation. Oocytes microinjected with a Bax promoter-reporter construct show Ahr-dependent transcriptional activation after PAH, but not dioxin, treatment, consistent with findings that dioxin is not cytotoxic to oocytes. This difference in the action of PAHs versus dioxin is conveyed by a single basepair flanking each Ahr response element in the Bax promoter. Oocytes in human ovarian biopsies grafted into immunodeficient mice also accumulated Bax and underwent apoptosis after PAH exposure in vivo. Thus, AHR-driven Bax transcription is a novel and evolutionarily conserved cell-death signaling pathway responsible for environmental toxicant-induced ovarian failure.

To investigate the relationship between apoptosis and the BCL2/Bax system in the human corpus luteum, Sugino et al., J. Clin. Endocr. Metab. 85:4379-4386 (2000), which is hereby incorporated by reference in its entirety, examined the frequency of apoptosis and expression of BCL2 and Bax in the corpus luteum during the menstrual cycle and in early pregnancy. Immunohistochemistry revealed BCL2 expression in the luteal cells in the midluteal phase and early pregnancy, but not in the regressing corpus luteum. In contrast, Bax immunostaining was observed in the regressing corpus luteum, but not in the midluteal phase or early pregnancy. The BCL2 mRNA levels in the corpus luteum during the menstrual cycle were highest in the midluteal phase and lowest in the regressing corpus luteum. In the corpus luteum of early pregnancy, BCL2 mRNA levels were significantly higher than those in the midluteal phase. In contrast, Bax mRNA levels were highest in the regressing corpus luteum and remarkably low in the corpus luteum of early pregnancy. When corpora lutea of the midluteal phase were incubated with CG, CG significantly increased the mRNA and protein levels of BCL2 and significantly decreased those of Bax. Sugino et al., J. Clin. Endocr. Metab. 85:4379-4386 (2000), which is hereby incorporated by reference in its entirety, concluded that BCL2 and Bax may play important roles in the regulation of the life span of the human corpus luteum by controlling the rate of apoptosis. CG may act to prolong the life span of the corpus luteum by increasing BCL2 expression and decreasing Bax expression when pregnancy occurs.

LeBlanc et al., Nature Med. 8:274-281 (2002), which is hereby incorporated by reference in its entirety, demonstrated that Bax can be essential for death receptor-mediated apoptosis in cancer cells. Bax-deficient human colon carcinoma cells were resistant to death-receptor ligands, whereas Bax-expressing sister clones were sensitive. Bax was dispensable for apical death-receptor signaling events including caspase-8 activation, but crucial for mitochondrial changes and downstream caspase activation. Treatment of colon cancer cells deficient in DNA mismatch repair with the TRAIL selected in vitro or in vivo for refractory subclones with Bax frameshift mutations including deletions at a novel site. Chemotherapeutic agents upregulated expression of the TRAIL receptor DR5 and the Bax homolog BAK in Bax −/− cells, and restored TRAIL sensitivity in vitro and in vivo. Thus, Bax mutation in mismatch repair-deficient tumors can cause resistance to death receptor-targeted therapy, but pre-exposure to chemotherapy rescues tumor sensitivity.

Guo et al., Nature 423:456-461 (2003), which is hereby incorporated by reference in its entirety, found that Bax coimmunoprecipitated with humanin, a peptide with neuroprotective activities against Alzheimer disease-associated insults, and that humanin rescued rat hippocampal neurons from Bax-induced lethality. Humanin prevented the translocation of Bax from the cytosol to the mitochondria and suppressed cytochrome c release. Guo et al., Nature 423:456-461 (2003), which is hereby incorporated by reference in its entirety, noted that the predicted humanin peptides from the nuclear-encoded peptide and the mitochondrial-encoded peptide were both able to bind Bax and prevent apoptosis. The authors suggested that the HN gene arose from mitochondria and transferred to the nuclear genome, providing a protective mechanism for additional organelles.

Cancers of the microsatellite mutator phenotype (MMP) show exaggerated genomic instability at simple repeat sequences. The human Bax gene contains a tract of 8 consecutive deoxyguanosines in the third coding exon, spanning codons 38 to 41. To determine whether this sequence is a mutational target in MMP(+) tumor cells, Rampino et al., Science 275: 967-969 (1997), which is hereby incorporated by reference in its entirety, amplified by PCR the region containing the (G)8 tract from various MMP(+) tumor cell lines. This analysis revealed band shifts, suggestive of 1-bp insertions and deletions in some of these tumor cells. Homozygous (or hemizygous) frameshift insertion or deletion mutations in Bax were found in multiple primary colorectal cancers as well as colorectal cancer cell lines. The resulting frameshift was thought to interfere with the suppressor role of the wildtype Bax gene. Rampino et al., Science 275: 967-969 (1997), which is hereby incorporated by reference in its entirety, noted that colon tumors of the MMP type typically do not contain p53 mutations, in contrast with those of the suppressor pathway. Once the MMP is manifested (after the occurrence of mutator mutations in, for example, mismatch repair genes), mutations at the Bax (G)8 hotspot would be more likely to occur than other frameshift or missense mutations in p53. In tumor cells with frameshift Bax mutations, transcriptional activation of Bax by wildtype p53 would be irrelevant. In cancer of the MMP, the generation of thousands of DNA mismatches during every replication of each MMP(+) tumor cell may trigger the p53-mediated apoptotic response to DNA damage. But the response would be futile because the chain leading to apoptosis is broken in a downstream link. Therefore, Rampino et al., Science 275: 967-969 (1997), which is hereby incorporated by reference in its entirety, speculated that Bax mutations eliminate the selective pressure for p53 mutations during colorectal tumorigenesis.

Female mammals are endowed with a finite number of oocytes at birth, each enclosed by a single layer of somatic (granulosa) cells in a primordial follicle. The fate of most follicles is atretic degeneration, a process that culminates in near exhaustion of the oocyte reserve at approximately the fifth decade of life in women, leading to menopause. Apoptosis has a fundamental role in follicular atresia, and several studies had indicated that Bax, which is expressed in both granulosa cells and oocytes, may be central to ovarian cell death. Perez et al., Nature Genet. 21:200-203 (1999), which is hereby incorporated by reference in its entirety, showed that young adult female mice homozygous for disruption of the Bax gene, (Bax −/−), possessed 3-fold more primordial follicles in their ovarian reserve than their wildtype sisters, and that this surfeit of follicles was maintained in advanced chronologic age, such that 20- to 22-month-old female Bax −/− mice possessed hundreds of follicles at all developmental stages and exhibited ovarian steroid-driven uterine hypertrophy. These observations contrasted with the ovarian and uterine atrophy seen in aged wildtype female mice. Aged female Bax −/− mice failed to become pregnant when housed with young adult males; however, metaphase II oocytes could be retrieved from, and corpora lutea formed in, ovaries of aged Bax −/− females following superovulation with exogenous gonadotropins, and some oocytes were competent for in vitro fertilization and early embryogenesis. Therefore, ovarian lifespan could be extended by selectively disrupting Bax function, but other aspects of normal reproductive performance remained defective in aged Bax −/− female mice.

The central nervous system (CNS) of Atm null mice shows a pronounced defect in apoptosis induced by genotoxic stress, suggesting that ATM functions to eliminate neurons with excessive genomic damage. Chong et al., Proc. Nat. Acad. Sci. 97: 889-894 (2000), which is hereby incorporated by reference in its entirety, reported that the death effector Bax is required for a large proportion of Atin-dependent apoptosis in the developing CNS after ionizing radiation (IR). Although many of the same regions of the CNS in both Bax −/− and Atm −/− mice were radioresistant, mice nullizygous for both Bax and Atm showed additional reduction in IR-induced apoptosis in the CNS. Therefore, although the major IR-induced apoptotic pathway in the CNS requires Atm and Bax, a p53-dependent collateral pathway exists that has both Atm- and Bax-independent branches. Furthermore, Atm-and Bax-dependent apoptosis in the CNS also required caspase-3 activation. These data implicated Bax and caspase-3 as death effectors in neurodegenerative pathways.

Proapoptotic Bcl2 family members have been proposed to play a central role in regulating apoptosis, yet mice lacking Bax display limited phenotypic abnormalities. Lindsten et al., Molec. Cell 6:1389-1399 (2000), which is hereby incorporated by reference in its entirety, found that Bak −/− mice were developmentally normal and reproductively fit and failed to develop any age-related disorders. However, when Bak-deficient mice were mated to Bax-deficient mice to create mice lacking both genes, the majority of Bax−/− Bak−/− animals died perinatally, with fewer than 10% surviving into adulthood. Bax−/− Bak−/− mice displayed multiple developmental defects, including persistence of interdigital webs, an imperforate vaginal canal, and accumulation of excess cells within both the central nervous and hematopoietic systems. Thus, the authors concluded that Bax and Bak have overlapping roles in the regulation of apoptosis during mammalian development and tissue homeostasis.

Scorrano et al., Science 300:135-139 (2003), which is hereby incorporated by reference in its entirety, found that mouse embryonic fibroblasts deficient for Bax and Bak had a reduced resting concentration of calcium in the endoplasmic reticulum (ER) that resulted in decreased uptake of calcium by mitochondria after calcium release from the ER. Expression of SERCA (sarcoplasmic-endoplasmic reticulum calcium adenosine triphosphatase) corrected ER calcium concentration and mitochondrial calcium uptake in double knockout cells, restoring apoptotic death in response to agents that release calcium from intracellular stores, such as arachidonic acid, C2-ceramide, and oxidative stress. In contrast, targeting of Bax to mitochondria selectively restored apoptosis to ‘BH3-only’ signals. A third set of stimuli, including many intrinsic signals, required both ER-released calcium and the presence of mitochondrial Bax or Bak to fully restore apoptosis. Scorrano et al. (2003), which is hereby incorporated by reference in its entirety, concluded that Bax and BAK operate in both the ER and the mitochondria as an essential gateway for selected apoptotic signals.

Garcia-Barros et al., Science 300:1155-1159 (2003), which is hereby incorporated by reference in its entirety, investigated the hypothesis that tumor response to radiation is determined not only by tumor cell type but also by microvascular sensitivity. MCA/129 fibrosarcomas and B16F1 melanomas grown in apoptosis-resistant ‘acid sphingomyelinase’ (asmase)-deficient or Bax-deficient mice displayed markedly reduced baseline microvascular endothelial apoptosis and grew 200 to 400% faster than tumors on wildtype microvasculature. Thus, Garcia-Barros et al., Science 300:1155-1159 (2003), which is hereby incorporated by reference in its entirety, concluded that endothelial apoptosis is a homeostatic factor regulating angiogenesis-dependent tumor growth. Moreover, these tumors exhibited reduced endothelial apoptosis upon irradiation and, unlike tumors in wildtype mice, they were resistant to single-dose radiation up to 20 Gy. Garcia-Barros et al., Science 300:1155-1159 (2003), which is hereby incorporated by reference in its entirety, concluded that microvascular damage regulates tumor cell response to radiation at the clinically relevant dose range.

Rampino et al., Science 275:967-969 (1997), which is hereby incorporated by reference in its entirety, found that more than 50% (21 of 41) of human MMP(+) colon adenocarcinomas they examined had frameshift mutations in a tract of 8 deoxyguanosines within the Bax gene in the third coding exon, spanning codons 38 to 41. These mutations were absent in MM(−) tumors and were significantly less frequent in G8 tracts from other genes. Frameshift mutations were present in both Bax alleles and some MMP(+) colon tumor cell lines and in primary tumors. These results suggested that inactivating Bax mutations are selected for during the progression of colorectal MMP(+) tumors and that the wildtype Bax gene plays a suppressor role in a p53-independent pathway for colorectal carcinogenesis.

In a patient with T-cell acute lymphoblastic leukemia, Meijerink et al., Blood 91:2991-2997 (1998), which is hereby incorporated by reference in its entirety, found a gly67-to-arg missense mutation of the Bax gene.

In several cell lines from patients with T-cell acute lymphoblastic leukemia, Meijerink et al., Blood 91:2991-2997 (1998), which is hereby incorporated by reference in its entirety, found deletion of 7 guanine residues from a simple tract of 8 such residues encompassing codons 38 to 41 of the Bax gene.

### BFL-1

Programmed cell death (apoptosis) plays an important role in embryonic development, deletion of autoreactive T lymphocytes, and homeostasis. Genes regulating apoptosis include p53, a tumor suppressor gene, MYC, a protooncogene, and BCL2. Lin et al., J. Immun. 151:1979-1988 (1993), which is hereby incorporated by reference in its entirety, isolated a novel mouse cDNA sequence, designated BCL2-related protein A1 (Bfl-1) by them, and identified it as a member of the BCL2 family of apoptosis regulators by the predicted protein sequence. Lin et al., Blood 87: 983-992 (1996), which is hereby incorporated by reference in its entirety, demonstrated that the Al protein, although regulated differently from BCL2, has similar antiapoptotic activity.

Choi et al., Oncogene 11:1693-1698 (1995), which is hereby incorporated by reference in its entirety, isolated a BCL2-related gene from human fetal liver. Homology to the BH1 and BH2 domains of BCL2 was striking. Bfl-1 is abundantly expressed in bone marrow and at a low level in some other tissues. A correlation was noted between the expression level of Bfl-1 and the development of stomach cancer in 8 sets of clinical samples. Choi et al., Oncogene 11:1693-1698 (1995), which is hereby incorporated by reference in its entirety, speculated that Bfl-1 is involved in the promotion of cell survival during development or progression of stomach cancer. Choi et al., Mammalian Genome 8: 781-782 (1997), which is hereby incorporated by reference in its entirety, showed that Bfl-1 is the human homolog of murine A1.

D'Sa-Eipper et al., Cancer Res. 56:3879-3882 (1996), which is hereby incorporated by reference in its entirety, showed that the Bfl-1 protein suppresses apoptosis induced by the p53 tumor suppressor protein in a manner similar to other BCL2 family members. The Bfl-1 gene showed a dominant cooperating oncogenic activity with the EIA oncogene in transformation of primary rodent epithelial cells.

Using mast cells from wildtype and Bfl-1-deficient mice, Xiang et al., J. Exp. Med. 194:1561-1569 (2001), which is hereby incorporated by reference in its entirety, showed that knockout mice had normal numbers of mast cells in skin, lung, and spleen. Bone marrow-derived mast cells from normal mice expressed Bfl-1 after activation and, like Bfl-1-deficient mice, released granule mediators. However, mast cells from Bfl-1-deficient mice did not survive allergen activation in vitro, and mast cell number was reduced in vivo after allergen sensitization and provocation. Xiang et al., J. Exp. Med. 194:1561-1569 (2001), which is hereby incorporated by reference in its entirety, proposed that Bfl-1 could be a target in the treatment of allergic diseases.

### Beta-Casein

The caseins have been shown to be members of a multigene family in at least 2 species, cow and man. They are among the most rapidly diverging groups of proteins. Bovine milk contains 4 caseins, 2 alpha, 1 beta, and 1 kappa. Human milk, on the other hand, contains only 2 caseins, beta and kappa. Beta-casein is the major casein in human milk, accounting for as much as 30% of its total protein mass. In addition to being the primary source of essential amino acids, beta-casein, in concert with kappa-casein, forms micelles that transport calcium and phosphorus to the developing infant. Menon and Ham, Nucleic Acids Res. 17:2869 (1989) and Lonnerdal et al., FEBS Lett. 269:153-156 (1990), which are hereby incorporated by reference in their entirety, cloned cDNAs for human beta-casein.

Comparison with other species indicates that the caseins are among the most rapidly evolving proteins. Nevertheless, a number of well-conserved residues are distributed along its entire length. These residues are thought to play an important role in conserving the 3-dimensional structure of the protein. Menon et al., Genomics 12:13-17 (1992), which is hereby incorporated by reference in its entirety, showed that in relation to the beta-casein of other species, the mature protein in the human shows a deletion of amino acids encoded by exon 3. They concluded that an interruption of the polypyrimidine tract adjacent to the 5-prime end of the exon 3 sequence may account for the omission of the exon from human beta-casein mRNA. They stated that a broader sampling would be required for a firm conclusion that exon 3 is never expressed in human beta-casein. Nevertheless, the lack of expression of exon 3 is at the very least a frequent occurrence in humans and may well be species-specific. Exon 3 encodes 9 residues, including 2 additional phosphorylation sites, serine residues 7 and 8. The N-terminal phosphoserine/phosphothreonine amino acids of beta-casein are important to the biologic function of the molecule, and variations in their number could affect the overall quality of milk.

### GADD45

Ionizing radiation can induce specific genes in mammalian and other eukaryotic cells. Two such genes, often referred to as GADD45 and GADD153, are strongly and coordinately induced by ultraviolet radiation and alkylating agents in human and hamster cells. (These genes are designated GADD for ‘growth arrest- and DNA damage-inducible.’) Papathanasiou et al., Molec. Cell. Biol. 11:1009-1016 (1991), which is hereby incorporated by reference in its entirety, found that GADD45 but not GADD153 is strongly induced by x-rays in human cells. No induction was seen after treatment with a known activator of protein kinase C. Therefore, GADD45 is the only known x-ray responsive gene whose induction is not mediated by PKC. Sequence analysis of human and hamster cDNA clones demonstrated that the gene has been highly conserved and encodes a novel 165-amino acid polypeptide that is 96% identical in the 2 species. In cell lines from 4 patients with ataxia-telangiectasia, Papathanasiou et al., Molec. Cell. Biol 11:1009-1016 (1991), which is hereby incorporated by reference in its entirety, demonstrated that induction by x-ray of GADD45 mRNA was reduced in comparison to the normal.

The stress-responsive p38 and JNK mitogen-activated protein kinase (MAPK) pathways regulate cell cycle and apoptosis. A human MAP3K, MTK1, mediates activation of both p38 and JNK in response to environmental stresses. By screening a placenta cDNA library using a yeast 2-hybrid method, Takekawa and Saito, Cell 95:521-530 (1998), which is hereby incorporated by reference in its entirety, isolated cDNAs encoding 3 related proteins, GADD45A, GADD45-beta, and GADD45-gamma (GADD45G), that bound to an N-terminal domain of MTK1. GADD45A, GADD45B, and GADD45G share 55 to 58% amino acid identity. These proteins activated MTK1 kinase activity, both in vivo and in vitro. All 3 GADD45-like genes were induced by environmental stresses, including methyl methanesulfonate, UV, and gamma irradiation. Expression of the GADD45-like genes induced p38/JNK activation and apoptosis, which could be partially suppressed by coexpression of a dominant inhibitory MTK1 mutant protein. Northern blot analysis detected moderate expression of a 1.4-kb GADD45A transcript in heart, skeletal muscle, and kidney, with little or no expression in brain, placenta, lung, liver, and pancreas. Takekawa and Saito, Cell 95:521-530 (1998), which is hereby incorporated by reference in its entirety, proposed that the GADD45-like proteins mediate activation of the p38/JNK pathway, via MTK1, in response to environmental stresses.

### CREB-2 (ATF)

An activating transcription factor (ATF)-binding site is a promoter element present in a wide variety of viral and cellular genes, including ElA-inducible adenoviral genes and cAMP-inducible cellular genes. Hai et al., Genes Dev. 3:2083-2090 (1989), which is hereby incorporated by reference in its entirety, identified cDNAs encoding eight different human ATF consensus-binding proteins, including a partial cDNA corresponding to ATF4. Hai et al. found that members of this family share significant sequence similarity within a leucine zipper DNA-binding motif and an adjacent basic region; the proteins show little similarity outside of these regions.

The cAMP response element (CRE) is an octanucleotide motif that mediates diverse transcriptional regulatory effects. By screening a Jurkat T-cell line expression library for the ability to bind CRE, Karpinski et al., Proc. Nat. Acad. Sci. 89:4820-4824 (1992), which is hereby incorporated by reference in its entirety, isolated and characterized a full-length cDNA corresponding to ATF4, which they called CREB2 (CRE-binding protein-2). The predicted protein contains 351-amino acids. Northern blot analysis revealed that the 1.5-kb CREB2 mRNA was expressed in all human tumor cell lines and mouse organs tested. Unlike CREB, which activates transcription from CRE-containing promoters, CREB-2 functions as a specific repressor of CRE-dependent transcription. The transcriptional repressor activity resides within the C-terminal leucine zipper and basic domain region of the CREB2 protein.

The p40tax gene product of human T-cell leukemia virus type 1 (HTLV-1) activates HTLV-1 viral transcription in trans through tax-responsive enhancers in the long terminal repeats. Tsujimoto et al., J. Virol. 65:1420-1426 (1991), which is hereby incorporated by reference in its entirety, identified ATF4 (CREB2) as TAXREB67, a protein that binds to the tax-responsive enhancer element in HTLV-1.

Tanaka et al., Genes Cells 3:801-810 (1998), which is hereby incorporated by reference in its entirety, used gene targeting to generate mice lacking Atf4 (CREB2). They found that Atf4-deficient mice exhibited severe microphthalmia. The Atf4-deficient eyes revealed a normal gross lens structure up to embryonic day 14.5, after which the lens degenerated due to apoptosis without the formation of lens secondary fiber cells. Retinal development was normal in the mutant mice. The lens-specific expression of Atf4 in the mutant mice led not only to the recovery of lens secondary fibers but also to the induction of hyperplasia of these fibers. Tanaka et al. (1998) concluded that ATF4 is essential for the later stages of lens fiber cell differentiation.

ATF-2 as a homodimer or heterodimer bind to cAMP response element, and overexpression of ATF-2 has been shown to significantly enhance growth rate and proliferation of cells grown under stress conditions (Huguier et al., MCB 18:7020 (1998), which is hereby incorporated by reference in its entirety) and exposed to DNA damaging radiation (Koolj et al., Oncogene 22:4235 (2003), which is hereby incorporated by reference in its entirety). A main protective response of cells to ionizing radiation, UV damage, and DNA damaging factors is induction of cell cycle arrest through the activation of cell cycle check points. This period of quiescence allows the cell to recognize and repair DNA damage. ATF-2 is one of the transcription factors that acts on an essential enzyme in cell cycle arrest, ATM kinase. Because increased BVR expression has been shown to upregulate ATF-2, it is believed that BVR can induce enhanced growth rate and proliferation of cells grown under stress conditions. ATF-2 plays an important role in placenta formation and development of the skeletal as well as the central nervous systems, oncogenic transformation and adaptive response to viral infection and genotoxic stress (Reinhold et al., Nature 379:262 (1996); Maekawa et al., J Biol Chem 274:17813 (1999); Liu and Green Cell 61:1217 (1990); VanDam and Castellazzi, Oncogene 202:453 (2001); VanDam et al., EMBO J 14:1798 (1995), which are hereby incorporated by reference in their entirety).

### IAP-1/IAP-2

By testing hybrids containing various deletions of chromosome 3, Miller et al., Am. J. Hum. Genet. 41:1061-1070 (1987), which is hereby incorporated by reference in its entirety, described an IgM monoclonal antibody, 1D8, that recognized an antigen coded by a gene located in the region 3cen-q22. The monoclonal antibody was designated MER6. The antigen was absent in the Rh deficiency syndrome, Rh-null hemolytic anemia. This antigen probably had no pathogenetic role in the Rh deficiency, which was shown by Cherif-Zahar et al., Nature Genet. 12:168-173 (1996), which is hereby incorporated by reference in its entirety, to be due to mutation in the Rh50 gene on chromosome 6. They noted that many cell membrane components are missing from the multisubunit Rh complex when the RH50A gene is mutant.

Integrin-associated protein (IAP) is a 50-kD membrane protein with an amino-terminal immunoglobulin domain and a carboxyl-terminal multiple-membrane-spanning region. It is involved in the increase in intracellular calcium concentration that occurs upon cell adhesion to extracellular matrix. IAP is also expressed on erythrocytes, which have no known integrins. IAP is identical to OA3, an ovarian carcinoma antigen (Mawby et al., Biochem. J. 304:525-530 (1994), which is hereby incorporated by reference in its entirety). Lindberg et al., J. Biol. Chem. 269:1567-1570 (1994), which is hereby incorporated by reference in its entirety, showed that IAP expression is reduced on Rh(null) erythrocytes. By fluorescence in situ hybridization they showed that the IAP structural gene maps to 3q13.1-q13.2, within a region known to contain a gene encoding the Rh-associated 1D8 antigen. By expression studies on human erythrocytes and IAP transfectants, IAP was shown to be identical to the 1D8 antigen and to CD47, a cell surface protein with broad tissue distribution, reduced in expression on Rh(null) erythrocytes. Lindberg et al. (1994) stated that these studies demonstrated an unexpected link between integrin signal transduction and erythrocyte membrane structure.

### P16(INK4)

Cyclin-dependent kinase inhibitor-2A (CDKN2A) goes by the colloquial designation p16, which is sometimes referred to as p16(INK4). The gene was originally symbolized MTS1 (for multiple tumor suppressor-1) by Kamb et al., Science 264: 436-440 (1994), which is hereby incorporated by reference in its entirety, who later used the symbol CDKN2 because MTS1 had been preempted by the malignant transformation suppression-1 gene located on 1p.

Chromosome region 9p21 is involved in chromosomal inversions, translocations, heterozygous deletions, and homozygous deletions in a variety of malignant cell lines including those from glioma, nonsmall cell lung cancer, leukemia, and melanoma. Deletion of 9p21 markers is found in more than half of all melanoma cell lines. These findings suggest that 9p21 contains a tumor suppressor locus that may be involved in the genesis of several tumor types. Kamb et al., Science 264:436-440 (1994), which is hereby incorporated by reference in its entirety, localized a putative tumor suppressor locus to band 9p21 in a region of less than 40 kb by means of analyzing homozygous deletions in melanoma cell lines. The region was found to contain a gene, called MTS1 (for multiple tumor suppressor-1), that encodes a previously identified inhibitor (p16) of cyclin-dependent kinase-4 (CDK4). The sequence of the MTSl gene as determined by Kamb et al. (1994) was identical to that of the p16 gene as determined by Serrano et al., Nature 366:704-707 (1993), which is hereby incorporated by reference in its entirety. MTS1 was found to be homozygously deleted at high frequency in cell lines derived from tumors of lung, breast, brain, bone, skin, bladder, kidney, ovary, and lymphocyte. Melanoma cell lines carried at least one copy of MTS1 in combination with a deleted allele. Melanoma cell lines that carried at least 1 copy of MTS1 frequently showed nonsense, missense, or frameshift mutations in the gene. Thus, MTS1 may rival p53 in the universality of its involvement in tumorigenesis. Furthermore, it illustrates, as does p53, the relationship between the tumor suppressor genes and the regulation of the cell cycle.

The p16 gene (CDKN2A) was mapped to 9p21(Kamb et al. (1994); Nobori et al., Nature 368:753-756 (1994), which are hereby incorporated by reference in their entirety). This same region has frequently been involved in deletions and rearrangements in dysplastic nevi (Cowen et al., J. Nat. Cancer Inst. 80: 1159-1164 (1988), which is hereby incorporated by reference in its entirety), a major precursor lesion of melanoma, and in cutaneous malignant melanoma, or CMM (Fountain et al., Proc. Nat. Acad. Sci. 89:10557-10561 (1992), which is hereby incorporated by reference in its entirety), and was shown by Petty et al., Am. J. Hum. Genzet. 53:96-104 (1993), which is hereby incorporated by reference in its entirety, to be involved in a constitutional deletion in a patient with multiple primary melanomas. A gene for familial malignant melanoma, symbolized CMM2, has been mapped to 9p21.

The frequent deletion or mutation of CDKN2A in tumor cells suggests that p16 acts as a tumor suppressor. Lukas et al., Nature 375:503-506 (1995), which is hereby incorporated by reference in its entirety, showed that wildtype p16 arrests normal diploid cells in late G1, whereas a tumor-associated mutant of pl 6 does not. Significantly, the ability of p16 to induce cell cycle arrest was lost in cells lacking functional retinoblastoma protein. Thus, loss of p16, overexpression of D-cyclins, and loss of retinoblastoma have similar effects on G1 progression, and may represent a common pathway to tumorigenesis. The mutation used by Lukas et al. (1995) in their studies was a C-to-T transition changing proline-114 to leucine and had been observed in 3 independent melanoma cell lines. Koh et al., Nature 375:506-510 (1995), which is hereby incorporated by reference in its entirety, reported similar results. They demonstrated that p16 can act as a potent and specific inhibitor of progression through the G1 phase of the cell cycle and that several tumor-derived alleles of p16 encode functionally compromised proteins. In vivo, the presence of functional retinoblastoma protein appeared to be necessary but may not be sufficient to confer full sensitivity to p16-mediated growth arrest. In addition to the P114L allele, they used an asp74-to-asn (D74N) mutant, a de novo somatic mutation isolated independently from tumors of the esophagus and bladder; an asp84-to-asn (D84N) mutation found in a survey of esophageal squamous cell carcinomas; and several other mutations associated with melanoma.

Stott et al., EMBO J. 17:5001-5014 (1998), which is hereby incorporated by reference in its entirety, stated that the alpha transcript of CDKN2A has been shown to encode p16(INK4a), a recognized tumor suppressor that induces a G1 cell cycle arrest by inhibiting the phosphorylation of the Rb protein by the cyclin-dependent kinases CDK4 and CDK6. The beta transcript of CDKN2A encodes p14(ARF). The predicted 132-amino acid p14(ARF) is shorter than the corresponding mouse protein, p19(ARF), and the 2 proteins share only 50% identity. However, both proteins have the ability to elicit a p53 response, manifest in the increased expression of both CDKN1A and MDM2, and resulting in a distinctive cell cycle arrest in both the G1 and G2/M phases. Zhange et al., Cell 92: 25-734 (1998), which is hereby incorporated by reference in its entirety, stated that the 2 unrelated proteins encoded by the INK4A-ARF locus function in tumor suppression. Zhange et al. (1998) showed that ARF binds to MDM2 and promotes the rapid degradation of MDM2. This interaction is mediated by the E1-beta-encoded N-terminal domain of ARF and a C-terminal region of MDM2. ARF-promoted MDM2 degradation is associated with MDM2 modification and concurrent p53 stabilization and accumulation. The functional consequence of ARF-regulated p53 levels via MDM2 proteolysis is evidenced by the ability of ectopically expressed ARF to restore a p53-imposed G1 cell cycle arrest that is otherwise abrogated by MDM2. Thus, Zhang et al. (1998) concluded that deletion of the ARF-INK4A locus simultaneously impairs the INK4A--cyclin D/CDK4--RB and the ARF--MDM2--p53 pathways.

Igaka et al., Biochem. Biophys. Res. Commun. 203:1090-1095 (1994), which is hereby incorporated by reference in its entirety, found homozygous deletion of p16 in 12 of 13 esophageal cancer cell lines and in 2 of 9 gastric cancer cell lines. They also found that p16 gene loss, cyclin D1, and p53 gene mutations occurred independently in these cell lines. They interpreted these results as indicating that changes in the p16 gene are involved in most esophageal cancers and play a critical role in the development of this type of malignancy.

Liu et al., Oncogene 11:405-412 (1995), which is hereby incorporated by reference in its entirety, described a family with inherited melanoma in which a novel mutation in exon 2 of the p16(INK4A) gene segregated with disease. The mutant allele encoded a protein with an in-frame deletion of 2 amino acids (asp96 and leu97). They showed that the mutant protein is functionally abnormal: it was unable to bind CDK4 in vitro and did not inhibit colony formation in tertiary passage rat embryo fibroblasts. Moreover, in a metastatic lesion from 1 patient, the wildtype allele was deleted and the mutant allele retained. Liu et al. (1995) concluded that family members carrying the germline mutation in this gene are predisposed to melanoma.

Pilon et al., J. Clin. Endocr. Metab. 84:2776-2779 (1999), which is hereby incorporated by reference in its entirety, investigated inactivation of the p1 6 tumor suppressor gene in a series of 14 adrenocortical tumors. Using 11 polymorphic microsatellite markers spanning the short arm of chromosome 9, they demonstrated that 3 of 7 adrenocortical carcinomas and 1 of 7 adrenocortical adenomas had LOH within chromosome 9p21, the region containing p16. Immunohistochemistry showed the absence of p16 nuclear staining in all adrenocortical tumors with LOH within 9p21, and positive staining in all remaining tumors without LOH. The authors concluded that LOH within 9p21 associated with lack of p16 expression occurs in a considerable proportion of adrenocortical malignant tumors but is rare in adenomas. Furthermore, they suggested that inactivation of p16 may contribute to the deregulation of cell proliferation in this neoplastic disease.

The p16(INK4A) cyclin-dependent kinase inhibitor is implicated in replicative senescence, the state of permanent growth arrest provoked by cumulative cell divisions or as a response to constitutive Ras-Raf-MEK signaling in somatic cells. Ohtani et al., Nature 409:1067-1070 (2001), which is hereby incorporated by reference in its entirety, demonstrated a role for the ETS1 and ETS2 transcription factors in regulating the expression of p16(INK4A) in these different contexts based on their ability to activate the p16(INK4A) promoter through an ETS binding site and their patterns of expression during the lifespan of human diploid fibroblasts. The induction of p16(INK4A) by ETS2, which is abundant in young human diploid fibroblasts, is potentiated by signaling through the Ras-Raf-MEK kinase cascade and inhibited by a direct interaction with the helix-loop-helix protein ID1. In senescent cells, where the ETS2 levels and MEK signaling decline, the marked increase in p16(INK4A) expression is consistent with the reciprocal reduction of ID1 and accumulation of ETS1.

### P27(KIP1)

Stegmaier et al., Blood 86:38-44 (1995), which is hereby incorporated by reference in its entirety, studied loss of heterozygosity (LOH) in the region 12p13-p12 in acute lymphoblastic leukemia (this chromosomal region often shows deletion in such patients). In 15% of informative patients, there was evidence of LOH of the TEL locus which was not evident on cytogenetic analysis. Detailed examination of patients with LOH showed that the critically deleted region included a second candidate tumor suppressor gene, referred to by them as KIP1, which encodes the cyclin-dependent kinase inhibitor previously called p27 (Toyoshima and Hunter, Cell 78:67-74 (1994) and Polyak et al., Cell 78:59-66 (1994), which are hereby incorporated by reference in their entirety,). Based on the STS content of TEL-positive YACs, Stegmaier et al. (1995) reported that KIP1 and TEL were in close proximity.

Cyclin-dependent kinase (CDK, e.g., CDK2) activation requires association with cyclins (e.g., CCNE1) and phosphorylation by CAK (CCNH), and leads to cell proliferation. hIhibition of cellular proliferation occurs upon association of CDK inhibitor (e.g., CDKN1B) with a cyclin-CDK complex. Sheaff et al., Genes Dev. 11:1464-1478 (1997), which is hereby incorporated by reference in its entirety, showed that expression of CCNE1-CDK2 at physiologic levels of ATP results in phosphorylation of CDKN1B at thr187, leading to elimination of CDKN1B from the cell and progression of the cell cycle from G1 to S phase. At low ATP levels, the inhibitory functions of CDKN1B are enhanced, thereby arresting cell proliferation.

Apoptosis of human endothelial cells after growth factor deprivation is associated with rapid and dramatic upregulation of cyclin A-associated CDK2 activity. Levkau et al., Molec. Cell 1:553-563 (1998), which is hereby incorporated by reference in its entirety, showed that in apoptotic cells the carboxyl-termini of the CDK inhibitors CDKN1A and CDKN1B are truncated by specific cleavage. The enzyme involved in this cleavage is CASP3 and/or a CASP3-like caspase. After cleavage, CDKN1A loses its nuclear localization sequence and exits the nucleus. Cleavage of CDKN1A and CDKN1B resulted in a substantial reduction in their association with nuclear cyclin-CDK2 complexes, leading to a dramatic induction of CDK2 activity. Dominant-negative CDK2, as well as a mutant CDKN1A resistant to caspase cleavage, partially suppressed apoptosis. These data suggested that CDK2 activation, through caspase-mediated cleavage of CDK inhibitors, may be instrumental in the execution of apoptosis following caspase activation.

High levels of p27(KIP1), present in quiescent (G0) cells, have been shown to decline upon mitogen induction (Sherr and Roberts, Genes Dev. 9: 1149-1163 (1995), which is hereby incorporated by reference in its entirety). Braun-Dullaeus, J. Clin. Invest. 104:815-823 (1999), which is hereby incorporated by reference in its entirety, explored the role of p27(KIP1) and other cell cycle proteins in mediating angiotensin II-induced vascular smooth muscle cell hypertrophy or hyperplasia. Angiotensin II treatment (100 nM) of quiescent vascular smooth muscle cells led to upregulation of the cell cycle regulatory proteins cyclin D1, CDK2, proliferating cell nuclear antigen, and CDK1. Levels of p27(KIP1), however, remained high, and the activation of the G1-phase CDK2 was inhibited as the cells underwent hypertrophy. Angiotensin II stimulated an increase in [(3)H]thymidine incorporation and the percentage of S-phase cells in p27(KIP1) antisense oligodeoxynucleotide (ODN)-transfected cells but not in control ODN transfected cells. The authors concluded that angiotensin II stimulation of quiescent cells in which p27(KIP1) levels are high results in hypertrophy but promotes hyperplasia when levels of p27(KIP1) are low, as in the presence of other growth factors.

Medema et al., Nature 404:782-787 (2000), which is hereby incorporated by reference in its entirety, demonstrated that p27(KIP1) is a major target of AFX-like forkhead proteins. They demonstrated that AFX integrates signals from PI3K/PKB signaling and RAS/RAL signaling to regulate transcription of p27(KIP1). They demonstrated that p27−/− cells are significantly less inhibited by AFX activity than their p27+/+ counterparts.

Peters and Ostrander, Nature Genet. 27:134-135 (2001), which is hereby incorporated by reference in its entirety, commented on the work of Di Cristofano et al., Nature Genet. 27:222-224 (2001), which is hereby incorporated by reference in its entirety, demonstrating how cooperation between Cdkn1b and Pten contribute to suppression of prostate tumors. They gave a useful tabulation of the cytogenetic location of 16 mapped prostate cancer susceptibility loci and candidate genes.

Phosphorylation leads to the ubiquitination and degradation of CDKN1B. Carrano et al., Nature Cell Biol. 1: 193-199 (1999), which is hereby incorporated by reference in its entirety, determined that SKP2 specifically recognizes phosphorylated CDKN1B predominantly in S phase rather than in G1 phase, and is the ubiquitin-protein ligase necessary for CDKN1B ubiquitination.

Shin et al., Nature Med. 8:1145-1152 (2000), which is hereby incorporated by reference in its entirety, demonstrated a novel mechanism of AKT-mediated regulation of p27(KIP1). Blockade of HER2/NEU in tumor cells inhibited AKT kinase activity and upregulated nuclear levels of p27(KIP1). Recombinant AKT and AKT precipitated from tumor cells phosphorylated wildtype p27 in vitro. P27 contains an AKT consensus RXRXXT(157)D within its nuclear localization motif. Active (myristoylated) AKT phosphorylated wildtype p27 in vivo but was unable to phosphorylate a T157A-p27 mutant. Wildtype p27 localized in the cytosol and nucleus, whereas the mutant p27 localized exclusively in the nucleus and was resistant to nuclear exclusion by AKT. Expression of phosphorylated AKT in primary human breast cancers statistically correlated with the expression of p27 in tumor cytosol. Shin et al. (2002), which is hereby incorporated by reference in its entirety, concluded that AKT may contribute to tumor cell proliferation by phosphorylation and cytosolic retention of p27, thus relieving CDK from p27-induced inhibition.

Liang et al., Nature Med. 8:1153-1160 (2002), which is hereby incorporated by reference in its entirety, demonstrated that AKT phosphorylates p27, impairs the nuclear import of p27, and opposes cytokine-mediated G1 arrest. In cells transfected with constitutively active AKT, wildtype p27 mislocalized to the cytoplasm, but mutant p27 was nuclear. In cells with activated AKT, wildtype p27 failed to cause G1 arrest, while the antiproliferative effect of the mutant p27 was not impaired. Cytoplasm p27 was seen in 41% (52 of 128) primary human breast cancers in conjunction with AKT activation and was correlated with a poor patient prognosis. Liang et al. (2002) concluded that their data showed a novel mechanism whereby AKT impairs p27 function that is associated with an aggressive phenotype in human breast cancer.

Viglietto et al., Nature Med. 8:1136-1144 (2002), which is hereby incorporated by reference in its entirety, independently demonstrated that AKT regulates cell proliferation in breast cancer cells by preventing p27(KIP1)-mediated growth arrest. They also showed that threonine at position 157 is an AKT phosphorylation site and causes retention of p27(KIP1) in the cytoplasm, precluding p27(KIP1)-induced G1 arrest.

Fero et al., Cell 85:733-744 (1996), which is hereby incorporated by reference in its entirety, found that targeted disruption of the murine p27(Kip1) gene caused a gene dose-dependent increase in animal size without other gross morphologic abnormalities. All tissues were enlarged and contained more cells, although endocrine abnormalities were not evident. Thymic hyperplasia was associated with increased T-lymphocyte proliferation, and T cells showed enhanced IL2 responsiveness in vitro. Thus, p27 deficiency may cause a cell-autonomous defect resulting in enhanced proliferation in response to mitogens. In the spleen, the absence of p27 selectively enhanced proliferation of hematopoietic progenitor cells. That p27 and Rb function in the same regulatory pathway was suggested by the finding that p27 deletion, like deletion of the Rb gene, uniquely caused neoplastic growth of the pituitary pars intermedia. The absence of p27 also caused an ovulatory defect and female sterility. Maturation of second ovarian follicles into corpora lutea, which express high levels of p27, was markedly impaired.

Zindy et al., Proc. Nat. Acad. Sci. 96:13462-13467 (1999), which is hereby incorporated by reference in its entirety, generated mice with targeted deletions of both the Ink4d and Kip1 genes. They found that terminally differentiated, postmitotic neurons in these mice reentered the cell cycle, divided, and underwent apoptosis. Zindy et al. (1999) noted that when either Ink4d or Kip1 alone are deleted, the postmitotic state is maintained, suggesting a redundant role for these genes in mature neurons.

Mitsuhashi et al., Proc. Nat. Acad. Sci. 98:6435-6440 (2001), which is hereby incorporated by reference in its entirety, described a mouse model in which p27(Kip1) transgene expression was spatially restricted to the central nervous system neuroepithelium and temporally controlled with doxycycline. Transgene-specific transcripts were detectable within 6 hours of doxycycline administration, and maximum nonlethal expression was approached within 12 hours. After 18 to 26 hours of transgene expression, the G1 phase of the cell cycle was estimated to increase from 9 to 13 hours in the neocortical neuroepithelium, the maximum G1 phase length attainable in this proliferative population in normal mice. Thus, the data established a direct link between p27(Kip1) and control of G1 phase length in the mammalian central nervous system and unveiled intrinsic mechanisms that constrain the G1 phase length to a putative physiologic maximum despite ongoing p27(Kip1) transgene expression.

Phosphorylation of p27(Kip1) on threonine-187 by CDK2 is thought to initiate the major pathway for p27 proteolysis. To critically test the importance of this pathway in vivo, Malek et al., Nature 413: 323-327 (2001), which is hereby incorporated by reference in its entirety, replaced the murine p27 gene with one that encoded alanine instead of threonine at position 187. Malek et al. (2001) demonstrated that cells expressing p27 with the T187A change were unable to downregulate p27 during the S and G2 phases of the cell cycle, but that this had a surprisingly modest effect on cell proliferation both in vitro and in vivo. Malek et al. (2001) demonstrated a second proteolytic pathway for controlling p27, one that is activated by mitogens and degrades p27 exclusively during G1.

### P18 (CDK4 Inhibitor)

Cyclin-dependent kinase inhibitors (CKIs) are a group of low molecular weight proteins that associate with cyclin-CDK complexes or CDKs alone and inhibit their activity. Members of the INK4 family of CKIs, which includes CDKN2C, specifically bind and inhibit CDK4 and CDK6, thereby preventing cyclin D-dependent phosphorylation of RB1.

By using a yeast 2-hybrid screen to search for CDK6-interacting proteins, Guan et al., Genes Dev. 8:2939-2952 (1994), which is hereby incorporated by reference in its entirety, isolated a partial cDNA encoding a protein that they designated p18 based on its molecular mass of 18 kD. They used the partial cDNA to screen a HeLa cell library and recovered additional cDNAs corresponding to the entire p18 coding region. Sequence analysis revealed that the predicted 168-amino acid p18 protein shares 38% and 42% sequence identity with p16/INK4A and p14/INK4B, respectively. Like p14 and p16, p18 contains an ankyrin repeat domain. Using Northern blot analysis, Guan et al. (1994) found that p18 is expressed as multiple transcripts in various human tissues, with the strongest expression in skeletal muscle.

Guan et al., Genes Dev. 8:2939-2952 (1994), which is hereby incorporated by reference in its entirety, showed that, both in vivo and in vitro, p18 interacted strongly with CDK6 and weakly with CDK4, but not with the other CDKs tested. Recombinant p18 inhibited the kinase activity of cyclin D-CDK6 in vitro. Ectopic expression of either p16 or p18 suppressed the growth of human cells in a manner that appears to correlate with the presence of a wildtype RB1 function. By fluorescence in situ hybridization, Guan et al. (1994) mapped the p18 gene to 1p32, a chromosomal region associated with abnormalities in a variety of human tumors.

Lapointe et al., Cancer Res. 56:4586-4589 (1996), which is hereby incorporated by reference in its entirety, identified a single amino acid substitution (ala72 to pro; A72P) in BT-20 human breast cancer cells that abrogated the ability of p18 to interact with CDK6 and to suppress cell growth. These authors suggested that p18 inactivation by point mutations may contribute to deregulated growth control in certain cell lines and/or tumors. Blais et al., Biochem. Biophys. Res. Commun. 247:146-153 (1998), which is hereby incorporated by reference in its entirety, found this p18 variant in 3 of 35 breast tumors examined, and suggested that it may be a polymorphism.

Bai et al., Molec. Cell. Biol 23:1269-1277 (2003), which is hereby incorporated by reference in its entirety, noted that targeted disruption of Ink4c in mice leads to spontaneous pituitary tumors and lymphomas later in life. Treatment of Inc4c null and heterozygous mice with a chemical carcinogen resulted in tumor development at an accelerated rate. Bai et al. (2003) concluded that, since the remaining wildtype allele of Ink4c was neither mutated nor silenced in tumors derived from heterozygotes, Ink4c is a haploinsufficient tumor suppressor in mice.

### FASN

Fatty acid synthase (“FASN”) catalyzes the conversion of acetyl-CoA and malonyl-CoA, in the presence of NADPH, into long-chain saturated fatty acids (Wakil, Biochemistiy 28:4523-4530 (1989), which is hereby incorporated by reference in its entirety. In prokaryotes and plants, FASN consists of an acyl carrier protein and 7 structurally independent monofunctional enzymes. In animals, however, all of the component enzymatic activities of FASN and acyl carrier protein are organized in one large polypeptide chain.

Jayakumar et al., Proc. Nat. Acad. Sci. USA 92:8695-8699 (1995), which is hereby incorporated by reference in its entirety, isolated and sequenced cDNA clones representing the 2 ends of the human FASN gene and also isolated overlapping genomic clones from human YAC libraries. By fluorescence in situ hybridization, they mapped the FASN gene to 17q25. Southern analyses suggested that a single 40-kb cosmid clone encompasses the entire coding region of the gene.

Jayakumar et al., Genomics 23: 420-424 (1994), which is hereby incorporated by reference in its entirety, purified fatty acid synthase to near homogeneity from a human hepatoma cell line, HepG2. The specific activity of the enzyme was found to be half that of chicken liver enzyme. They also cloned the human brain FASN cDNA. The cDNA sequence had an open reading frame of 7,512 bp that encoded a 2504-amino acid protein with relative mass of 272,516. The amino acid sequence of the human enzyme had 79% and 63% identity, respectively, with the sequences of the rat and chicken enzymes. Northern analysis revealed that human FASN mRNA is about 9.3 kb in size and that its level varies among human tissues, with brain, lung, and liver tissues showing prominent expression. Sequence variants of unknown origin and significance were found in the enzyme derived from HepG2.

Ye et al., Biochim. Biophys. Acta 1493:373-377 (2000), which is hereby incorporated by reference in its entirety, investigated the expression of ESR1 in prostate cancer cell lines and unexpectedly found a FASN/ESR1 fusion transcript. Using semi-nested RT-PCR analysis of ESR1 and its variants, Ye et al., (2000) found that the N-terminal coding region of FASN containing domain 1 was fused to the C-terminal coding region of the ESR1 ligand binding domain. Nested RT-PCR also detected the fusion transcript in breast, cervical, and bladder cancer cell lines.

Loftus et al., Science 288:2379-2381 (2000), which is hereby incorporated by reference in its entirety, identified a link between anabolic energy metabolism and appetite control. Both systemic and intracerebroventricular treatment of mice with fatty acid synthase inhibitors (cerulenin and C75, a synthetic compound) led to inhibition of feeding and dramatic weight loss. C75 inhibited expression of the prophagic signal neuropeptide Y in the hypothalamus and acted in a leptin-independent manner that appears to be mediated by malonyl-CoA. Loftus et al. (2000) suggested that FASN may represent an important link in feeding regulation and may be a potential therapeutic target for obesity.

In animals, including humans, the source of long chain saturated fatty acids is either de novo synthesis, which is mediated by fatty acid synthase, ingested food, or both. To understand the importance of de novo fatty acid synthesis, Chirala et al., Proc. Nat. Acad. Sci. 100:6358-6363 (2003), which is hereby incorporated by reference in its entirety, generated FASN knockout mice. The heterozygous mutant mice were ostensibly normal; however, levels of FASN mRNA and activity were approximately 50% and 35% lower, respectively, than those of wildtype mice. When the heterozygous mutant mice were interbred, no null mice were produced; thus, FASN is essential during embryonic development. Furthermore, the number of heterozygous progeny was 70% less than predicted by Mendelian inheritance, indicating partial haploid insufficiency. Even when 1 parent was wildtype and the other heterozygous, the estimated loss of heterozygous progeny was 60%. Most of the FASN-null embryos died before implantation and the heterozygous embryos died at various stages of development. Feeding the breeders a diet rich in saturated fatty acids did not prevent the loss of homo- or heterozygotes.

### Cyclin A

Wang et al., Nature 343:555-557 (1990), which is hereby incorporated by reference in its entirety, cloned a single hepatitis B virus integration site in a human hepatocellular carcinoma at an early stage of development, and also cloned its germline counterpart. The normal locus was found to be transcribed into 2 polyadenylated mRNA species of 1.8 and 2.7 kb. Wang et al. (1990) isolated a cDNA clone from a normal adult human liver that had an open reading frame with a coding capacity for a protein of 432 amino acids and relative molecular mass of 48,536. Strong homologies in amino acid sequence identified the protein as a human cyclin A. The HBV integration was found to have occurred within an intron. Wang et al. (1990) suggested that disruption of the cyclin A gene by viral insertion was responsible for tumorigenesis.

Cyclins are highly conserved proteins associated with proliferating cells. They show a steady accumulation throughout interphase until the G2/M transition, followed by rapid disappearance at the onset of anaphase. They are highly conserved in evolution, having been identified in yeast, clam, starfish, sea urchin, and Drosophila. Two groups of cyclins, A and B, are distinguished on the basis of their sequence and pattern of accumulation during the cell cycle. Both cyclins will complex with and activate the serine-threonine kinase p34(cdc2) during the G2/M phase transition. Cyclins are also referred to as proliferating cell nuclear antigens. Nonrandom integration of HBV in hepatocellular carcinoma has been related to chromosome 11 and to chromosome 4. Furthermore, interruption of the coding region of the gene for retinoic acid receptor beta by viral DNA has been reported. By in situ hybridization, Blanquet et al., Genomics 8:595-597 (1990), which is hereby incorporated by reference in its entirety, mapped the CCNA gene to 4q26-q27. They pointed to the interest of this finding in connection with the demonstrated loss of heterozygosity for markers on 4q in tumor tissue of patients with liver cancer (Buetow et al., Proc. Nat. Acad. Sci. 86: 8852-8856 (1989), which is hereby incorporated by reference in its entirety).

Girard et al., Cell 67:1169-1179 (1991), which is hereby incorporated by reference in its entirety, showed that cyclin A protein is synthesized and localized into the nucleus at the onset of S phase in nontransformed mammalian fibroblasts. Inhibition of cyclin A synthesis or activity through microinjection of plasmids encoding antisense cyclin A cDNA or affinity-purified anti-cyclin A antibodies during G1 phase abolished the nuclear staining for cyclin A and inhibited DNA synthesis. No similar effect was observed with injection of other antisense vectors, including antisense cyclin B. Girard et al. (1991) suggested that cyclin A plays a major role in the control of DNA replication. Henglin et al., Proc. Nat. Acad. Sci. 91:5490-5494 (1994), which is hereby incorporated by reference in its entirety, cloned and sequenced the human CCNA gene and cDNAs representing its mRNAs and characterized its promoter. Using synchronized cultures of NIH 3T3 cells stably transfected with cyclin A promoter/luciferase constructs, they showed that the promoter is repressed during the G1 phase of the cell cycle and is activated at S-phase entry. Cell cycle regulation of the CCNA promoter is mediated by sequences extending from −79 to +100 relative to the predominant transcription start site. The presence of a functional retinoblastoma protein is not required.

The mammalian A-type cyclin family consists of 2 members, cyclin A1 and cyclin A2. Cyclin A2 promotes both G1/S and G2/M transitions (Pagano et al., EMBO J. 11:961-971 (1992). Murphy et al., Nature Genet. 15:83-86 (1997), which is hereby incorporated by reference in its entirety), demonstrated that a targeted deletion of the murine Ccna2 gene is embryonically lethal, although homozygous null mutant embryos developed normally until postimplantation, approximately day 5.5 postcoitum. The authors suggested that the embryos survived either because a maternal pool of cyclin A2 protein persists until at least the blastocyst stage, or because cyclin A1 plays an unexpected role during early embryo development. Cyclin A1 is expressed in mice exclusively in the germline lineage (Sweeney et al., Development 122: 53-64 (1996), which is hereby incorporated by reference in its entirety, and is expressed in humans at highest levels in the testis and certain myeloid leukemia cells (Yang et al., Cancer Res. 57: 913-920 (1997), which is hereby incorporated by reference in its entirety).

### Cyclin E1

Koff et al., Cell 66:1217-1228 (1991), which is hereby incorporated by reference in its entirety, isolated a new human cyclin, named cyclin E, by complementation of a triple cln deletion in Saccharomyces cerevisiae. Cyclin E showed genetic interactions with the CDK18 gene, suggesting that it functions at START by interacting with the CDK18 protein. Two human genes were identified that could interact with cyclin E to perform START in yeast containing a cdc28 mutation. One was CDK1-HS, and the second was the human homolog of Xenopus CDK2. Keyomarsi et al., Cancer Res. 54:380-385 (1994), which is hereby incorporated by reference in its entirety, demonstrated that breast cancers, as well as some other solid tumors, show severe quantitative and qualitative alterations in cyclin E protein production. In breast cancer, the alterations in cyclin E expression became progressively worse with increasing stage and grade of the tumor, suggesting its potential use as a prognostic marker.

Cyclin-dependent kinase (CDK) activation requires association with cyclins (e.g., Cyclin E1) and phosphorylation by CAK, and leads to cell proliferation. Inhibition of cellular proliferation occurs upon association of CDK inhibitor (e.g., CDKN1B) with a cyclin-CDK complex. Sheaff et al., Genes Dev. 11: 1464-1478 (1997), which is hereby incorporated by reference in its entirety, showed that expression of Cyclin E1-CDK2 at physiologic levels of ATP results in phosphorylation of CDKN1B at thr187, leading to elimination of CDKN1B from the cell and progression of the cell cycle from G1 to S phase. At low ATP levels, the inhibitory functions of CDKN1B are enhanced, thereby arresting cell proliferation.

Keyomarsi et al., New Eng. J. Med. 347:1566-1575 (2002), which is hereby incorporated by reference in its entirety, investigated cyclin E as a determinant of the virulence and metastatic potential of breast cancer cells. In normal dividing cells, cyclin E regulates the transition from the G1 phase to the S phase, and a high level of cyclin E protein accelerates the transition through the G1 phase. They assayed for cyclin E in tumor tissue from 395 patients with breast cancer and correlated the findings with follow-up (median 6.4 years). Levels of total cyclin E and low-molecular weight cyclin E in tumor tissue, as measured by Western blot assay, correlated strongly with survival in patients with breast cancer. The hazard ratio for death from breast cancer for patients with high total cyclin E levels as compared with those with low total cyclin E levels was 13.3, or about 8 times as high as the hazard ratios associated with other independent clinical and pathologic risk factors.

Cyclins bind to and activate cyclin-dependent kinases (CDKs) to form serine/threonine kinase holoenzyme complexes that regulate the eukaryotic cell cycle. Cyclins A, D and E are required for mammalian cells to traverse G1 and enter S phase. Cyclin E controls the initiation of DNA synthesis by activating CDK2; the KIP1 and CIP1 proteins bind and inhibit cyclin E-CDK complexes. By searching an EST database with a cyclin box consensus sequence, Gudas et al., Molec. Cell. Biol. 19:612-622 (1999), which is hereby incorporated by reference in its entirety, identified rat and mouse cDNAs encoding cyclin E2. They carried out additional EST database searches and performed RACE to identify human cyclin E2 cDNAs. Northern blot analysis revealed that the 2.8-kb cyclin E2 mRNA is expressed in several normal human tissues, with the highest levels in testis, thymus, and brain. The level of cyclin E2 transcript was consistently elevated in tumor-derived cells compared to nontransformed proliferating cells. Like cyclin E1, the human cyclin E2 gene complemented a Gi cyclin defect in S. cerevisiae. Sequence analysis indicated that the predicted 404-amino acid cyclin E2 protein contains a cyclin box motif and is 47% identical to cyclin E1 (CCNE1).

When expressed in mammalian cells, epitope-tagged cyclin E2 protein localized to the nucleus. The expressed protein associated with CDK2 in a functional kinase complex that was inhibited by both KIP1 and CIP1. Gudas et al. (1999) demonstrated that the catalytic activity associated with cyclin E2 complexes is cell cycle-regulated and peaks at the G1/S transition. Overexpression of either cyclin E1 or cyclin E2 in mammalian cells accelerated G1, indicating that, like cyclin E1, cyclin E2 may be rate-limiting for G1 progression. These authors concluded that multiple unique cyclin E-CDK complexes may regulate cell cycle progression. The researchers also isolated an alternatively spliced human cDNA encoded cyclin E2(SV), a protein missing 45 amino acids within the cyclin box domain. RNase protection assays confirmed that the cyclin E2(SV) mRNA is expressed in normal human thymus. The shorter cyclin E2(SV) isoform did not bind CDK2.

### CDK15A

The human CDK15 tyrosine phosphatases trigger activation of CDK1 by removing inhibitory phosphate from tyrosine and threonine residues of the cyclin-dependent kinases. Thus, the genes encoding these phosphatases are suspected of being potential oncogenes because of their role in promoting cell division. Three human CDK15 genes have been identified: CDK15A, CDK15B, and CDK15C. Demetrick and Beach, Genomics 18:144-147 (1993), which is hereby incorporated by reference in its entirety, mapped the CDK15A gene to 3p21 by fluorescence in situ hybridization with confirmation by PCR analysis of hamster/human somatic cell hybrid DNAs. An area near 3p21 is frequently involved in karyotypic abnormalities in renal carcinomas, small cell carcinomas of the lung, and benign tumors of the salivary gland.

Galaktionov et al., Science 269:1575-1577 (1995), which is hereby incorporated by reference in its entirety, showed that in rodent cells, human CDK15A or CDK15B but not CDK15C phosphatases cooperate with either the gly12-to-val mutation of the HRAS gene or loss of RB1 in oncogenic focus formation. The transformants were highly aneuploid, grew in soft agar, and formed high-grade tumors in nude mice. Overexpression of CDK15B was detected in 32% of human primary breast cancers tested.

CDK15 phosphatases activate the cell division kinases throughout the cell cycle. Fauman et al., Cell 93:617-625 (1998) determined the 2.3-angstrom structure of the human CDK15A catalytic domain. The crystal structure revealed a small alpha/beta domain with a fold unlike previously described phosphatase structures but identical to rhodanese, a sulfur-transfer protein. Only the active-site loop, containing the cys-(X)-5-arg motif, showed similarity to the tyrosine phosphatases. In some crystals, the catalytic cys430 formed a disulfide bond with the invariant cys384, suggesting that CDK15 may be self-inhibited during oxidative stress. Asp383, previously proposed to be the general acid, instead serves a structural role, forming a conserved buried salt bridge. Fauman et al. (1998) proposed that glu431 may act as a general acid.

To protect genome integrity and ensure survival, eukaryotic cells exposed to genotoxic stress cease proliferating to provide time for DNA repair. Mailand et al., Science 288:1425-1429 (2000), which is hereby incorporated by reference in its entirety, demonstrated that human cells respond to ultraviolet light or ionizing radiation by rapid, ubiquitin- and proteosome-dependent protein degradation of CDK15A, a phosphatase that is required for progression from G1 to S phase of the cell cycle. This response involved activated CHK1 protein kinase but not the p53 pathway, and the persisting inhibitory tyrosine phosphorylation of CDK2 blocked entry into S phase and DNA replication. CDK15A-dependent cell cycle arrest occurs 1 to 2 hours after ultraviolet radiation, whereas the p53-p21 axis affects the cell cycle only several hours after ultraviolet treatment. The researchers thus concluded that the checkpoint response to DNA damage occurs in 2 waves. Overexpression of CDK15A bypassed the mechanism of cell cycle arrest, leading to enhanced DNA damage and decreased cell survival. Mailand et al. (2000) concluded that the results identified specific degradation of CDK15A as part of the DNA damage checkpoint mechanism and suggested how CDK15A overexpression in human cancers might contribute to tumorigenesis.

### CDC7

The CDC7 protein kinase is essential for the G1/S transition and initiation of DNA replication during the cell division cycle in S. cerevisiae. Hsk1 is the S. pombe CDC7 homolog. By searching EST databases for sequences similar to those of CDC7 and Hsk1, Jiang and Hunter, Proc. Nat. Acad. Sci. 94:14320-14325 (1997), which is hereby incorporated by reference in its entirety, identified a partial CDC7 cDNA. They used the partial cDNA to isolate a full-length cDNA from a HeLa cell library. The predicted 574-amino acid human CDC7 protein contains the 11 conserved subdomains found in all protein serine/threonine kinases as well as 3 additional sequences (kinase inserts) between subdomains I and II, VII and VIII, and X and XI. The kinase domains of CDC7 and CDC7 share 44% protein sequence identity. CDC7 has a molecular mass of 64 kD by SDS-PAGE. Using immunofluorescence, the authors demonstrated that CDC7 was predominantly localized in the nucleus. Immune complexes of epitope-tagged CDC7 from mammalian cell lysates phosphorylated histone H1 in vitro. Although the expression levels of CDC7 protein appeared to be constant throughout the cell cycle, the protein kinase activity of CDC7 increased during S phase. Jiang and Hunter (1997) suggested that CDC7 may phosphorylate critical substrate(s) that regulate the G1/S phase transition and/or DNA replication in mammalian cells.

Sato et al., EMBO J. 16:4340-4351 (1997), which is hereby incorporated by reference in its entirety, isolated cDNAs encoding Xenopus and human CDC7 homologs. Northern blot analysis revealed that CDC7 is expressed as 2.4-, 3.5-, and 4.4-kb nmRNAs. The 3.5-kb transcript was detected in all tissues tested, while the 2.4-kb mRNA was testis-specific. Sato et al. (1997) determined that CDC7 phosphorylates the MCM2 and MCM3 proteins in vitro, suggesting that CDC7 may regulate DNA replication by modulating MCM functions. Using Northern blot and dot blot analyses, Hess et al. (Gene 211:133-140 (1998), which is hereby incorporated by reference in its entirety) found that CDC7 was expressed in many normal tissues, but was overexpressed in all transformed cell lines tested and in certain tumor types.

### CDK1

CDK1 is a catalytic subunit of a protein kinase complex, called the M-phase promoting factor, that induces entry into mitosis and is universal among eukaryotes. In the fission yeast Schizosaccharomyces pombe, the gene CDK1 is responsible for controlling the transition from G1 phase to the S phase and from the G2 phase to the M phase of the cell cycle.

Lee et al., (Letter) Nature 333:676-679 (1988), which is hereby incorporated by reference in its entirety, described the regulated expression and phosphorylation of the CDK1 homolog in human and murine in vitro systems. While the yeast cdc2 expression does not appear to be transcriptionally regulated, serum stimulation of human and mouse fibroblasts results in a marked increase in CDK1 transcription. Both the yeast and mammalian systems seem to be regulated by phosphorylation of the CDK1 gene product, a protein kinase of molecular weight 34,000, designated p34(cdc2).

Draetta et al., Nature 336:738-744 (1988), which is hereby incorporated by reference in its entirety, showed that in HeLa cells CDK1 is the most abundant phosphotyrosine-containing protein and its phosphotyrosine content is subject to cell cycle regulation. One site of CDK1 tyrosine phosphorylation in vivo is selectively phosphorylated in vitro by a product of the SRC gene. Liu et al., Molec. Cell. Biol. 17: 571-583 (1997), which is hereby incorporated by reference in its entirety, reported that the kinase MYT1 also phosphorylates CDK1.

Overexpression of the receptor tyrosine kinase ERBB2 confers Taxol resistance in breast cancers. Yu et al., Molec. Cell 2: 581-591 (1998), which is hereby incorporated by reference in its entirety, found that overexpression of ERBB2 inhibits Taxol-induced apoptosis. Taxol activates CDK1 kinase in MDA-MB-435 breast cancer cells, leading to cell cycle arrest at the G2/M phase and, subsequently, apoptosis. A chemical inhibitor of CDK1 and a dominant-negative mutant of CDK1 blocked Taxol-induced apoptosis in these cells. Overexpression of ERBB2 in MDA-MB-435 cells by transfection transcriptionally upregulates CDKN1A which associates with CDK1, inhibits Taxol-mediated CDK1 activation, delays cell entrance to G2/M phase, and thereby inhibits Taxol-induced apoptosis. In CDKN1A antisense-transfected MDA-MB-435 cells or in p21−/− MEF cells, ERBB2 was unable to inhibit Taxol-induced apoptosis. Therefore, CDKN1A participates in the regulation of a G2/M checkpoint that contributes to resistance to Taxol-induced apoptosis in ERBB2-overexpressing breast cancer cells.

ERBB2 overexpression confers resistance to taxol-induced apoptosis by inhibiting p34(CDK1) activation. One mechanism is via ERBB2-mediated upregulation of p21(CIP1), or CDKN1A, which inhibits CDK1. Tan et al., Molec. Cell 9:993-1004 (2002), which is hereby incorporated by reference in its entirety, reported that the inhibitory phosphorylation on tyr15 (Y15) of CDK1 was elevated in ERBB2-overexpressing breast cancer cells and primary tumors. ERBB2 bound to and colocalized with cyclin B-CDK1 complexes and phosphorylated CDK1 Y15. The ERBB2 kinase domain was sufficient to directly phosphorylate CDK1 Y15. Increased CDK1 with phosphorylated Y15 in ERBB2-overexpressing cells corresponded with delayed M phase entry. Expression of a nonphosphorylatable mutant of CDK1 rendered cells more sensitive to taxol-induced apoptosis. Thus, the authors concluded that ERBB2 can confer resistance to taxol-induced apoptosis by directly phosphorylating CDK1.

Konishi et al., Molec. Cell 9:1005-1016 (2002), which is hereby incorporated by reference in its entirety, reported that CDK1 is expressed in postmitotic granule neurons of the developing rat cerebellum and that CDK1 mediates apoptosis of cerebellar granule neurons upon the suppression of neuronal activity. They showed that CDK1 catalyzes the phosphorylation of the BAD protein at a distinct site, ser128, and thereby induces BAD-mediated apoptosis in primary neurons by opposing growth factor inhibition of the apoptotic effect of BAD. Phosphorylation of BAD ser128 was found to inhibit the interaction of growth factor-induced ser136-phosphorylated BAD with 14-3-3 proteins.

### CDK2

The complex formed of CDK1 and cyclin B is required for the G2-to-M transition in cell division. Human cyclin A binds independently to 2 kinases, CDK1 or CDK2. In adenovirus-transformed cells, the viral E1A oncoprotein seems to associate with CDK2/Cyclin A but not with CDK1/cyclin A. Tsai et al., Nature 353: 174-177 (1991), which is hereby incorporated by reference in its entirety, isolated the gene for CDK2, which shares 65% sequence identity with CDK1. They suggested that CDK2 plays a unique role in cell cycle regulation of vertebrate cells.

CDK (e.g., CDK2) activation requires association with cyclins (e.g., CCNE1) and phosphorylation by CAK (CCNH), and leads to cell proliferation. Inhibition of cellular proliferation occurs upon association of CDK inhibitor (e.g., CDKN1B) with a cyclin-CDK complex. Sheaff et al., Genes Dev. 11: 1464-1478 (1997), which is hereby incorporated by reference in its entirety, showed that expression of CCNE1-CDK at physiologic levels of ATP results in phosphorylation of CDKN1B at thr187, leading to elimination of CDKN1B from the cell and progression of the cell cycle from G1 to S phase. At low ATP levels, the inhibitory functions of CDKN1B are enhanced, thereby arresting cell proliferation.

Apoptosis of human endothelial cells after growth factor deprivation is associated with rapid and dramatic upregulation of cyclin A-associated CDK activity. Levkau et al., Molec. Cell 1:553-563 (1998), which is hereby incorporated by reference in its entirety, showed that in apoptotic cells the carboxyl-termini of the CDK inhibitors CDKN1A and CDKN1B are truncated by specific cleavage. The enzyme involved in this cleavage is CASP3 and/or a CASP3-like caspase. After cleavage, CDKN1A loses its nuclear localization sequence and exits the nucleus. Cleavage of CDKN1A and CDKN1B resulted in a substantial reduction in their association with nuclear cyclin-CDK complexes, leading to a dramatic induction of CDK2 activity. Dominant-negative CDK2, as well as a mutant CDKN1A resistant to caspase cleavage, partially suppressed apoptosis. These data suggested that CDK2 activation, through caspase-mediated cleavage of CDK inhibitors, may be instrumental in the execution of apoptosis following caspase activation.

Hinchcliffe et al., Science 283:851-854 (1999), which is hereby incorporated by reference in its entirety, developed a Xenopus egg extract arrested in S phase that supported repeated assembly of daughter centrosomes. Multiple rounds of centrosome reproduction were blocked by selective inactivation of CDK2-Cyclin E and were restored by addition of purified CDK-cyclin E. Confocal microscopy revealed that cyclin E was localized at the centrosome. The authors concluded that CDK2-Cyclin E activity is required for centrosome duplication during S phase and that these results suggested a mechanism that could coordinate centrosome reproduction with cycles of DNA synthesis and mitosis.

Inhibition of CDK2, a positive regulator of eukaryotic cell cycle progression, may represent a therapeutic strategy for prevention of chemotherapy-induced alopecia by arresting the cell cycle and reducing the sensitivity of the epithelium to many cell cycle-active antitumor agents. Davis et al., Science 291:134-137 (2001), which is hereby incorporated by reference in its entirety, developed potent small-molecule inhibitors of CDK2 using structure-based methods. Topical application of these compounds in a neonatal rat model of chemotherapy-induced alopecia reduced hair loss at the site of application in 33 to 50% of the animals. Thus, Davis et al. (2001) concluded that inhibition of CDK2 represents a potentially useful approach for the prevention of chemotherapy-induced alopecia in cancer patients.

Falck et al., Nature Genet. 30:290-294 (2002), which is hereby incorporated by reference in its entirety, demonstrated that experimental blockade of either the NBS1-MRE11 function or the CHK2-triggered events leads to a partial radioresistant DNA synthesis phenotype in human cells. In contrast, concomitant interference with NBS1-MRE11 and the CHK2-CDC25A-CDK2 pathways entirely abolishes inhibition of DNA synthesis induced by ionizing radiation, resulting in complete RDS analogous to that caused by defective ATM. In addition, CDK2-dependent loading of CDC45 onto replication origins, a prerequisite for recruitment of DNA polymerase, was prevented upon irradiation of normal or NBS1/MRE11-defective cells but not cells with defective ATM. Falck et al. (2002) concluded that in response to ionizing radiation, phosphorylation of NBS 1 and CHK2 by ATM triggers 2 parallel branches of the DNA damage-dependent S-phase checkpoint that cooperate by inhibiting distinct steps of DNA replication.

### CDK8

Cyclins are positive regulatory subunits of cyclin-dependent kinases (CDKs). In S. cerevisiae, the CDK SRB10 has been shown to interact with SRB11, a cyclin related to mammalian cyclin C. The SRB10-SRB11 complex is part of the RNA polymerase II holoenzyme and acts as a regulator of transcription. To identify human protein kinases with a role in cell cycle control, Schultz and Nigg, Cell Growth Differ. 4:821-830 (1993), which is hereby incorporated by reference in its entirety, performed PCR with degenerate oligonucleotides based on conserved motifs in the catalytic domain of the Aspergillus nidulans NIMA protein kinase. They isolated 41 distinct promyelocytic leukemia cell line cDNAs, including 1 partial cDNA designated K35. Tassan et al., Proc. Nat. Acad. Sci. 92:8871-8875 (1995), which is hereby incorporated by reference in its entirety, noted that K35 appears to be structurally related to CDKs. By screening a human testis cDNA library with K35, they isolated cDNAs corresponding to the entire coding region of CDK8. The predicted 464-amino acid protein contains the sequence motifs and 11 subdomains characteristic of a serine/threonine-specific kinase. The protein sequences of CDK8 and SRB10 are 48% identical over subdomains III to XI, and the 2 proteins have several common features. CDK8 migrates as a 53-kD protein on Western blots of HeLa cell extracts. Coimmunoprecipitation experiments demonstrated that CDK8 interacted with cyclin C both in vitro and in vivo. Tassan et al. (1995) proposed that CDK8-cyclin C might be functionally associated with the mammalian transcription apparatus.

Mammalian CDK8 and cyclin C are components of the RNA polymerase II holoenzyme complex, where they function as a protein kinase that phosphorylates the C-terminal domain of the largest subunit of RNA polymerase II. The CDK8/cyclin C protein complex is also found in a number of mammalian ‘Mediator’-like protein complexes, which repress activated transcription independently of the C-terminal domain in vitro. Akoulitchev et al., Nature 407:102-106 (2000), which is hereby incorporated by reference in its entirety, demonstrated that CDK8/cyclin C can regulate transcription by targeting the CDK7/Cyclin H subunits of the general transcription initiation factor IIH. CDK8 phosphorylates mammalian cyclin H at serine 5 and serine 304 both in vitro and in vivo, in the vicinity of its functionally unique N— and C-terminal alpha-helical domains. This phosphorylation represses both the ability of TFIIH to activate transcription and its C-terminal kinase activity. In addition, mimicking CDK8 phosphorylation of cyclin H in vivo has a dominant-negative effect on cell growth. Akoulitchev et al. (2000) concluded that their results linked the Mediator complex and the basal transcription machinery by a regulatory pathway involving 2 cyclin-dependent kinases. This pathway appears to be unique to higher organisms.

### CKS2

The Cks1 protein is a component of the Cdc28 protein kinase in the budding yeast Saccharomyces cerevisiae. Richardson et al., Genes Dev. 4:1332-1344 (1990), which is hereby incorporated by reference in its entirety, cloned 2 human homologs of the Cks1 gene of yeast. Designated CKS1 and CKS2, both encode proteins of 79 amino acids that share considerable homology at the amino acid level with the products of the corresponding gene in S. cerevisiae and another gene in the fission yeast Schizosaccharomyces pombe. Both human homologs were capable of rescuing a null mutation of the S. cerevisiae Cks1 gene when expressed from the S. cerevisiae GAL1 promoter. Linked to Sepharose beads, the CKS1 and CKS2 proteins could bind the CDC28/CDC2 protein kinase from both S. cerevisiae and human cells. The CKS1 and CKS2 mRNAs are expressed in different patterns through the cell cycle in HeLa cells, which reflects specialized roles for the encoded proteins.

Spruck et al., Science 300: 647-650 (2003), which is hereby incorporated by reference in its entirety, generated mice lacking CKS2 and found them to be viable but sterile in both sexes. Sterility is due to failure of both male and female germ cells to progress past the first meiotic metaphase. The chromosomal events through the end of prophase I are normal in both Cks2-null males and females, suggesting that the phenotype is due directly to failure to enter anaphase and not a consequence of a checkpoint-mediated metaphase I arrest.

### CKS1

The Cks1 protein is a component of the Cdc28 protein kinase in the budding yeast Saccharomyces cerevisiae. Richardson et al., Genes Dev. 4:1332-1344 (1990), which is hereby incorporated by reference in its entirety, cloned 2 human homologs of the Cks1 gene of yeast. Designated CKS 1 and CKS2, both encode proteins of 79 amino acids that share considerable homology at the amino acid level with the products of the corresponding gene in S. cerevisiae and another gene in the fission yeast Schizosaccharomyces pombe. The CKS1 and CKS2 mRNAs were found to be expressed in different patterns through the cell cycle in HeLa cells, which reflects specialized roles for the encoded proteins.

Richardson et al. (1990) showed that both human Cks1 homologs were capable of rescuing a null mutation of the S. cerevisiae Cks1 gene when expressed from the S. cerevisiae GAL1 promoter. Linked to Sepharose beads, the CKS1 and CKS2 proteins could bind the CDC28/CDC2 protein kinase from both S. cerevisiae and human cells.

By investigating the essential role of CKS1 in S. cerevisiae, Morris et al., Nature 423:1009-1013 (2003), which is hereby incorporated by reference in its entirety, demonstrated that the protein is primarily involved in promoting mitosis by modulating the transcriptional activation of CDC20. CKS1 is required for both the periodic dissociation of CDC28 kinase from the CDC20 promoter and the periodic association of the proteasome with the promoter. Morris et al. (2003) proposed that the essential role of CKS1 is to recruit the proteasome to, and/or dissociate the CDC28 kinase from, the CDC20 promoter, thus facilitating transcription by remodeling transcriptional complexes or chromatin associated with the CDC20 gene.

Bourne et al., Cell 84:863-874 (1996), which is hereby incorporated by reference in its entirety, analyzed the crystal structure of the CDK-CKS1 complex and defined the critical protein domains involved in the interaction of the 2 molecules. They tested the biologic importance of the structure-based model by constructing mutant alleles of CKS1 that led to decreased interaction with CDK2. Bourne et al. (1996) concluded that the structural analysis revealed the mode of CDK binding to CKS1, suggested a possible mechanism of cooperativity and self regulation of CKS proteins during the cell cycle, and implicated CKS as a targeting or matchmaking protein for CDK and at least 1 other phosphoprotein.

### CUL1

Kipreos et al., Cell 85:829-839 (1996), which is hereby incorporated by reference in its entirety, found that mutations in the cullin-1 (cul1) gene of C. elegans cause hyperplasia of all tissues. They determined that cull is a negative regulator of the cell cycle; in cull mutants, the G1-to-S-phase progression is accelerated, overriding mechanisms for mitotic arrest and producing abnormally small cells. Searches of EST databases revealed that cull is a member of a conserved gene family, with at least 5 members in nematodes, 6 in humans, and 3 in S. cerevisiae. Human CUL1 is an ortholog of nematode cul1.

Michel and Xiong, Cell Growth Differ. 9:435-449 (1998), which is hereby incorporated by reference in its entirety, stated that CUL1 has homology to yeast Cdc53, which is part of a complex known as SCF that mediates the ubiquitin-dependent degradation of G1 cycles and cyclin-dependent kinase inhibitors. SCF complexes are composed of SKP1, Cdc53, and an F box-containing protein, which may confer substrate specificity. These authors found that interaction of the predicted 776-amino acid human CUL1 protein with SKP1 is mediated through the N-terminal domains of both proteins. Immunoprecipitation studies and Western blot analysis revealed that the steady-state levels of both CUL1 and SKP1, as well as their association with one another, remain relatively constant throughout the cell cycle and in postmitotic cells. However, none of the other human cullins tested interacted with SKP1. Michel and Xiong (1998) determined that via SKP1, CUL1 forms a complex with SKP2, an F box-containing protein, and cyclin A. The authors concluded that the SCF proteolytic pathway is evolutionarily conserved and is used by mammalian CUL1, while the other cullin proteins may use a SKP1/F-box-independent pathway to mediate protein degradation.

Maniatis, Genes Dev. 13: 505-510 (1999), which is hereby incorporated by reference in its entirety, reviewed the work of Winston et al., Genes Dev. 13:270-283 (1999), which is hereby incorporated by reference in its entirety, and others concerning the SCF ubiquitin ligase complex. CUL1 acts as a scaffold for SKP1 and the F-box-containing BTRC protein in the SCF complex, which regulates the function of nuclear factor kappa-B and beta-catenin.

Yu et al., Proc. Nat. Acad. Sci. 95:11324-11329 (1998), which is hereby incorporated by reference in its entirety, reported studies suggesting that the p19 (SKP1)/p45 (SKP2)/CUL1 complex is likely to function as a conserved ubiquitin E3 enzyme that regulates the mammalian G1/S transition by specifically targeting mammalian G1 cell cycle regulators, such as p21 and cyclin D proteins, for ubiquitin-dependent degradation.

The sequential timing of cell cycle transitions is primarily governed by the availability and activity of key cell cycle proteins. Studies in yeast identified a class of ubiquitin ligases (E3 enzymes) called SCF complexes, which regulate the abundance of proteins that promote and inhibit cell cycle progression at the transition between G1 and S phases. SCF complexes consist of 3 invariable components, SKP1, CUL1 (CDC53 in yeast), and RBX1, and a variable F-box protein that recruits a specific cellular protein to the ubiquitin pathway for degradation. To study the role of CUL1 in mammalian development and cell cycle regulation, Dealy et al., Nature Genet. 23:245-248 (1999), which is hereby incorporated by reference in its entirety, generated mice deficient for Cul1 and analyzed null embryos and heterozygous cell lines. They showed that Cul1 is required for early mouse development and that Cul1 mutants fail to regulate the abundance of the G1 cyclin, cyclin E1, during embryogenesis.

Zheng et al., Molec. Cell 10:1519-1526 (2002), which is hereby incorporated by reference in its entirety, determined that the majority of CUL1 is in a complex with CAND1 and ROC1 independent of SKP1 and the F box protein SKP2. Both in vivo and in vitro, CAND1 prevented binding of SKP1 and SKP2 to CUL1, while dissociation of CAND1 from CUL1 promoted the reverse reaction. Neddylation of CUL1 or the presence of SKP1 and ATP caused CAND1 dissociation. These data suggested that CAND1 regulates the formation of the SCF complex and that its dissociation from CUL1 is coupled with the incorporation of F box proteins into the SCF complex, causing their destabilization.

Liu et al., Molec. Cell 10:1511-1518 (2002), which is hereby incorporated by reference in its entirety, showed that CAND1 selectively binds to unneddylated CUL1 and is dissociated by CUL1 neddylation. CAND1 formed a ternary complex with CUL1 and ROC1. It dissociated SKP1 from CUL1 and inhibited SCF ligase activity in vitro. Suppression of CAND1 in vivo increased the level of the CUL1-SKP1 complex. The authors concluded that, by restricting SKP1-CUL1 interaction, CAND1 regulates the assembly of productive SCF ubiquitin ligases, allowing a common CUL1-ROC core to be utilized by a large number of SKP1-F box-substrate subcomplexes.

Staropoli et al., Neuron 37:735-749 (2003), which is hereby incorporated by reference in its entirety, demonstrated that parkin associates with the F-box proteins FBXW7 and CUL1 in a distinct ubiquitin ligase complex. FBXW7 serves to target the ligase activity to cyclin E, a protein previously implicated in the regulation of neuronal apoptosis. In cells transfected with the parkin T240R mutation, parkin deficiency potentiated the accumulation of cyclin E in cultured postmitotic neurons exposed to the glutamatergic excitotoxin kainate and promoted their apoptosis. Furthermore, parkin overexpression attenuated cyclin E accumulation in toxin-treated neurons and protected them from apoptosis.

### CUL2

Kipreos et al., Cell 85:829-839 (1996), which is hereby incorporated by reference in its entirety, identified a conserved gene family, designated cullins, with at least 5 members in nematodes, 6 in humans, and 3 in S. cerevisiae. Human CUL2 is an ortholog of nematode cul2. Michel and Xiong, Cell Growth Differ. 9: 435-449 (1998), which is hereby incorporated by reference in its entirety, identified human CUL2 cDNAs and reported that the predicted protein is 745 amino acids long.

Pause et al., Proc. Nat. Acad. Sci. 94:2156-2161 (1997), which is hereby incorporated by reference in its entirety, reported that the protein sequences of human and C. elegans cul2 are 45% identical. Using immunofluorescence, they showed that CUL2 is a cytosolic protein that can be translocated to the nucleus by VHL. Both Pause et al. (1997) and Lonergan et al., Molec. Cell. Biol. 18: 732-741 (1998), which is hereby incorporated by reference in its entirety, found that CUL2 specifically associates with the trimeric VHL-elongin B-elongin C, or VBC, complex in vitro and in vivo. This association was disrupted by mutations in VHL that disrupt elongin binding. Nearly 70% of the naturally-occurring cancer-disposing mutations in VHL abrogate elongin binding, suggesting that binding to elongin-CUL2 complexes contributes to the ability of VHL to suppress tumor growth in vivo. Pause et al. (1997) suggested that CUL2 is a candidate tumor suppressor gene, as has been proposed for CUL1. Lonergan et al. (1998) demonstrated that formation of the VBC-CUL2 complexes is linked to the regulation of hypoxia-inducible mRNAs by VHL. They proposed a model for this regulation based on the similarity of elongin C and CUL2 to SKP1 and CUL1, which have been shown in yeast to form complexes that target specific proteins for ubiquitin-dependent proteolysis.

### CUL3

Kipreos et al., Cell 85:829-839 (1996), which is hereby incorporated by reference in its entirety, identified a conserved gene family, designated cullins, with at least 5 members in nematodes, 6 in humans, and 3 in S. cerevisiae. Human CUL3 is an ortholog of nematode cul3. Michel and Xiong, Cell Growth Differ. 9:435-449 (1998), which is hereby incorporated by reference in its entirety, identified human CUL3 cDNAs and reported that the predicted protein is 768 amino acids long. Ishikawa et al., DNA Res. 5:169-176 (1998), which is hereby incorporated by reference in its entirety, isolated a CUL3 cDNA, KIAA0617, as 1 of 100 brain cDNAs encoding large proteins. Using RT-PCR, they found that CUL3 is expressed in several tissues. Du et al., J. Biol. Chem. 273:24289-24292 (1998), which is hereby incorporated by reference in its entirety, identified CUL3 as a gene whose expression in human fibroblasts is induced by phorbol 12-myristate 13-acetate (PMA) and suppressed by salicylate. They reported that the sequences of the human and C. elegans cul3 proteins share 46% identity. Northern blot analysis revealed that CUL3 is expressed as major 2.8- and minor 4.3-kb transcripts in various human tissues, with the highest levels in skeletal muscle and heart.

### E2F-3

The E2F family of transcription factors activate genes that control DNA synthesis (Chellappan et al., Cell 65:1053 (1991), which is hereby incorporated by reference in its entirety). Cyclin E2 is rate limiting for G1 progression and its expression is regulated by E2F. E2F is a pivotal role in coordination of events connected with proliferation, cell cycle arrest, and apoptosis. E2F transcription factors also regulate cyclin A gene expression. Cyclins E and A are known to be active in G1 phase, which is the interval that cells respond to extracellular stimuli. G1 regulators are important in accelerating or braking the cell cycle (Sherr, Cancer Res. 60:3689 (2000), which is hereby incorporated by reference in its entirety). Because increased BVR expression has been shown to upregulate cyclins A, E1 and E2, as well as the transcription factor E2F-3, it is believed that BVR can be used to control the cell division cycle and alter periods associated with DNA replication, thus allowing for DNA repair and cell differentiation.

MYC induces transcription of the E2F1, E2F2, and E2F3 genes. Using primary mouse embryo fibroblasts deleted for individual E2f genes, Leone et al., Molec. Cell 8:105-113 (2001), which is hereby incorporated by reference in its entirety, showed that MYC-induced S phase and apoptosis requires distinct E2F activities. The ability of Myc to induce S phase was impaired in the absence of either E2f2 or E2f3 but not E2f1 or E2f4. In contrast, the ability of Myc to induce apoptosis was markedly reduced in cells deleted for E2f1 but not E2f2 or E2f3. The authors proposed that the induction of specific E2F activities is an essential component in the MYC pathways that control cell proliferation and cell fate decisions.

The retinoblastoma tumor suppressor (Rb) pathway is believed to have a critical role in the control of cellular proliferation by regulating E2F activities. E2F1, E2F2, and E2F3 belong to a subclass of E2F factors thought to act as transcriptional activators important for progression through the G1/S transition. Wu et al., Nature 414: 457-462 (2001), which is hereby incorporated by reference in its entirety, used a conditional gene targeting approach to demonstrate that combined loss of these 3 E2F factors severely affects E2F target expression and completely abolishes the ability of mouse embryonic fibroblasts to enter S phase, progress through mitosis, and proliferate. Loss of E2F function results in elevation of CIP1 protein, leading to a decrease in cyclin-dependent kinase activity and Rb phosphorylation. Wu et al. (2001) concluded that these findings suggest a function for this subclass of E2F transcriptional activators in a positive feedback loop, through downmodulation of CIP1, that leads to the inactivation of Rb-dependent repression and S phase entry.

By targeting the entire subclass of E2F transcriptional activators, Wu et al. (2001) provided direct genetic evidence for their essential role in cell cycle progression, proliferation, and development. Wu et al. (2001) initially generated and interbred E2f1, E2f2, and E2f3 mutant mice, and found that although mice null for E2f1 and E2f2 were viable and developed to adulthood, mice null for E2f1 and E2f3 or E2f2 and E2f3 died early during embryonic development, at or just before embryonic day 9.5, pointing to a central role for E2f3 in mouse development.

Cloud et al., Molec. Cell. Biol. 22:2663-2672 (2002), which is hereby incorporated by reference in its entirety, generated E2f3-null mice. They found that E2f3 was essential for embryonic viability in the pure 129/Sv background, but that the presence of C57BL/6 alleles yielded some adult survivors. Although growth retarded, surviving E2f3−/− animals were initially healthy and exhibited no obvious tumor phenotype. They died prematurely, however, with signs typical of congestive heart failure, a defect completely distinct from those reported in E2f1-null mice. Cloud et al. (2002) also generated E2f1/E2f3 compound mutant mice and found that almost all of the developmental and age-related defects arising in the individual E2f1- or E2f3-null mice were exacerbated by the mutation of the other E2f.

### MAD2L1

Li and Benezra, Science 274:246-248 (1996), which is hereby incorporated by reference in its entirety, reviewed mitotic checkpoint control mechanisms and noted that these mechanisms check the cells preparedness to undergo division. Through these mechanisms cell cycle progression is blocked before the irreversible events associated with anaphase if either the mitotic spindle apparatus is not properly assembled or the kinetochore is not properly attached to the spindle. Mitotic arrest-deficient-2 (MAD2) is one of 6 yeast genes that are required for execution of the mitotic checkpoint. Dysfunction of MAD2 may lead to malignancy or degeneration of cells (Li and Nicklas, Nature 373: 630-632 (1995); Li and Benezra, Science 274: 246-248 (1996), which are hereby incorporated by reference in their entirety).

Li and Benezra (1996) isolated a human homolog of MAD2 (MAD2L1) in a screen for high copy-number suppressors of thiabendazole sensitivity in yeast lacking CBF1, a component of the kinetochore. (Thiabendazole is a mitotic spindle assembly inhibitor.) The gene encodes a 205-amino acid polypeptide. DNA sequence determination revealed that the open reading frame of the human clone is 60% identical to the yeast MAD2 gene. They used antibody electroporation experiments to demonstrate that the human MAD2 gene was a necessary component of the mitotic checkpoint in HeLa cells. Through immunofluorescence studies they demonstrated that the human MAD2 protein is localized at the kinetochore after chromosome condensation but that it is no longer observed at the kinetochore in metaphase. Based on this observation they proposed that MAD2 monitors the completeness of the spindle kinetochore attachment. Li and Benezra (1996) demonstrated that a human breast tumor cell line T47D has reduced MAD2 expression and that it failed to arrest in mitosis after nocodazole treatment. They proposed that loss of MAD2 function might also lead to aberrant chromosome segregation in mammalian cells.

Chen et al., Science 274:242-245 (1996), which is hereby incorporated by reference in its entirety, isolated a Xenopus homolog of yeast MAD2. They reported that the product of this gene plays an essential role in spindle checkpoint assembly. The protein associated with unattached kinetochores in prometaphase and nocodazole treated cells and disappeared from kinetochores at metaphase.

Luo et al., Molec. Cell 9:59-71 (2002), which is hereby incorporated by reference in its entirety, showed that RNA interference-mediated suppression of MAD1 function in mammalian cells caused loss of MAD2 kinetochore localization and impairment of the spindle checkpoint. MAD1 and CDC20 contain MAD2-binding motifs that share a common consensus, and the authors identified a class of MAD2-binding peptides (MBPs) with a similar consensus. Binding of one of these ligands, MBP1, triggered an extensive rearrangement of the tertiary structure of MAD2. MAD2 also underwent a similar striking structural change upon binding to a MAD1 or CDC20 binding motif peptide. These data suggested that, upon checkpoint activation, MAD1 recruits MAD2 to unattached kinetochores and may promote binding of MAD2 to CDC20.

The initiation of chromosome segregation at anaphase is linked by the spindle assembly checkpoint to the completion of chromosome-microtubule attachment during metaphase. To determine the function of the Mad2 protein during normal cell division, Dobles et al., Cell 101:635-645 (2000), which is hereby incorporated by reference in its entirety, knocked out the Mad2 gene in mice. They found that embryonic cells lacking Mad2 at embryonic day 5.5, like mad2 yeast, grew normally but were unable to arrest in response to spindle disruption. At embryonic day 6.5, the cells of the epiblast began rapid cell division, and the absence of a checkpoint resulted in widespread chromosome missegregation and apoptosis. In contrast, the postmitotic trophoblast giant cells survived without Mad2. Thus, the spindle assembly checkpoint is required for accurate chromosome segregation in mitotic mouse cells and for embryonic viability, even in the absence of spindle damage.

Shonn et al., Science 289: 300-303 (2000), which is hereby incorporated by reference in its entirety, characterized the spindle checkpoint in meiosis of S. cerevisiae by comparing wildtype and mad2-deficient yeast. In the absence of the checkpoint, the frequency of meiosis I missegregation increased with increasing chromosome length, reaching 19% for the longest chromosome. Meiosis I nondisjunction in spindle checkpoint mutants could be prevented by delaying the onset of anaphase. In a recombinant-defective mutant, the checkpoint delayed the biochemical events of anaphase I, suggesting that chromosomes that are attached to microtubules but are not under tension can activate the spindle checkpoint. Spindle checkpoint mutants reduced the accuracy of chromosome segregation in meiosis I much more than that in meiosis II, suggesting that checkpoint defects may contribute to Down syndrome. Shonn et al. (2000) showed that the budding yeast spindle checkpoint, which is largely dispensable in wildtype mitosis, plays a critical role in meiotic chromosome segregation. They suggested that the difference may reflect the different chromosome linkages in mitosis and meiosis I. In mitosis, sister chromatid cohesion forces sister kinetochores to face opposite spindle poles. In meiosis I, homologs are linked at sites of recombination that can be far from the kinetochores, creating a floppy linkage. If the nearest recombination event is further from the centromere on long chromosomes, this idea may explain why long chromosomes preferentially nondisjoin in checkpoint-defective cells.

Michel et al., Nature 409:355-359 (2001), which is hereby incorporated by reference in its entirety, reported that deletion of one MAD2 allele results in a defective mitotic checkpoint in both human cancer cells and murine primary embryonic fibroblasts. Checkpoint-defective cells show premature sister chromatid separation in the presence of spindle inhibitors and an elevated rate of chromosome missegregation events in the absence of these agents. Furthermore, Mad2± mice develop lung tumors at high rates after long latencies, implicating defects in the mitotic checkpoint in tumorigenesis.

### MCM6

The MCM genes were originally identified in yeast defective in minichromosome maintenance and have since been shown to play roles in the progression of the cell cycle; many are cell division control genes. MCMs 2 through 7 are thought to be ‘DNA licensing factors’ which bind to the DNA after mitosis and enable DNA replication before being removed during S phase. Harvey et al., FEBS Lett. 398: 135-140 (1996), which is hereby incorporated by reference in its entirety, identified the human MCM6 gene and mapped it to 2q21 by fluorescence in situ hybridization. MCM6 is expressed in a wide variety of human adult and fetal tissues.

### RBX1

The VHL protein is part of a complex that includes elongin B, elongin C, and cullin-2(CUL2), proteins associated with transcriptional elongation and ubiquitination. Components of the VCB (VHL-elongin C/elongin B) complex share sequence similarities with the E3 ubiquitin ligase complexes, SCF (SKP1)-CUL1-F-box protein) and APC (anaphase promoting complex). F-box proteins, such as S. cerevisiae Cdc4 and Grr1, are adaptor proteins that recruit different binding partners to SCF (Tyers and Willems, Science 284: 602-604 (1999), which is hereby incorporated by reference in its entirety).

Kamura et al., Science 284:657-661 (1999), which is hereby incorporated by reference in its entirety, purified the endogenous VHL complex from rat liver and determined the partial protein sequence of a 16-kD protein component. By searching an EST database with the peptide sequences, these authors identified human and mouse cDNAs encoding a predicted 108-amino acid protein. They designated the protein RBX1 (RING-box protein-1) because it contained a RING-H2 finger-like motif. The mouse and human RBX1 proteins are identical, and there are RBX1 homologs in Drosophila, C. elegans, and S. cerevisiae. Kamura et al. (1999) demonstrated that RBX1 interacts with both CUL1 and CUL2. They found that yeast Rbx1 is a subunit and a potent activator of the SCF-Cdc4 complex that is required for ubiquitination of the cyclin-dependent kinase inhibitor Sic1 and for the G1-to-S cell cycle transition. Mammalian RBX1 rescued the viability defect in yeast rbx1 mutants. The authors concluded that the presence of RBX1 as a component of both the VHL and SCF-Cdc4 complexes extends the structural similarity between these 2 complexes and raises the possibility that the VHL complex may function as a ubiquitin ligase for target proteins. Skowyra et al., Science 284: 662-665 (1999), which is hereby incorporated by reference in its entirety, found that Rbx1 is part of the yeast SCF-Grr1 complex, which ubiquitinates the phosphorylated G1 cyclin cln1.

Using mouse cullin-4A as bait in a yeast 2-hybrid screen of a human HeLa pGAD cDNA library, Ohta et al., Molec. Cell 3:535-541 (1999), which is hereby incorporated by reference in its entirety, identified 2 highly conserved RING finger proteins, which they referred to as ROC1 and ROC2 (RBX1 and RBX2), which are homologous to APC11, a subunit of the anaphase-promoting complex. The RBX1 and RBX2 proteins commonly interact with all cullins. Yeast RBX1 encodes an essential gene whose reduced expression resulted in multiple, elongated buds and accumulation of Sic1 and Cln2 proteins. RBX1 and APC11 immunocomplexes can catalyze isopeptide ligations to form polyubiquitin chains in an E1- and E2-dependent manner. RBX1 mutations completely abolished their ligase activity without noticeable changes in associated proteins. Ubiquitination of phosphorylated I-kappa-B-alpha can be catalyzed by the RBX1 immunocomplex in vitro. Hence, combinations of RBX/APC11 and cullin proteins potentially constitute a wide variety of ubiquitin ligases.

### RAD50

The S. cerevisiae Rad50 gene encodes a protein that is essential for double-stranded DNA break repair by nonhomologous DNA end joining and chromosomal integration. The yeast Rad50, Mre11, and Xrs2 proteins appear to act in a multiprotein complex, consistent with the observation that mutations in these genes confer nearly identical phenotypes of no meiotic recombination and elevated rates of homologous mitotic recombination. By direct selection of cDNAs from the 5q23-q31 chromosomal interval, Dolganov et al., Molec. Cell Biol. 16:4832-4841 (1996), which is hereby incorporated by reference in its entirety, isolated a cDNA encoding a human Rad50 homolog. The human RAD50 gene spans 100 to 130 kb. Northern blot analysis revealed that the RAD50 gene was expressed as a 5.5-kb mRNA predominantly in testis. A faint 7-kb transcript, which the authors considered to be an mRNA with an alternatively processed 3-prime end, was also detected. Yeast Rad50 and the predicted 1,312-amino acid human RAD50 protein share more than 50% identity in their N— and C-termini. The central heptad repeat domains of the proteins have relatively divergent primary sequences but are predicted to adopt very similar coiled-coil structures. Using immunoprecipitation, Dolganov et al. (1996) demonstrated that the 153-kD RAD50 is stably associated with MRE11 in a protein complex, which may also include proteins of 95 kD, 200 kD, and 350 kD.

By inclusion within mapped clones and by analysis of somatic cell hybrids, Dolganov et al. (1996) mapped the RAD50 gene to 5q31. They suggested that a recombinational DNA repair deficiency may be associated with the development of myeloid leukemia, since this chromosomal region is frequently altered in acute myeloid leukemia and myelodysplastic disease.

Trujillo et al., J. Biol. Chem. 273:21447-21450 (1998), which is hereby incorporated by reference in its entirety, determined that the 95-kD protein in the mammalian cell nuclear complex containing RAD50 and MRE11 is nibrin, or p95, the protein encoded by the gene mutated in Nijmegen breakage syndrome (NBS). The RAD50 complex possessed manganese-dependent single-stranded DNA endonuclease and 3-prime to 5-prime exonuclease activities. The authors stated that these nuclease activities are likely to be important for recombination, repair, and genomic stability. Carney et al., Cell 93:477-486 (1998), which is hereby incorporated by reference in its entirety, demonstrated that p95 is an integral member of the MRE11/RAD50 complex and that the function of this complex is impaired in cells from NBS patients. They stated that although p95 has little sequence homology to yeast Xrs2, the 2 proteins can be considered functional analogs since they link the conserved activities of MRE11/RAD50 to the cellular DNA damage response in their respective organisms.

Zhong et al., Science 285:747-750 (1999), which is hereby incorporated by reference in its entirety, showed that BRCA1 interacts in vitro and in vivo with RAD50. Formation of irradiation-induced foci positive for BRCA1, RAD50, MRE11, or p95 was dramatically reduced in HCC/1937 breast cancer cells carrying a homozygous mutation in BRCA1 but was restored by transfection of wildtype BRCA1. Ectopic expression of wildtype, but not mutated, BRCA1 in these cells rendered them less sensitive to the DNA damage agent methyl methanesulfonate. These data suggested to the authors that BRCA1 is important for the cellular responses to DNA damage that are mediated by the RAD50-MRE11-p95 complex.

Wang et al., Genes Dev. 14:927-939 (2000), which is hereby incorporated by reference in its entirety, used immunoprecipitation and mass spectrometry analyses to identify BRCA1-associated proteins. They found that BRCA1 is part of a large multisubunit protein complex of tumor suppressors, DNA damage sensors, and signal transducers. They named this complex BASC, for ‘BRCA1-associated genome surveillance complex.’ Among the DNA repair proteins identified in the complex were ATM, BLM, MSH2, MSH6, MLH1, the RAD50-MRE11-NBS1 complex, and the RFC1-RFC2-RFC4 complex. Confocal microscopy demonstrated that BRCA1, BLM, and the RAD50-MRE11-NBS1 complex colocalize to large nuclear foci. Wang et al. (2000) suggested that BASC may serve as a sensor of abnormal DNA structures and/or as a regulator of the postreplication repair process.

Telomeres allow cells to distinguish natural chromosome ends from damaged DNA and protect the ends from degradation and fusion. In human cells, telomere protection depends on the TTAGGG repeat-binding factor, TRF2, which may remodel telomeres into large duplex loops (t-loops). Zhu et al., Nature Genet. 25:347-352 (2000), which is hereby incorporated by reference in its entirety, showed by nanoelectrospray tandem mass spectrometry that RAD50 protein is present in TRF2 immunocomplexes. Coimmunoprecipitation studies showed that a small fraction of RAD50, MRE11, and p95 is associated with TRF2. Indirect immunofluorescence demonstrated the presence of RAD50 and MRE11 at interphase telomeres. NBS1 was associated with TRF2 and telomeres in S phase, but not in G1 or G2. Although the MRE11 complex accumulated in irradiation-induced foci (IRIFs) in response to gamma-irradiation, TRF2 did not relocate to IRIFs and irradiation did not affect the association of TRF2 with the MRE11 complex, arguing against a role for TRF2 in double-strand break repair. Zhu et al. (2000) proposed that the MRE11 complex functions at telomeres, possibly by modulating t-loop formation.

The MRE11/RAD50 protein complex functions in diverse aspects of the cellular response to double strand breaks (DSBs), including the detection of DNA damage, the activation of cell cycle checkpoints, and DSB repair. Whereas genetic analyses in S. cerevisiae have provided insight regarding DSB repair functions of this highly conserved complex, the implication of the human complex in Nijmegen breakage syndrome reveals its role in cell cycle checkpoint functions. Luo et al., Proc. Nat. Acad. Sci. 96:7376-7381 (1999), which is hereby incorporated by reference in its entirety, established mice with mutation in the mouse Rad50 gene and examined the role of the Mre11/Rad50 protein complex in the DNA damage response. Early embryonic cells deficient in Rad50 were hypersensitive to ionizing radiation, consistent with a role for this complex in the repair of ionizing radiation-induced DSBs. However, the null Rad50 mutation was lethal in cultured embryonic stem cells and in early developing embryos, indicating that the mammalian protein complex mediates functions in normally growing cells that are essential for viability.

In mammalian cells, a conserved multiprotein complex of MRE11, RAD50, and NBS1 is important for double-strand break repair, meiotic recombination, and telomere maintenance. In the absence of the early region E4, the double-stranded genome of adenoviruses is joined into concatamers too large to be packaged. Stracker et al., Nature 418:348-352 (2002), which is hereby incorporated by reference in its entirety, investigated the cellular proteins involved in the concatamer formation and how they are inactivated by E4 products during a wildtype infection. They demonstrated that concatamerization requires functional MRE11 and NBS1, and that these proteins are found at foci adjacent to viral replication centers. Infection with wildtype virus results in both reorganization and degradation of members of the MRE11-RAD50-NBS1 complex. These activities are mediated by 3 viral oncoproteins that prevent concatamerization. This targeting of cellular proteins involved in the genomic stability suggested a mechanism for ‘hit-and-run’ transformation observed for these viral oncoproteins.

Hopfaer et al., Nature 418: 562-566 (2002), which is hereby incorporated by reference in its entirety, presented a 2.2-angstrom crystal structure of the Rad50 coiled-coil region that revealed an unexpected dimer interface at the apex of the coiled coils in which pairs of conserved cys-x-x-cys motifs form interlocking hooks that bind one zinc ion. Biochemical, x-ray, and electron microscopy data indicated that these hooks can join oppositely protruding Rad5O coiled-coil domains to form a flexible bridge of up to 1,200 angstroms. This suggested a function for the long insertion in the Rad50 ABC-ATPase. The Rad50 hook is functional, since mutations in this motif confer radiation sensitivity in yeast and disrupt binding at the distant Mre11 nuclease interface. Hopfner et al. (2002) concluded that their data support an architectural role for the Rad50 coiled coils in forming metal-mediated bridging complexes between 2 DNA-binding heads. The resulting assemblies have appropriate lengths and conformational properties to link sister chromatids in homologous recombination and DNA ends in nonhomologous end-joining.

### CDK4

Human cell division is regulated primarily at the G1-to-S or the G2-to-M boundaries. The sequential activation of cyclin-dependent kinases (CDKs) and their subsequent phosphorylation of critical substrates promote orderly progression through the cell cycle. The complexes formed by CDK4 and the D-type cyclins (e.g., D1; D2; D3) are involved in the control of cell proliferation during the G1 phase. CDK4 is inhibited by p16, also known as cyclin-dependent kinase inhibitor-2 (CDKN2A).

Harbour et al., Cell 98:859-869 (1999), which is hereby incorporated by reference in its entirety, presented evidence that phosphorylation of the C-terminal region of RB by CDK4/CDK6 initiates successive intramolecular interactions between the C-terminal region and the central pocket. The initial interaction displaces histone deacetylase from the pocket, blocking active transcriptional repression by RB. This facilitates a second interaction that leads to phosphorylation of the pocket by CDK2 and disruption of pocket structure. These intramolecular interactions provide a molecular basis for sequential phosphorylation of RB by CDK4/CDK6 and CD 2. CDK4/CDK6 is activated early in G1, blocking active repression by RB. However, it is not until near the end of G1, when cyclin E is expressed and CDK2 is activated, that RB is prevented from binding and inactivating E2F.

Modiano et al., J. Immun. 165:6693-6702 (2000), which is hereby incorporated by reference in its entirety, found that 5 of 16 healthy individuals expressed CDK4 mRNA, protein, and activity in unstimulated peripheral blood T cells and that these T cells proliferated directly in response to interleukin-2 (IL2) in the absence of mitogens. In cells from these individuals, CDK4 expression and activity were resistant to protein kinase inhibitors, unlike stimulated cells from individuals lacking basal CDK4 expression. The phenotype of the T cells of these individuals was comparable to that observed in a human IL2-dependent T-cell line. Modiano et al. (2000) proposed that CDK4 activity may be a useful marker for cytokine responsiveness in T cells.

In primary epidermal cells, Lazarov et al., Nature Med. 8:1105-1114 (2002), which is hereby incorporated by reference in its entirety, found that oncogenic RAS transiently decreases CDK4 expression in association with cell cycle arrest in the G1 phase. CDK4 coexpression circumvents RAS growth suppression and induces invasive human neoplasia resembling squamous cell carcinoma. Tumorigenesis is dependent on CDK4 kinase function, with cyclin D1 required but not sufficient for this process. In facilitating escape from G1 growth restraints, RAS and CDK4 alter the composition of cyclin D and cyclin E complexes and promote resistance to growth inhibition by INK4 cyclin-dependent kinase inhibitors. These data identified a new role for oncogenic RAS in CDK4 regulation and highlighted the functional importance of CDK4 suppression in preventing uncontrolled growth.

Wolfel et al., Science 269:1281-1284 (1995), which is hereby incorporated by reference in its entirety, identified a mutated CDK4 as a tumor-specific antigen recognized by autologous cytolytic T lymphocytes in a human melanoma. The mutated CDK4 allele was present in autologous cultured melanoma cells and metastasis tissue, but not in the patient's lymphocytes. The mutation, an arg24-to-cys (R24C) exchange, was part of the CDK4 peptide recognized by cytolytic T lymphocytes and prevented binding of the CDK4 inhibitor p16(INK4A), but not of p21 or of p27. The same mutation was found in 1 additional melanoma among 28 melanomas analyzed. These results suggested to the authors that mutation of CDK4 can create a tumor-specific antigen and can disrupt the cell cycle regulation exerted by the tumor suppressor p16. Inactivating mutations of the p16 gene are responsible for genetic predisposition to melanoma. The R24C mutation of CDK4 presumably contributes to malignant transformation in melanoma in addition to creating a tumor-specific antigen. Such antigens are ideally suited as targets of tumor rejection responses. The authors speculated that this may have happened in the first patient in whom it was identified, because the patient had remained free of detectable disease for 7 years.

Zou et al., Genes Dev. 16: 2923-2934 (2002), which is hereby incorporated by reference in its entirety, noted that Cdk4 null mice are viable, but they exhibit diabetes mellitus due to degeneration of pancreatic beta cells, as well as growth retardation and infertility due to severe hypoplasia and dysfunction of the pituitary. Embryonic fibroblasts from Cdk4 null mice initially proliferate at normal rates, but they display a 4- to 5-hour delay in reentry into the cell cycle following quiescence. Zou et al. (2002) found that Cdk4 was required for Ras-mediated transformation, and Cdk4 disruption led to senescence that was independent of Arf or p53. Senescence was associated with increased Cdkn1a stability.

### CDC20

Weinstein et al., Molec. Cell Biol. 14: 3350-3363 (1994), which is hereby incorporated by reference in its entirety, identified a protein, designated p55CDC or CDC20, that is homologous to the S. cerevisiae cell division cycle 20 protein, in cycling mammalian cells. This transcript is detectable in all exponentially growing cell lines but disappears when cells are chemically induced to differentiate. The p55CDC protein is essential for cell division. Immunoprecipitation of p55CDC yielded protein complexes with kinase activity that fluctuated during the cell cycle. Since p55CDC did not have the conserved protein kinase domains, this activity must be due to one or more of the associated proteins in the immune complex. The highest levels of protein kinase activity were seen with alpha-casein and myelin basic protein as substrates and demonstrated a pattern of activity distinct from that described for the known cyclin-dependent cell division kinases. The p55CDC protein was also phosphorylated in dividing cells. The 499-amino acid sequence of p55CDC contains 7 repeats homologous to the beta subunit of G proteins. The highest degree of homology in these repeats was found with the S. cerevisiae Cdc20 and Cdc4 proteins, which have been proposed to be involved in the formation of a functional bipolar mitotic spindle in yeast cells. The G beta repeat has been postulated to mediate protein-protein interactions and, in p55CDC, may modulate its association with a unique cell cycle protein kinase.

CDC20 is a component of the mammalian cell cycle mechanism. Activation of the anaphase-promoting complex (APC) is required for anaphase initiation and for exit from mitosis. Fang et al., Molec. Cell 2:163-171 (1998), which is hereby incorporated by reference in its entirety, showed that APC was activated during mitosis and G1 by 2 regulatory factors, CDC20 and HCDH1. These proteins directly bind to APC and activate its cyclin ubiquitination activity. CDC20 confers a strict destruction-box (D-box) dependence on APC, while HCDH1 shows a much more relaxed specificity for the D-box. In HeLa cells, the protein levels of CDC20 as well as its binding to APC peak in mitosis and decrease drastically at early G1. Thus, CDC20 is the mitotic activator of APC and directs the degradation of substrates containing the D-box.

By investigating the essential role of CKS1 in S. cerevisiae, Morris et al., Nature 423:1009-1013 (2003), which is hereby incorporated by reference in its entirety, demonstrated that the protein is primarily involved in promoting mitosis by modulating the transcriptional activation of CDC20. CKS1 is required for both the periodic dissociation of CDC28 kinase from the CDC20 promoter and the periodic association of the proteasome with the promoter. Morris et al. (2003) proposed that the essential role of CKS1 is to recruit the proteasome to, and/or dissociate the CDC28 kinase from, the CDC20 promoter, thus facilitating transcription by remodeling transcriptional complexes or chromatin associated with the CDC20 gene.

Luo et al., Molec. Cell 9:59-71 (2002), which is hereby incorporated by reference in its entirety, showed that RNA interference-mediated suppression of MAD1 function in mammalian cells caused loss of MAD2 kinetochore localization and impairment of the spindle checkpoint. MAD1 and CDC20 contain MAD2-binding motifs that share a common consensus, and the authors identified a class of MAD2-binding peptides (MBPs) with a similar consensus. Binding of one of these ligands, MBP1, triggered an extensive rearrangement of the tertiary structure of MAD2. MAD2 also underwent a similar striking structural change upon binding to a MAD1 or CDC20 binding motif peptide. These data suggested that, upon checkpoint activation, MAD1 recruits MAD2 to unattached kinetochores and may promote binding of MAD2 to CDC20.

### RPL13A

Adams et al., Hum. Molec. Genet. 1:91-96 (1992), which is hereby incorporated by reference in its entirety, identified a novel cDNA representing an mRNA showing significantly higher levels of expression in benign breast lesions than in carcinomas. In both tissues, the expression was highest in epithelial cells as determined by in situ hybridization to tissue sections. The protein deduced from the nucleotide sequence was highly basic with no signal or transmembrane sequence, but 2 potential nuclear localization signals. No significant homology was found with known DNA or protein sequences. The cDNA hybridized to multiple sequences within both human and other mammalian genomes and to single genomic sequences in Drosophila, Physarum, and Schizosaccharomyces pombe. Thus the cDNA represents a highly conserved gene sequence. Only one major transcript was identified in human cells, but the existence of several pseudogenes was suspected.

Thus, one aspect of the present invention relates to methods of modifying cell cycle or cell signaling pathways. These are achieved by modifying the nuclear or cellular concentration of BVR (or fragments or variants thereof) in a cell, whereby an increase in the nuclear concentration of biliverdin reductase, or fragments or variants thereof, increases the expression levels of certain cell cycle or cell signaling proteins and decreases the expression levels of other cell cycle or cell signaling proteins; whereas a decrease in the nuclear or cellular concentration of biliverdin reductase, or fragments or variants thereof, decreases the expression levels of certain cell cycle or cell signaling proteins and increases the expression levels of other cell cycle or cell signaling proteins. These are described in Examples 4 and 5 infra.

Altering the expression level of cell cycle or cell signaling proteins by modifying the nuclear or cellular concentration of BVR (or fragments or variants thereof) in a cell may implicate various diseases. For example, dysregulation of apoptosis can lead to various diseases and disorders. It is now well-known that reduced apoptosis may contribute to tumorigenesis and formation of cancer. Thus, induction of tumor cell apoptosis can be an effective approach in treating cancer. In addition, stimulation of endothelial cell apoptosis may prevent tumor blood supply and cause tumor regression. See Dimmeler and Zeiher, Cir. Res., 87:434-439 (2000), which is hereby incorporated by reference in its entirety. Dysregulation of apoptosis is also an integral part of a wide range of autoimmune diseases and disorders. See Ravirajan et al., Int. Rev. Immunol., 18:563-589 (1999), which is hereby incorporated by reference in its entirety. In addition, many neurological disorders involve apoptosis. During adulthood, there is little normal neuronal cell death. However, neurological diseases, particularly neurodegenerative diseases are often associated with excessive neural cell death. See Honig and Rosenberg, Am. J. Med., 108:317-330 (2000), which is hereby incorporated by reference in its entirety. For example, Parkinson's disease is associated with the loss of substantia nigra pars compacta and sympathetic ganglia, while Alzheimer's disease is characterized with selective cell loss of entorhinal neurons, and hippocampal neurons, cortical neurons. See Honig and Rosenberg, Am. J. Med., 108:317-330 (2000), which is hereby incorporated by reference in its entirety.

Apoptosis also plays an important role in osteoporotic disorders including, but not limited to, postmenopausal osteoporosis, involutional osteoporosis, and glucocorticoid-induced osteoporosis. See Weinstein, et al., Am. J. Med., 108:153-164 (2000), which is hereby incorporated by reference in its entirety. Generally, under normal conditions, the balance between bone formation, bone resorption, bone cell proliferation and apoptosis maintains nearly constant bone mass. The imbalance of such processes leads to abnormal bone remodeling, and thus osteoporosis and other bone-related diseases. It has been suggested that treatment or prevention of osteoporosis may be achieved by promotion of osteoclast apoptosis and prevention of osteoblast apoptosis. See Weinstein, et al., Am. J. Med., 108:153-164 (2000), which is hereby incorporated by reference in its entirety.

Apoptosis also has physiological significance in animal virus infection. See Kyama et al., Microbes and Infection, 2:1111-1117 (2000), which is hereby incorporated by reference in its entirety. Apoptosis of cells infected with viruses may slow the viral multiplication process, although animal viruses typically are able to escape apoptosis of the infected cells. However, it has been suggested that apoptosis of the infected cells triggers the phagocytosis of the dying cells by macrophages. This phagocytosis prevents the leakage of toxic substances that are mediators of dysregulated inflammatory reactions. As a result, dysregulated inflammatory reactions are prevented while specific immune response against the viruses are initiated at the viral infection site. See Kyama et al., Microbes and Infection, 2:1111-1117 (2000), which is hereby incorporated by reference in its entirety. On the other hand, in the case of HIV infection, viral infection-induced apoptosis of CD4⁺ T cells contributes to the depletion of CD4⁺ T cells and progression of HIV infection and AIDS, which is associated with immunodeficiency. Thus, inhibition of apoptosis of CD4⁺ T cells may be a strategy in preventing or treating HIV infection and AIDS. See Kirschner et al., JAIDS J. Acq. Imm. Def Syn., 24:352-362 (2000), which is hereby incorporated by reference in its entirety.

Additionally, apoptosis also plays a role in diseases such as ischemic heart disease, stroke, and sepsis. For example, apoptosis-related neuronal cell death after cerebral ischemia may contribute to stroke. See Johnson et al., J. Neurotrauna., 12:843-52 (1995), which is hereby incorporated by reference in its entirety. Thus, inhibition of apoptosis may be an approach in the development of therapeutic interventions of ischemic stroke. In addition, the inhibition of endothelial cell apoptosis may improve angiogenesis and vasculogenesis in patients with ischemia, and thus may be an effective method for treating ischemia injuries. See Dimmeler and Zeiher, Cir. Res., 87:434-439 (2000), which is hereby incorporated by reference in its entirety.

Thus, the methods can be applicable to a variety of tumors, i.e., abnormal growth, whether cancerous (malignant) or noncancerous (benign), and whether primary tumors or secondary tumors. Such disorders include but are not limited to lung cancers such as bronchogenic carcinoma (e.g., squamous cell carcinoma, small cell carcinoma, large cell carcinoma, and adenocarcinoma), alveolar cell carcinoma, bronchial adenoma, chondromatous hamartoma (noncancerous), and sarcoma (cancerous); heart tumors such as myxoma, fibromas and rhabdomyomas; bone tumors such as osteochondromas, condromas, chondroblastomas, chondromyxoid fibromas, osteoid osteomas, giant cell tumors, chondrosarcoma, multiple myeloma, osteosarcoma, fibrosarcomas, malignant fibrous histiocytomas, Ewing's tumor (Ewing's sarcoma), and reticulum cell sarcoma; brain tumors such as gliomas (e.g., glioblastoma multiforme), anaplastic astrocytomas, astrocytomas, and oligodendrogliomas, medulloblastomas, chordoma, Schwannomas, ependymomas, meningiomas, pituitary adenoma, pinealoma, osteomas, and hemangioblastomas, craniopharyngiomas, chordomas, germinomas, teratomas, dennoid cysts, and angiomas; various oral cancers; tumors in digestive system such as leiomyoma, epidermoid carcinoma, adenocarcinoma, leiomyosarcoma, stomach adenocarcinomas, intestinal lipomas, intestinal neurofibromas, intestinal fibromas, polyps in large intestine, familial polyposis such as Gardner's syndrome and Peutz-Jeghers syndrome, colorectal cancers (including colon cancer and rectal cancer); liver cancers such as hepatocellular adenomas, hemangioma, hepatocellular carcinoma, fibrolamellar carcinoma, cholangiocarcinoma, hepatoblastoma, and angiosarcoma; kidney tumors such as kidney adenocarcinoma, renal cell carcinoma, hypemephroma, and transitional cell carcinoma of the renal pelvis; bladder cancers; tumors in blood system including acute lymphocytic (lymphoblastic) leukemia, acute myeloid (myelocytic, myelogenous, myeloblastic, myelomonocytic) leukemia, chronic lymphocytic leukemia (e.g., Sezary syndrome and hairy cell leukemia), chronic myelocytic (myeloid, myelogenous, granulocytic) leukemia, Hodgkin's lymphoma, non-Hodgkin's lymphoma, mycosis fungoides, and myeloproliferative disorders (including myeloproliferative disorders are polycythemia vera, myelofibrosis, thrombocythemia, and chronic myelocytic leukemia); skin cancers such as basal cell carcinoma, squamous cell carcinoma, melanoma, Kaposi's sarcoma, and Paget's disease; head and neck cancers; eye-related cancers such as retinoblastoma and intraocular melanocarcinoma; male reproductive system cancers such as benign prostatic hyperplasia, prostate cancer, and testicular cancers (e.g., seminoma, teratoma, embryonal carcinoma, and choriocarcinoma); breast cancer; female reproductive system cancers such as uterus cancer (endometrial carcinoma), cervical cancer (cervical carcinoma), cancer of the ovaries (ovarian carcinoma), vulvar carcinoma, vaginal carcinoma, fallopian tube cancer, and hydatidiform mole; thyroid cancer (including papillary, follicular, anaplastic, or medullary cancer); pheochromocytomas (adrenal gland); noncancerous growths of the parathyroid glands; cancerous or noncancerous growths of the pancreas; etc.

Specifically, breast cancers, colon cancers, prostate cancers, lung cancers and skin cancers may be amenable to the treatment by the methods of the present invention. In addition, premalignant conditions may also be treated by the methods of the present invention to prevent or stop the progression of such conditions towards malignancy, or cause regression of the premalignant conditions. Examples of premalignant conditions include hyperplasia, dysplasia, and metaplasia.

Thus, the term “treating cancer” as used herein, specifically refers to administering therapeutic agents to a patient diagnosed of cancer, i.e., having established cancer in the patient, to inhibit the finther growth or spread of the malignant cells in the cancerous tissue, and/or to cause the death of the malignant cells. The term “treating cancer” also encompasses treating a patient having premalignant conditions to stop the progression of, or cause regression of, the premalignant conditions.

The methods of the present invention may also be useful in treating or preventing other diseases and disorders caused by abnormal cell proliferation (hyperproliferation or dysproliferation), e.g., keloid, liver cirrhosis, psoriasis, etc. In addition, the methods may also find applications in promoting wound healing, and other cell and tissue growth-related conditions.

The methods for modulating the expression levels of cell cycling and cell signaling proteins may be employed to modulate apoptosis and lipid metabolism. In addition, the methods may also be used in the treatment or prevention of diseases and disorders such as cancer, viral infection, AIDS, asthma, ischemia, stroke, autoimmune diseases, neurodegenerative diseases, inflammatory disorders, sepsis, and osteoporosis.

In yet another embodiment, the methods for modulating the expression levels of cell cycling and/or cell signaling proteins may be used in treating or preventing autoimmune diseases and disorders including, but not limited to, rheumatoid arthritis, systemic lupus erythematosus (SLE), Sjogren's syndrome, Canale-Smith syndrome, psoriasis, scleroderma, dennatomyositis, polymyositis, Behcet's syndrome, skin-related autoimmue diseases such as bullus pemphigoid, IgA dermatosis, pemphigus vulgaris, pemphigus foliaceus, dermatitis herpetifonnis, contact dermatitis, autoimmune allopecia, erythema nodosa, and epidermolysis bullous aquisita, drug-induced hemotologic autoimmune disorders, autoimmue thrombocytopenic purpura, autoimmune neutropenia, systemic sclerosis, multiple sclerosis, imflammatory demyelinating, diabetes mellitus, autoimmune polyglandular syndromes, vasculitides, Wegener's granulomatosis, Hashimoto's disease, multinodular goitre, Grave's disease, autoimmune encephalomyelitis (EAE), demyelinating diseases, etc.

The methods of the present invention can also be useful in treating neurodegenerative disorders including, but not limited to, Alzheimer's disease, frontotemporal dementia, Parkinson's disease, Huntington's disease, brain trauma, infarction, hemorrhage, amytrophic lateral sclerosis/Lou Gehrig's disease (ALS), inherited ataxias such as olivopontocerebellar atrophy (spinocerebellar ataxia type 1), and Machado-Joseph disease (spinocerebellar ataxia type 3).

BVR can be used for therapeutic interventions in neurodegenerative disorders as a method to promote neuronal cell growth or differentiation of uncommited cells to neurons. BVR also can be used to control viral replication and oncogensis.

The cell in which the nuclear or cellular concentration of BVR, or fragments or variants thereof, is to be modified can be located in vivo or ex vivo. The modification of BVR nuclear or cellular concentrations can be also be used as one part of a multi-component approach for treated diseases or disorders that implicate the cell cycle or cell signaling proteins whose expression levels can be modified by BVR. Such complimentary treatments can be any suitable therapy, whether now known or hereafter developed.

The nuclear or cellular concentration of BVR (or fragments or variants thereof) can be modified according to a number of approaches, either by delivering the BVR (or fragments or variants thereof) or antisense BVR RNA molecule or siRNA into the cell in a manner which affords the protein or polypeptide or RNA molecule to be active within the cell, or by delivering DNA encoding BVR (or fragments or variants thereof) or antisense BVR RNA molecule or siRNA into the cell in a manner effective to induce the expression thereof in the cell. When BVR (or fragments or variants thereof) is delivered into target cells, it may be desirable that such delivery be effective to cause nuclear uptake of the BVR (or fragments or variants thereof). As noted above, BVR or fragments or variants contain the native BVR nuclear localization signal or a chimeric nuclear localization signal. When antisense BVR RNA or siRNA is delivered into target cells, the antisense RNA or siRNA is effective in the cytoplasm and need not be targeted to any particular location within the cytoplasm, although higher efficacy can be obtained when targeting the antisense BVR RNA to ribosomal sites.

One approach for delivering protein or polypeptides or RNA molecules into cells involves the use of liposomes. Basically, this involves providing a liposome which includes that protein or polypeptide or RNA to be delivered, and then contacting the target cell with the liposome under conditions effective for delivery of the protein or polypeptide or RNA into the cell.

Liposomes are vesicles comprised of one or more concentrically ordered lipid bilayers which encapsulate an aqueous phase. They are normally not leaky, but can become leaky if a hole or pore occurs in the membrane, if the membrane is dissolved or degrades, or if the membrane temperature is increased to the phase transition temperature. Current methods of drug delivery via liposomes require that the liposome carrier ultimately become permeable and release the encapsulated drug at the target site. This can be accomplished, for example, in a passive manner wherein the liposome bilayer degrades over time through the action of various agents in the body. Every liposome composition will have a characteristic half-life in the circulation or at other sites in the body and, thus, by controlling the half-life of the liposome composition, the rate at which the bilayer degrades can be somewhat regulated.

In contrast to passive drug release, active drug release involves using an agent to induce a permeability change in the liposome vesicle. Liposome membranes can be constructed so that they become destabilized when the environment becomes acidic near the liposome membrane (see, e.g., Proc. Natl. Acad. Sci. USA 84:7851 (1987); Biochemistry 28:908 (1989), each of which is hereby incorporated by reference in its entirety). When liposomes are endocytosed by a target cell, for example, they can be routed to acidic endosomes which will destabilize the liposome and result in drug release.

Alternatively, the liposome membrane can be chemically modified such that an enzyme is placed as a coating on the membrane, which enzyme slowly destabilizes the liposome. Since control of drug release depends on the concentration of enzyme initially placed in the membrane, there is no real effective way to modulate or alter drug release to achieve “on demand” drug delivery. The same problem exists for pH-sensitive liposomes in that as soon as the liposome vesicle comes into contact with a target cell, it will be engulfed and a drop in pH will lead to drug release.

This liposome delivery system can also be made to accumulate at a target organ, tissue, or cell via active targeting (e.g., by incorporating an antibody or hormone on the surface of the liposomal vehicle). This can be achieved according to known methods.

Different types of liposomes can be prepared according to Bangham et al., J. Mol. Biol. 13:238-252 (1965); U.S. Pat. No. 5,653,996 to Hsu et al.; U.S. Pat. No. 5,643,599 to Lee et al.; U.S. Pat. No. 5,885,613 to Holland et al.; U.S. Pat. No. 5,631,237 to Dzau et al.; and U.S. Pat. No. 5,059,421 to Lougbrey et al., each of which is hereby incorporated by reference in its entirety.

An alternative approach for delivery of proteins or polypeptides involves the conjugation of the desired protein or polypeptide to a polymer that is stabilized to avoid enzymatic degradation of the conjugated protein or polypeptide. Conjugated proteins or polypeptides of this type are described in U.S. Pat. No. 5,681,811 to Ekwuribe, which is hereby incorporated by reference in its entirety.

Yet another approach for delivery of proteins or polypeptides involves preparation of chimeric proteins according to U.S. Pat. No. 5,817,789 to Heartlein et al., which is hereby incorporated by reference in its entirety. The chimeric protein can include a ligand domain and, e.g., BVR or a fragment or variant thereof as described above. The ligand domain is specific for receptors located on a target cell. Thus, when the chimeric protein is delivered intravenously or otherwise introduced into blood or lymph, the chimeric protein will adsorb to the targeted cell, and the targeted cell will internalize the chimeric protein.

When it is desirable to achieve heterologous expression of a desirable protein or polypeptide or RNA molecule in a target cell, DNA molecules encoding the desired protein or polypeptide or RNA can be delivered into the cell. Basically, this includes providing a nucleic acid molecule encoding the protein or polypeptide and then introducing the nucleic acid molecule into the cell under conditions effective to express the protein or polypeptide or RNA in the cell. Preferably, this is achieved by inserting the nucleic acid molecule into an expression vector before it is introduced into the cell.

When transforming mammalian cells for heterologous expression of a protein or polypeptide, an adenovirus vector can be employed. Adenovirus gene delivery vehicles can be readily prepared and utilized given the disclosure provided in Berkner, Bioteclhniques 6:616-627 (1988) and Rosenfeld et al., Science 252:431-434 (1991), WO 93/07283, WO 93/06223, and WO 93/07282, each of which is hereby incorporated by reference in its entirety. Adeno-associated viral gene delivery vehicles can be constructed and used to deliver a gene to cells. The use of adeno-associated viral gene delivery vehicles in vitro is described in Chatteijee et al., Science 258:1485-1488 (1992); Walsh et al., Proc. Nat'l. Acad. Sci. USA 89:7257-7261 (1992); Walsh et al., J. Clin Invest. 94:1440-1448 (1994); Flotte et al., J. Biol. Chem. 268:3781-3790 (1993); Ponnazhagan et al., J. Exp. Med. 179:733-738 (1994); Miller et al., Proc. Nat'l Acad. Sci. USA 91:10183-10187 (1994); Einerhand et al., Gene Ther. 2:336-343 (1995); Luo et al., Exp. Hematol. 23:1261-1267 (1995); and Zhou et al., Gene Ther. 3:223-229 (1996), each of which is hereby incorporated by reference in its entirety. In vivo use of these vehicles is described in Flotte et al., Proc. Nat'l Acad. Sci. USA 90:10613-10617 (1993); and Kaplitt et al., Nature Genet. 8:148-153 (1994), each of which is hereby incorporated by reference in its entirety. Additional types of adenovirus vectors are described in U.S. Pat. No. 6,057,155 to Wickham et al.; U.S. Pat. No. 6,033,908 to Bout et al.; U.S. Pat. No. 6,001,557 to Wilson et al.; U.S. Pat. No. 5,994,132 to Chamberlain et al.; U.S. Pat. No. 5,981,225 to Kochanek et al.; and U.S. Pat. No. 5,885,808 to Spooner et al.; and U.S. Pat. No. 5,871,727 to Curiel, each of which is hereby incorporated by reference in its entirety).

Retroviral vectors which have been modified to form infective transformation systems can also be used to deliver nucleic acid encoding a desired protein or polypeptide or RNA product into a target cell. One such type of retroviral vector is disclosed in U.S. Pat. No. 5,849,586 to Kriegler et al., which is hereby incorporated by reference in its entirety.

Regardless of the type of infective transformation system employed, it should be targeted for delivery of the nucleic acid to a specific cell type. For example, for delivery of the nucleic acid into a cluster of cells, a high titer of the infective transformation system can be injected directly within the site of those cells so as to enhance the likelihood of cell infection. The infected cells will then express the desired product, in this case BVR (or fragments or variants thereof) or antisense BVR RNA, to modify the expression of cell cycle or cell signaling proteins.

Whether the proteins or polypeptides or nucleic acids are administered alone or in combination with pharmaceutically or physiologically acceptable carriers, excipients, or stabilizers, or in solid or liquid form such as, tablets, capsules, powders, solutions, suspensions, or emulsions, they can be administered orally, parenterally, subcutaneously, intravenously, intramuscularly, intraperitoneally, by intranasal instillation, by intracavitary or intravesical instillation, intraocularly, intraarterially, intralesionally, by application to mucous membranes, such as, that of the nose, throat, and bronchial tubes, or by transdermal delivery. For most therapeutic purposes, the proteins or polypeptides or nucleic acids can be administered intravenously.

For injectable dosages, solutions or suspensions of these materials can be prepared in a physiologically acceptable diluent with a pharmaceutical carrier. Such carriers include sterile liquids, such as water and oils, with or without the addition of a surfactant and other pharmaceutically and physiologically acceptable carrier, including adjuvants, excipients or stabilizers. Illustrative oils are those of petroleum, animal, vegetable, or synthetic origin, for example, peanut oil, soybean oil, or mineral oil. In general, water, saline, aqueous dextrose and related sugar solution, and glycols, such as propylene glycol or polyethylene glycol, are preferred liquid carriers, particularly for injectable solutions.

For use as aerosols, the proteins or polypeptides or nucleic acids in solution or suspension may be packaged in a pressurized aerosol container together with suitable propellants, for example, hydrocarbon propellants like propane, butane, or isobutane with conventional adjuvants. The materials of the present invention also may be administered in a non-pressurized form such as in a nebulizer or atomizer.

Both the biliverdin reductase, or fragment or variant thereof, and the antisense RNA can be delivered to the target cells (i.e., at or around the site of the stroke/ischemic event) using the above-described methods for delivering such therapeutic products. In delivering the therapeutic products to nerve cells in the brain, consideration should be provided to negotiation of the blood-brain barrier. The blood-brain barrier typically prevents many compounds in the blood stream from entering the tissues and fluids of the brain. Nature provides this mechanism to insure a toxin-free environment for neurologic function. However, it also prevents delivery to the brain of therapeutic compounds.

One approach for negotiating the blood-brain barrier is described in U.S. Pat. No. 5,752,515 to Jolesz et al., which is hereby incorporated by reference in its entirety. Basically, the blood-brain barrier is temporarily “opened” by targeting a selected location in the brain and applying ultrasound to induce, in the central nervous system (CNS) tissues and/or fluids at that location, a change detectable by imaging. A protein or polypeptide or RNA molecule of the present invention can delivered to the targeted region of the brain while the blood-brain barrier remains “lopen,” allowing targeted neuronal cells to uptake the delivered protein or polypeptide or RNA. At least a portion of the brain in the vicinity of the selected location can be imaged, e.g., via magnetic resonance imaging, to confirm the location of the change. Alternative approaches for negotiating the blood-brain barrier include chimeric peptides and modified liposome structures which contain a PEG moiety (reviewed in Pardridge, J. Neurochem. 70:1781-1792 (1998), which is hereby incorporated by reference in its entirety), as well as osmotic opening (i.e., with bradykinin, mannitol, RPM7, etc.) and direct intracerebral infusion (Kroll et al., Neurosurgeiy 42(5):1083-1100 (1998), which is hereby incorporated by reference in its entirety.

### EXAMPLES

The following examples are provided to illustrate embodiments of the present invention, but they are by no means intended to limit its scope.

### Example 1

### Construction of Adenoviral Vector Expressing hBVR

Adenovirus recombinant DNAs with hBVR were constructed as follows. Firstly, full-length biliverdin reductase cDNA was amplified from the clone obtained earlier in the laboratory (Maines et al., Eur. J. Biochem. 235:372-381 (1996), which is hereby incorporated by reference in its entirety) using primers 724BVR (5′-GTC ACG AGA TCT CGA TTA TTA GGA CGA TGA CGA TAA GAT GAA TGC AGA GCC CGA GAG GAA GTT TGG CG) (SEQ ID NO: 10) and 725BVR (5′-GTC ACG TCT AGA TTA CTT CCT TGA ACA GCA ATA TTT CTG GAT TTC TGC)* (SEQ ID NO: 11). Primer 724BVR allows to introduce FLAG (DYKDDDDK) (SEQ ID NO: 12) coding sequence just upstream from ATG codon of hBVR cDNA. The resulting fragment was digested with BglII and XbaI restriction endonucleases and cloned between the appropriate sites of vector plasmid pEGFP-3C. The cDNA sequence of hBVR was verified by sequencing.

For transformation, dam⁻ strain GM119 was used (kindly provided by S. Hattman, Department of Biology, University of Rochester), since the XbaI site in resulting construct named pGFP-hBVR was protected by Dam methylation when maintained in non-modified bacterial recipient. The NheI-XbaI fragment of pGFP-hBVR contained fusion EGFP-FLAG-hBVR was subcloned between the sites NheI-XbaI of intermediate vector pTRE-Shuttle2. The fact that NheI and XbaI restriction endonucleases produce identical cohesive ends allowed us to obtained constructs with two alternative orientations of the fragment: one under control of the tetracycline regulated plasmid promoter P_{minCMV} (pTRE-hBVR), the other in the opposite direction (pTRE-INV-hBVR). After thorough sequence analysis, both constructs were used for subdloning into tetracycline-responsive pAdeno-X vector according to the company's instructions. Recombinant DNA's named correspondently Ad-hBVR and AD-INV-hBVR were purified from XL1-Blue Gold recipient bacterial strain and analyzed with restriction endonucleases and PCR using gene specific primers and primers provided by Clontech to check orientation of the insert. Finally, Ad-hBVR and AD-INV-hBVR were introduced into HEK 293A cells using Lipofectamin 2000 protocol (Invitrogen, Carlsbad, Calif.). The viruses were isolated from cell culture by AdenoPure (Puresyn, Malvern, Pa.) purification kit according to the supplier's recommendations. The viral titer was determined by OD₂₆₀ assay in accordance with BD Biosciences Clontech's protocol.

### Example 2

### Cell Culture and Transfection of hBVR Into 293A Cells

293A cell line (a human embryonal kidney cell line) was obtained from ATCC (Rockville, Md.). Cells (3×10⁶ for RNA analysis and 1×10⁶ for protein analysis) were grown in Dulbecco's Modified Eagle Medium (D-MEM) containing 10% tetracycline-free fetal bovine serum and 1% penicillin-G/streptomycin for 18 hours. Then, virus was added at a multiplicity of infection 5 pfa/cell for Adeno-X Tet-On and 10 pfu/cell for two recombinant constructs. This ratio was found to be optimal for overexpression of hBVR. For some analyses, upon the addition of the virus, cells were collected and used as controls. For most experiments, 2 h after the addition of the virus, Dox was added at a concentration of 5 μg/ml. This time point was designated in figures as the 0 point. Samples were collected at time points indicated in figures.

### Example 3

### Northern Blot Analysis

RNA was extracted with an RNeasy kit (Qiagen, Valencia, Calif.) from 293A line cells infected or non-infected with Ad-hBVR or Ad-INV-hBVR. RNA was separated by electrophoresis on denaturating formaldehyde gel and transferred onto Hybond membrane. The membrane were probed for hBVR, (fall length hBVR cDNA (Hoeffler et al., Science 242:1430-1433 (1988), which is hereby incorporated by reference in its entirety), hHO1-1 (0.8 kb fragment of hHO-1 cDNA (Shibahara et al., Proc. Natl. Sci. USA 82:7865-7869 (1985), which is hereby incorporated by reference in its entirety), ATF-2, XbaI/HincII fragment of pMT2-HA-ATF-2 plasmid (generous gift from Dr. van Dam, Netherlands) containing human ATF-2 cDNA, GAPDH (1.3 kb fragment of GAPDH cDNA), and human beta-actin (1.1 kb fragment of human-actin cDNA). Probes were labeled using [α-³²P]dCTP with the random primers labeling system (Gibco, Carlsbad, Calif.). Pre-hybridization, hybridization and autoradiography were performed as described previously (Miralem et al., Mol. Cell. Biol. 23:579-593 (2003); which is hereby incorporated by reference in its entirety).

### Example 4

### cDNA Microarray in 293 Cells

Total RNA (20 μg) was extracted from 5×10⁶ 293A line cells infected with Ad-hBVR (10 pfu/cell) and Adeno-X Tet-On (5 pfu/cell) or Ad-INV-hBVR (10 pfu/cell) and Adeno-X Tet-On (5 pfu/cell) 24 h after induction with 5 μg/μl Doxycycline was used for cDNA microarray analysis by SuperArray (Frederick, Md.). After cDNA synthesis and Biotin d-UTP labeling, the probes were hybridized with GEArray Q Series Human Signal Transduction PathwayFinder Gene Array HS-008 membrane containing cDNA fragments from 96 marker genes associated with 18 signal transduction pathways. Analysis of the arrays was performed with “AlphaEasy” software together with “AlphaImager™ 3400” system (Alpha Innotech Corp., San Leandro, Calif.) to convert signal intensity to a nunmeric number. All signal intensities were nonnalized to housekeeping genes β-actin, GAPDH, cyclophilin A and ribosomal protein L13α. Changes in genes expression were calculated as the ratio between intensities of signals after overexpressing wild type (Ad-hBVR) and inverted (Ad-INV-hBVR, control) adenoviral constructs during the microarray analysis giving a fold of increase/decrease: Ad-hBVR/Ad-INV-hBVR. Any value over 2-fold change was taken as a significant and is presented in Table 1 (below) using the same criteria, any significantly suppressed gene was indicated as negative (−) fold value.

TABLE 1
Microarray analysis influenced by hBVR overexpression in 293 Cells

| Gene Symbol | foldΔ |
|---|---|
| CYP19A1 | 36.0 |
| CSN2 | 9.2 |
| ATF-2 | 8.4 |
| GADD45 | 7.6 |
| HSF1 | 7.5 |
| HHIP | 4.5 |
| c-JUN | 3.8 |
| PECAM1 | 3.7 |
| HSPB1 | 3.4 |
| BCL2 | 3.4 |
| EGFR | 3.4 |
| BCL2L1 | 3.1 |
| BIRC2 | 2.6 |
| HSPA | 2.5 |
| PRKCA | 2.4 |
| PTGS2 | 2.3 |
| WISP3 | 2.2 |
| CDKN2B | 0.4 |
| CDKN1B | 0.2 |

The values indicated in the table represent the ratio between intensities of the signal for genes in RNA isolated from cells transfected with wild type (Ad-hBVR) or with inverted (Ad-INV-hBVR). Any value over 2-fold change was taken as significant and presented in the table. Applying the same criteria, (—) indicates significantly suppressed genes.

### Example 5

### Microarray Analysis of Cell Cycle and Cell Signaling Proteins Following BVR Expression in HEK Cells

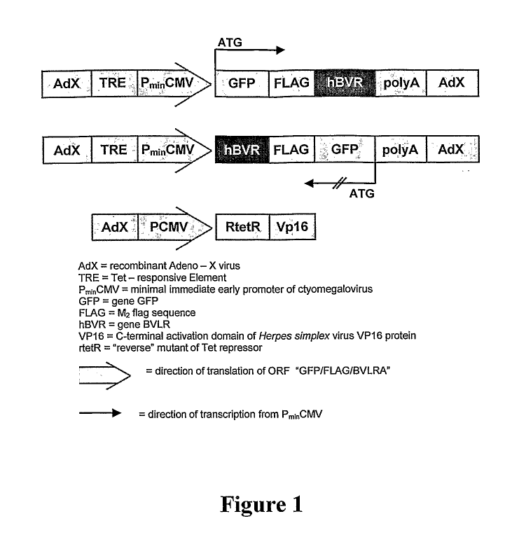

The structure of the coding and the non-coding constructs are illustrated in FIG. 1 and described in Example 1 above. 16 h after transfection there was a significant increase in hBVR expression that reached a prominent peak at 24 h after transfection. Analysis of percent of cells in G1/G0 phase at 18 h and 24 h, respectively, resulted in the following data: 31% and 21% when transfected with the reverse construct, 50% and 48% when transected with the wild-type BVR construct. Because the G1/G0 is the quiescence phase in cell cycle, i.e., when DNA repair takes place, an increase in duration of the phase is considered a protective cell response. The results of the gene array analysis showed increase in mRNA levels for several kinases and transcription factors that control cell cycle transition. The increases measured up to 30-fold were found in: ATF-2/CREB (activating transcription factor-2, cAMP response element binding protein), cyclina, cycline, and E2F-3. Others also demonstrated significant increases in expression levels. Notably, mRNA levels for cytochrome P450 aromatase, an enzyme responsible for estrogen biosynthesis, was increased by over 30-fold. Transcription of this gene is regulated through cAMP regulatory element. Western blot analysis revealed that in fact ATF-2 protein is significantly increased in cells transfected with hBVR at 16 h and 24 h after transfection.

In hBVR transfected cells, gene array analysis show altered expression of several other cell cycle enzymes and regulators including Cdc25a. Cdc25a mRNA is increased by over 20-fold. This cell division enzyme activates Cdc2Cyclin B complex formation that induces meiotic metaphase. These studies show that hBVR interact with chromatin as cell divides.

TABLE 2
Analysis of Cell Cycle and Cell Signal Proteins in HEK Cells

| Gene Name | INV | WT | Fold Change |
|---|---|---|---|
| cyclin A | 8 | 28.4 | 3.55 |
| cyclin B | 18.6 | 12.8 | −0.69 |
| cyclin B2 | 7.8 | 4.8 | −0.62 |
| cyclin E1 | 6.8 | 27 | 3.97 |
| cyclin E2 | 2.6 | 9.2 | 3.54 |
| cyclin G | 32.8 | 17 | −0.52 |
| P55cdc (CDC20) | 1 | 2.6 | 2.60 |
| CDC25a, phosphotase | 2.8 | 44.4 | 15.86 |
| CDC6 | 36.8 | 58 | 1.58 |
| CDC7 | 7.8 | 3.8 | −0.49 |
| cdk2 | 8.2 | 40.4 | 4.93 |
| p16ink4 | 146.4 | 168.6 | 1.15 |
| CKS1p9 | 97.8 | 127.2 | 1.30 |
| CKS2 | 4 | 9 | 2.25 |
| Cullin-Cul2 | 16.4 | 21.6 | 1.32 |
| Cullin-Cul3 | 34.8 | 48.4 | 1.39 |
| E2F-3 | 0.8 | 10.8 | 13.50 |
| MAD2L1 | 12.6 | 23.2 | 1.84 |
| MCM3 | 0.6 | 12.8 | 21.33 |
| MCM6 (Mis5) | 69.8 | 177.6 | 2.54 |
| MCM7 (cdc47) | 3.4 | 16.4 | 4.82 |
| Nedd8 | 42.4 | 26.8 | −0.63 |
| PCNA | 0.2 | 1.4 | 7.00 |
| PRC1 | 22.2 | 39.2 | 1.77 |
| Rbx1 | 5.8 | 50.2 | 8.66 |
| skp1 | 17.8 | 3.8 | −0.21 |
| skp2 | 72.4 | 40.2 | −0.56 |
| SUMO-1 ub (sentrin) | 28.8 | 34.8 | 1.21 |
| b-actin | 36.6 | 26.1 | −0.71 |

The values indicated in the table (under fold change) represent the ratio between intensities of the signal for genes in RNA isolated from cells transfected with wild type (Ad-hBVR) or with inverted (Ad-INV-hBVR).

### Example 6

### Western Blot

Cells infected as above were collected by centrifugation (200 rpm for 5 min at room-temperature), washed with PBS, and resuspended in 100 μl 4× lysis buffer (200 mM Tris-Cl, 57.2 mM beta-mercaptoethanol, 8% SDS, 0.2% bromophenol blue, 40% glycerol).

Cell lysate was subjected to electrophoresis on 14% SDS-polyachrylamide gels and transferred to polyvinylidene fluoride membrane (Pall Corporation, Ann Arbor, Mich.). BVR was detected using rabbit antihuman BVR polyclonal antibodies (3:500, v:v) as the primary antibody (Maines et al., Arch. Biochem. Bioplys. 300:320-326 (1993), which is hereby incorporated by reference in its entirety) and horseradish-peroxidase labeled anti-rabbit IgG (1:5000, v:v Amersham, Piscataway, N.J.) as the secondary antibody. The hBVR protein was visualized by either DAB staining according to manufacturer's instructions (Sigma-Aldrich, St. Louis, Mo.) or by enhanced Chemiluminescence (ECL) detection system (Amersham, Piscataway, N.J.). The FLAG tag was detected by monoclonal mouse anti-FLAG antibodies (Sigina-Aldrich, Milwaukee, Wis,) and visualized by DAB staining. ATF-2 was detected by ATF-2 polyclonal rabbit antibodies (Cell Signaling, Beverly, MA) and anti-phospho-ATF-2 (thr69/71) polyclonal rabbit antibodies (Cell Signaling, Beverly, Mass.). Specific bands were visualized using the ECL system. For BBVR and FLAG tag fusion protein detection, 2 gl of induced cell lysate corresponding to 2.0×10⁴ cells was used. In all other cases, 20 to 40 μl aliquots of cell extract were used.

### Example 7

### ELISA

HO-1 protein was measured using an ELISA kit developed by Stressgen Bioreagents (Victoria, BC, Calif.) according to the manufacturer's instructions.

### Example 8

### Measurement of BVR Activity

293A cells infected with Ad-hBVR or Ad-INV-hBVR were lysed in buffer containing 50 mM Tris-HCl (pH, 7.4), 75 mM NaCl, 20 mM MgCl2, 10 mM MnCl2, 1% Nonidet-P-400, 2 mM EDTA, 2 mM EGTA, 10% glycerol, protease inhibitor cocktail (1 μg/ml approtinin, 1 μg/ml leupeptin, 1 μg/ml pepstatin, and 0.1 mM PMSF) and phosphatase inhibitors (10 mM NaF, 1 mM NaVO4). Non-infected 293A cells were used as a negative control. Cell lysates were briefly sonicated and centrifuged. The hBVR activity was measured in the supernatant fraction at pH 6.7 as described previously (Kutty et al., J. Biol. Chem. 256:3956-3962 (1981); Benbrook et al., Oncogene 5:295-302 (1990); Immenschuh et al., Biochem. J. 334:141-146 (1998), which are hereby incorporated by reference in their entirety). The rate of reduction of biliverdin to bilirubin was determined as the increase in 450 nm absorbance at 25° C. Specific activity is expressed as mnol of bilirubin/min/mg of protein.

### Example 9

### In Vitro BVR Protein Translation, Nuclear Extraction, and Gel Mobility-Shift Assay

Both in vitro translated BVR protein and nuclear extract from 293 cells transfected with Ad hBVR were used in the gel shift assay experiments. In vitro BVR protein translation was performed using a TNT Quick Coupled Translation System from Promega (Madison, Wis.). Briefly, full length hBVR cDNA was cloned into a pcDNA3 expression vector downstream from the T7 RNA polymerase promoter. 2.0 μg of recombinant plasmid DNA obtained was used for protein translation with TNT Quick Master Mix in a 50 μl reaction volume for 90 min at 30° C. Nuclear extract was isolated from 293 cells transfected with Ad-BVR 10⁷ cells were harvested at time points 0, 6 and 24 h after induction of hBVR expression; cells were washed with cold PBS and then lysed with hypotonic buffer. Nuclei were collected by centrifugation at 800×g at 4° C. for 10 min and resuspended in 200 μl of buffer containing 20 mM HEPES pH 7.9, 25% glycerol, 0.4 M NaCl, 1.5 mM MgCl₂, 0.2 mM EDTA, 0.5 M PMSF, 10 mM KCl, 0.5 mM DTT. The nuclei were extracted on ice for 30 min and followed by 30 min centrifugation at 4° C. Supernatant was then collected. The synthesized proteins or nuclear extract were then analyzed in gel shift assay for their DNA binding capability to cAMP regulatory element (CRE) and AP-1 consensus oligos (Angel et al., Biochim. Biophys. Acta. 1072:129-157 (1991), which is hereby incorporated by reference in its entirety). The sequences of oligonucleotides used in the present study are listed in Table 3 (below). The oligos were labeled with [γ-³²P]ATP by T4 kinase (Invitrogen, Carlsbad, Calif.) according to the manufacturer's instructions. For DNA binding assay, 3 ul of in vitro translated protein was preincubated with 2 ul of binding buffer (20% glycerol, 5 mM MgCl₂, 2.5 mM EDTA, 2.5 mM DTT, 250 mM NaCl, and 50 mM Tris-HCl, 0.25 mg/ml poly (dI):poly(dC)) in 8 ul reaction volume for 10 min at room temperature. Then, 2 ul of labeled oligos were added, and sarnples were incubated for an additional 20 min at room temperature. The DNA-protein binding complexes were electrophoresed on a 6% non-denature poly-acrylamide gel and processed for autoradiography. The unlabeled competitor DNA was used to determine the specificity of the binding. In order to identify proteins in the DNA-protein complexes, supershift experiments were performed with rabbit polyclonal anti-hBVR antibodies (Shibahara et al., Proc. Natl. Sci. USA 82:7865-7869 (1985), which is hereby incorporated by reference in its entirety). For positive control, ATF-2 protein was in vitro translated using a pcDNA3 plasmid containing a 762 bp ATF-2 cDNA sequence as template (generous gift from Hicham Drissi, University of Rochester). The translated ATF-2 protein was then used in the CRE binding reaction. Each experiment was repeated at least twice to ascertain the reproducibility of results.

TABLE 3

Oligonucleotide Sequence Used
for Gel Shift Assays

| Name of | |
|---|---|
| Oligos | Sequence of Oligos* |
| 1x CRE | 5′- AGA GAT TGC CTG ACG TCA GAG AGC TAG-3′ |
| | (SEQ ID NO:13) |
| 4x CRE | 5′- (AGC CTG ACG TCA GAG)x4-3′ |
| | (SEQ ID NO:14) |
| 1x mut | 5′- AGA GAT TGC CAT GGC ATC GAG AGC TAG-3′ |
| CRE | (SEQ ID NO:15) |
| | 5′- (nt106) AAT AGT GAC TAG TTT TGG GG [symbol] |
| | AGT (nt 120) ATA A [symbol] ACT |
| | TAT G-3′ |
| | (SEQ ID NO:16) |

*Only the upper strands of double-stranded fragments are given.
**The DNA fragment with three potential AP-1 binding sites as predicated by Malinspector software. The double underlined sequence has a similarity of 94% with AP-1 consensus sequence (TG/TAGTCA); the other two dotted, and underlined sequences have a similarity of 87% and 86% respectively. The initiation ATG codon of ATF-2 original ORF is shown in bold.

### Example 10

### Luciferase Assays

Transfection of 293 cells in 24 well plates was performed by using Lipofectarnine (Invitrogen, Carlsbad, Calif.) with 0.4 μg of the ATF-2 promoter (−612 to +33) inserted into pGL3 reporter vector (pGL3/ATF2), 0.4 μg of c-jun promoter (−225 to +150, 49) and recloned in PGL3, luciferase containing vector 0.4 μg of c-jun promoter mutant (pGL3/c-jun^{MT}), containing mutated AP-1 site (50) both provided by Drs. McCance and Baglia (University of Rochester, Rochester, N.Y.), 0.4 μg of either empty pcDNA3 or the same vector containing hBVR (pcDNA3/hBVR), 0.4 μg of pCMVP-gal plasmid or 0.4 μg of empty pGL3 vector (without the inserts). Cells were harvested 48 h after transfection and lysed using Promega lysis buffer, the pellets were discarded by centrifuging for 5 min at 4° C. at maximum speed, and 5 μl of cell lysates were used for luciferase assays by using Promega kit (Madison, Wis.). Transfection efficiency was assessed by determining β-galactosidase activity, and luciferase activities were normalized by β-galactosodase activities.

### Example 11

### Verification of the Adenoviral Expression Vector for BVR Expression

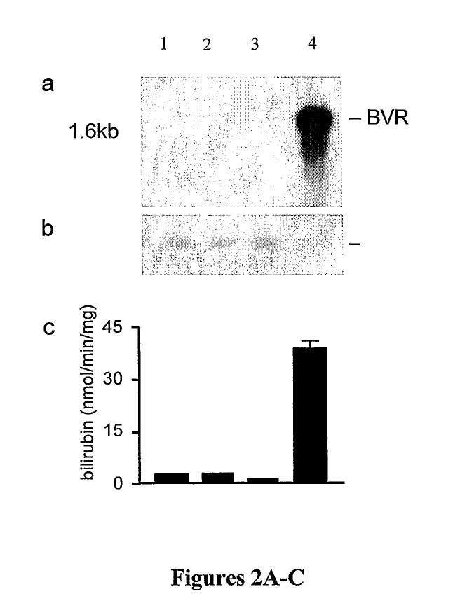

To investigate the potential of hBVR to alter gene expression in the cell, a two-component adenovirus system provided by BD Biosciences Clontech was used to develop an Ad-hBVR construct (FIG. 1). Functional hBVR cDNA was tagged with a FLAG sequence and cloned downstream in fusion with the EGFP gene taken from vector pEGFP-3C. The presence of FLAG and EGFP allowed for confirmation of BVR expression and cellular localization. The expressing cassette was subdloned into the shuttle vector pAdeno-X in two alternative orientations—one (named Ad-hBVR) under control of vector inducible promoter, the other (named AD-INV-hBVR) in the opposite orientation. The latter construct was designed to use in experiments as dominant-negative mutant of over-expressing clone Ad-hBVR. The helper virus Adeno-X Tet-On, the second component of the system, provides reverse tetracycline transactivator (rtTA), which in the presence of Dox binds to Tet-responsive element (TRE) located upstream of the minimal immediate early promoter of cytomegalovirus (P_{minCMV}) and subsequently activates transcription of the gene cloned under control of the promoter. This approach allowed to overexpress specific products of pAd-hBVR using regulated induction and to detect it either with anti-BVR or anti-FLAG antibodies or by EGFP fluorescence, which opens a broad range of opportunities such as study of hBVR trafficking (FIG. 2), protein-protein interactions, etc. In preliminary experiments, it was found that Dox in a concentration of 5 μg/mL provides the highest and most reproducible induction of recombinant protein (concentration of 0.01, 0.1, 0.5, 1.0, and 2.0 μg/ml were also tested).

Inducibility and expression of GFP-FLAG-hBVR mRNA was determined by Northern blot analysis (FIG. 2A). A prominent signal that corresponded to the size of the fusion product was observed 24 h after infection of 293A cells with Ad-hBVR. During the exposure period, exceedingly low levels of hBVR mRNA were detected in 293A cells, in cells infected with virus only, or in cells infected with Adeno-X Tet-On or Ad-INV-hBVR construct. The finding suggested that the increase is not due to activation of hBVR transcription by the virus. Robust increase in mRNA message of BVR was accompanied by a marked increase in rate of biliverdin reduction (FIG. 2C).

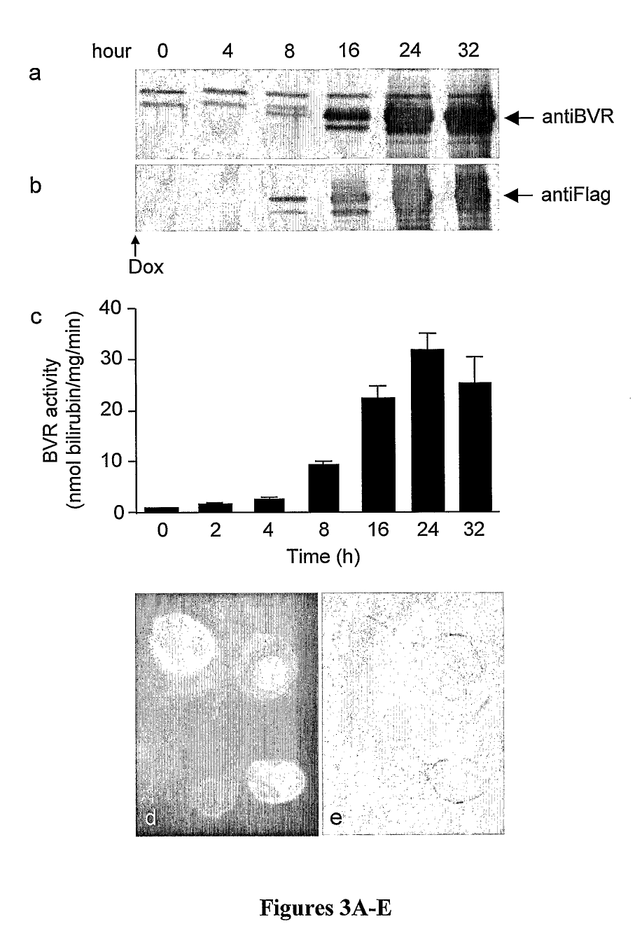

The time course of hBVR expression was examined. As shown in FIG. 3, there was a time dependent increase in protein yield (FIGS. 3A-B) and activity (FIG. 3C) in 293A cells transfected with hBVR. Prominent induction of hBVR was detected at the 8 h time point with anti-hBVR antibody. Western blot analysis using anti-BVR or anti-FLAG antibodies show practically identical patterns (FIG. 3A-B). It should be noted that endogenous BVR was detected with anti-BVR antibodies in non-induced 293 cells only when 20-fold higher amounts of cell extract was used for the analysis. Non-induced cells showed low level reductase activity; upon addition of Dox it was gradually increased following a general pattern as that of expression of BVR protein. The peak activity was noted at 24 h after the Dox induction. Similar to non-transfected cells, cells infected with virus only or infected with INV hBVR construct showed low level reductase activity. Cellular localization of hBVR was detected from EGFP green fluorescence. As shown in panel d, hBVR does traffic in the cells as indicated by the impressive EGFP green fluorescence of the nucleus. The hBVR was recently thought to be exclusively a cytosolic protein; the observed nuclear translocation of the protein is consistent with its function as a regulator of gene expression. Panel e shows cells visualized by visible light.

### Example 12

### Overexpression of BVR in 293 Cells Results in an Increase of ATF-2 Protein Expression

To test the genes that were affected by hBVR, total RNA was extracted from the 293A cells 24 h after infection either with Ad-hBVR or Ad-INV-hBVR viruses and subjected to gene micro array analysis of cell signaling genes. A number of genes were identified by the analysis. Only the genes with more the 2 fold changes in signal intensities are listed in the Table 1 (above); the brief description of their function is given in the table legend. The genes are representatives of such signal transduction pathways as Wnt pathway (c-JUN and WISP3), Hedgehog pathway (HHIP), TGF-b pathway (CDKN1B and CDKN2B), survival pathway (BCL2, BCL2L1, BIRC2, c-JUN), p53 pathway (GADD45), NF-kB pathway (PECAM1), CREB pathway (CYP19A1), Jak-Stat pathway (CSN2), estrogen pathway (EGFR, BCL2), calcium and protein kinase C pathway (PRKCA), phospholipase C pathway (PTGS2, BCL2, c-JUN), and stress pathway (ATF2, HSF1, HSPB1, HSPCA). Taking into account that hBVR is a potential transcription factor for AP-1 regulated genes (Ahmad et al., J. Biol. Chem. 277:9226-9232 (2002), which is hereby incorporated by reference in its entirety), and the members of the list of genes identified by microarray analysis are AP-1 regulated, whether their transcription could be directly effected by overexpressed hBVR protein was examined. The ATF2 gene, which contained three potential AP-1 binding sites in its promoter, was chosen for further studies.

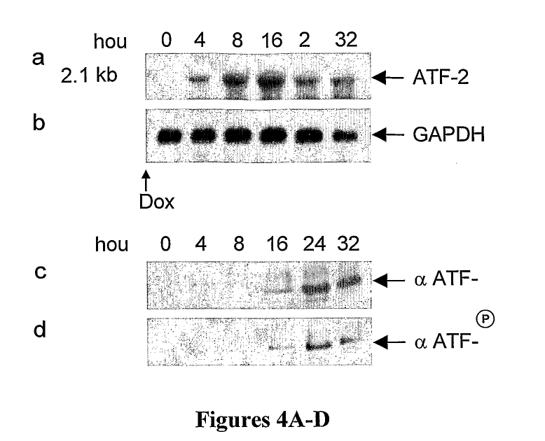

To confirm whether BVR in fact regulates ATF-2 mRNA and protein expression, 293 cells were infected with Ad-hBVR virus. Cells infected with the Ad-INV-hBVR were used as controls. The transfectants were harvested at different time points after antibiotic induction and used for Northern blot analysis of ATF-2 mRNA and ATF-2 protein by Western blot analysis. As shown in FIG. 4, ATF-2 mRNA was minimally detected in cells prior to the addition of the antibiotic. ATF-2 mRNA was increased with induction, and peak levels were detected at 16 h. The signal for GADPH, the control for loading, was essentially constant over the duration of the experiment. Infection with hBVR also resulted in a significant increase of ATF-2 protein expression (FIG. 4C). The increase was detectable 4h after induction and peaked at the 24 h time point. The DNA binding of ATF-2 is enhanced by N-terminal phosphorylation (Herdegen et al., Brain Res. Rev. 28:370-490 (1998), which is hereby incorporated by reference in its entirety). To test whether the phosphorylated form of ATF-2 was also increased by overexpression of BVR in the 293 cells, the expressed protein was probed with antibody to phospho thr69/71 ATF-2. As shown in FIG. 4D, an increase in the phosphorylated form of ATF-2 is detected 16 h after induction of antibiotic. The finding suggests that hBVR effects ATF-2 posttranslational modification. At this time, it is not evident whether the increase in phosphorylation of ATF-2 is the result of direct interaction of hBVR with ATF-2 or reflects modulation of other kinases that phosphorylate ATF-2. As shown in panel e, ATF-2 levels were not affected when cells were transfected with the inverted construct.

### Example 13

### hBVR Binds to ATF-2 Promoter

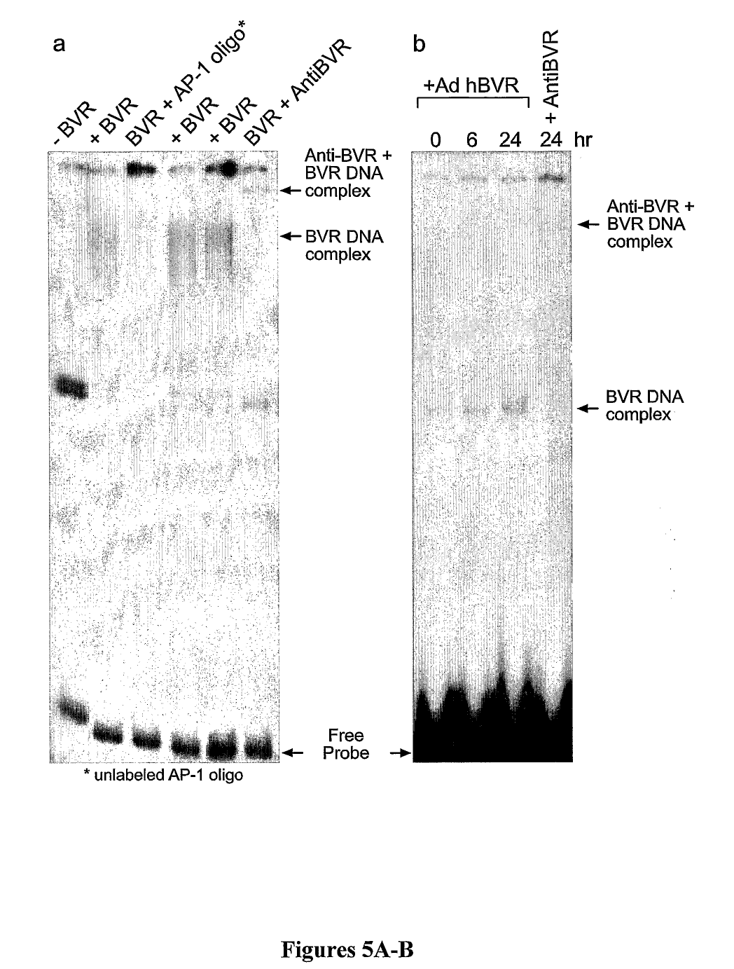

Because as overexpression of hBVR in 293A cells resulted in a significant increase in ATF-2 mRNA, whether hBVR interacts with consensus sequences in the ATF-2 promoter region was explored. Using Malinspector software, three potential AP-1 binding sites were predicted in 1 kb ATF-2 promoter region. A DNA fragment containing the three potential binding sites was extracted from the ATF-2 promoter by Hind III and NCOs I digestion. As shown in Table 3, one binding site has a similarity of 94% with AP-1 consensus sequence (TGTAGTCA), the other two have a similarity of 87% and 86%, respectively. hBVR was previously shown to bind to the AP-1 sites in the HO-1 promoter region (Ahmad et al., J. Biool. Clhem. 277:9226-9232 (2002), which is hereby incorporated by reference in its entirety). Presently, an AP-1 binding assay was carried out using hBVR translated in vitro using a TNT protein translation system and the ATF-2 promoter DNA fragment labeled with γ³²P-ATP. As shown in FIG. 5, in a gel mobility shift assay using 6% nondenatured polyacrylamide gel, a prominent protein+DNA signal was detected. To verify the specificity of hBVR-AP-1 site binding, unlabeled DNA was used for competition analyses. As seen in lane 3, FIG. 5, the intensity of the gel-shift band was nearly abolished when unlabeled DNA at the concentration of 10× excess of labeled DNA was present in the binding assay. Specificity of binding was further substantiated by the observation that adding hBVR specific antibody to the reaction mixture (lane 6) resulted in a super-shifted band of the findings suggesting that hBVR may be amongst the various factors that could effect ATF-2 transcription.

To further verify the specificity of BVR/AP-1 binding as detected by the gel shift assay using in vitro translated hBVR, nuclear extract was isolated from 239 cells transfected with Ad-BVR at times 0, 6 and 24 h after induction of hBVR expression and used for DNA binding assay. As shown in FIG. 5B, the gel shift assay revealed that overexpression of hBVR results in detectable increase of hBVR/AP-1 binding. After addition of anti-hBVR antibody, high mobility bands are detected. As noted here there are 2 super-shifted bands. These observations may well reflect the length of AP-1 probe, 300 bp, and the fact that this probe contains 3 potential AP-1 binding sites.

### Example 14

### BVR Binds to ATF/CRE Site

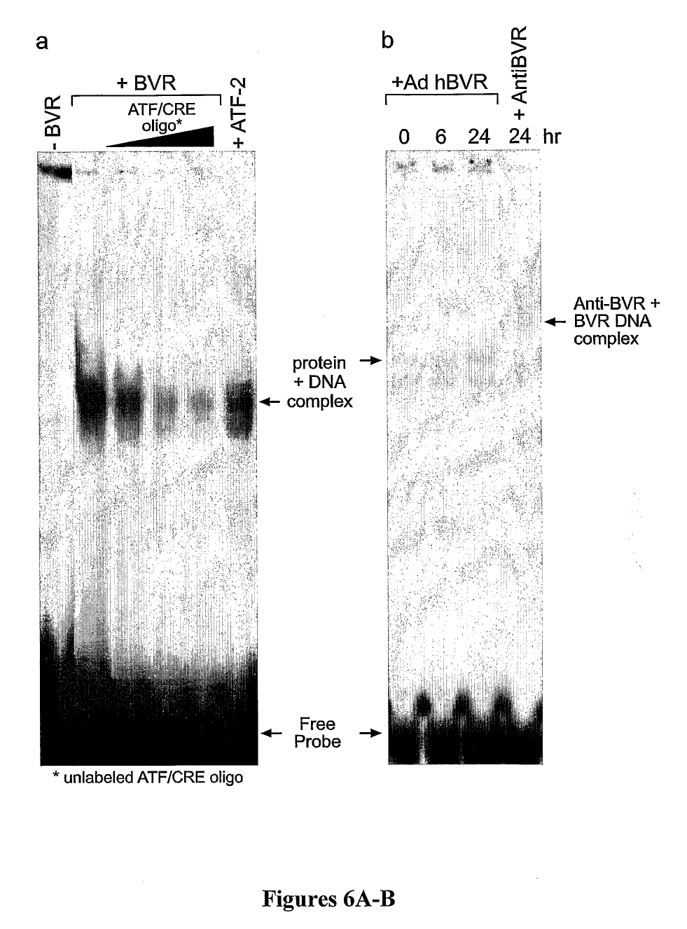

The consensus sequence of CRE, which is the binding site for dimeric ATF-2, differs from the AP-1 binding site by the presence of one added nucleotide to that of 7 nucleotide AP-1 binding sites (TGACNTCA vs. TGACTCA). Therefore, whether hBVR also binds to CRE was also explored. For this, gel mobility-shift assay was performed with in vitro translated hBVR protein and oligonucleotides containing CRE consensus sequence as listed in Tablel. As shown in FIG. 6, incubation of ³²-P labeled CRE oligo with translated BVR protein formed a complex that was detected by the gel mobility shift assay. To verify the sequence specificity of CRE binding, unlabeled oligonucleotide competition assay was performed. As shown in the Figure, the intensity of the gel-shift band was decreased in the presence of unlabeled CRE oligonucleotide. In the presence of unlabeled DNA at concentrations 2×, 5×, and 10× in excess of the labeled DNA, BVR-CRE binding was reduced by about 30%, 60% and 80%, respectively. Furthermore, the gel shift assay using nuclear extracts isolated from 239 cells transfected with Ad-BVR at times 0, 6 and 24 h after induction of hBVR expression shows significant increases of BVR/CRE binding and adding anti-BVR antibody into the DNA binding reaction mixture resulted in a super-shifted band (FIG. 6B). Furthermore, the gel shift assay using nuclear extracts isolated from 239 cells transfected with Ad-BVR at times 0, 6 and 24 h after induction of hBVR expression shows a significant increase of BVR/CRE binding and adding anti-BVR antibody into the DNA binding reaction mixture resulted in a super-shifted band (FIG. 6B). These results indicate that hBVR protein is capable of binding to CRE sites and suggests that hBVR like other DNA binding proteins, such as ATF-2, is capable of binding to structurally similar DNA sequences.

### Example 15

### BVR Upregulates Transcription of ATF-2 and c-jun

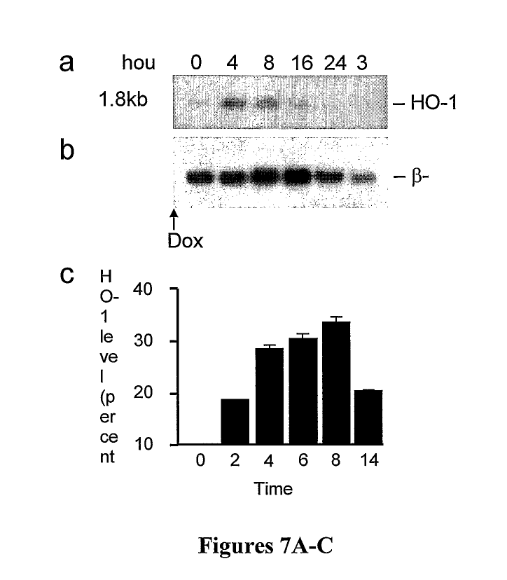

Transient transfection assays were performed to examine the effect of hBVR expression has on ATF-2 promoter directing CRE-mediated gene expression and c-jun promoter directing AP-1 mediated gene expression (FIG. 7). The sequence from −612 to +33 of ATF-2 promoter and −225 to +150 of c-jun promoter, a region that contains only 2 AP-1 binding sites (Clarke et al., Mol. Cell. Biol. 18:1065-1073 (1998), which is hereby incorporated by reference in its entirety) was inserted in the pGL3 vector containing luciferase reporter sequence and activity of the promoters was measured using luciferase assay kit (Promega, Madison, Wis.). As expected, ATF-2 and c-jun promoters alone produced several fold increase in luciferase activity. Whereas minimal luciferase activity was detected in cells transfected with pcDNA/hBVR alone or cotransfected with luciferase reporter empty vector with pcDNA3/hBVR. Cotransfection of the hBVR expression vector with either ATF-2 or c-jun promoter caused an additional 6-fold increase in luciferase activity. Luciferase activity was not similar to the basal promoter activity when cells were transfected with empty pcDNA3 vector. Furthermore, cotransfection of hBVR with reporter vector containing c-jun promoter reporter mutated at its TRRE site did not activate luciferase activity. This results in an unresponsive promoter (Han et al., Mol. Cell. Biol. 12:4472-4477 (1992), which is hereby incorporated by reference in its entirety). Taken together these observations support the suggestion made by Northern blotting and DNA gel shift assays that BVR is capable of inducing both ATF-2 and AP-1 responsive c-jun promoters.

### Example 16

### HO-1 Expression is Increased in hBVR Overexpressing Cells

The consequence of increased expression of hBVR on HO-1 was examined. Previous studies using antisense BVR suggested the presence of BVR is required for HO-1 stress response (Ahmad et al., J. Biol. Chem. 277:9226-9232 (2002), which is hereby incorporated by reference in its entirety), which requires AP-1 activation. As noted earlier, ATF-2 fonns a heterodimer with c-Jun with high affinity for AP-1 sites. Presently, the effects of increased BVR expression was examined to determine whether it would affect HO-1 expression. As shown in FIG. 7A, HO-1 mRNA in 293 cells was increased 8 h after induction. The observed increase in HO-1 mRNA did not result from differences in sample loading (FIG. 7C). Moreover, increase in HO-1 protein levels, as measured by ELISA, was consistent with an increase in HO-1 mRNA (FIG. 7C). The concentration of HO-1 in control cells was 46.7 ng/ml/mg of total cell proteins with that of Ad-IBVR infected cells measuring (238.9±8% of the control cells).

The presence of the bZip motif in the primary structure of hBVR, together with kinase activity of the reductase and its nuclear localization in response to oxidative stress and cGMP (Salim et al., J. Biol. Chem. 276:10929-10934 (2001); Maines et al., J. Pharmacol. Exper. Ther. 296:1091-1097 (2001); Ahmad et al., J. Biol. Chem. 277:9226-9232 (2002); which are hereby incorporated by reference in their entirety), is consistent with those of a protein that could potentially effect gene regulation. This concept was further reinforced by finding that hBVR could bind to a DNA fragment containing AP-1 sites and flanking nucleotides present in the HO-1 promoter (Alam et al., J. Biol. Che,. 267:21894-21900 (1992), which is hereby incorporated by reference in its entirety). It remained, however, to be established that in the cell hBVR has a role in regulation of gene expression. To explore the potential targets for hBVR in the cell, the protein was overexpressed using an adenovirus construct and altered gene expression was examined by microassay. This approach, namely overexpression of the target gene(s) in adenovirus vectors and microarray analysis is commonly used (Modur et al., J. Biol. Chem. 277:47928-47937 (2002); Sax et al., J. Biol. Chem. 278:36435-36444 (2003); Naiki et al., J. Biol. Chem. 277:14011-14019; Patel et al., J. Biol. Chem. 277:38915-38920 (2002); which are hereby incorporated by reference in their entirety).

The present investigation, which used a cell culture system, supports this potential and defines hBVR as a regulator of ATF-2 (CREB-2) and HO-1 expression. The AP-1 binding activity of hBVR was confirmed and was extended to the CRE site. CRE differs from the AP-1 site by one added base. Binding of hBVR to the consensus AP-1 and CRE sites in vitro, however, does not indicate that hBVR directly regulates ATF-2 or HO-1 transcription, rather this finding together with the results of mRNA and protein analysis support a role for hBVR in regulation of ATF-2 and HO-1 gene expression and potentially a component of the basal transcriptional machinery for their expression.

Increased levels of ATF-2 in cells overexpressing hBVR may affect a wide range of cellular functions. ATF-2 is a constitutive transcription factor whose expression, unlike that of c-Jun, which is an inducible factor, is not dependent on extracellular signals (Angel et al., Biochem. Biophys. Acta. 1072:129-157 (1991); Herdegen et al., Brain Res. Rev. 28-370-490 (1998), which are hereby incorporated by reference in their entirety). Transcriptional factors, Fos (Fos-like), c-Jun, and ATF-2, like BVR, are “leucine zipper” type factors and bind DNA in homo-dimeric or hetero-dimeric forms. The availability of the dimeric partner determines their preference for DNA binding sites. In the case of ATF-2, it can form a heterodimer with c-Jun. And, when its levels are increased, it effectively competes with c-Fos, the usual dimer partner of c-Jun. The ATF-2/c-Jun heterodimer preferentially binds to the seven base AP-1 sites (TGACTCA) rather than ATF-2's usual site, CRE (TGAC NTCA) (Ca/cAMP response element) (Herdegen et al., Brain Res. Rev. 28:370-490 (1998); Kawasaki et al., Nuc. Acids. Symp. Ser. 44:259-260 (2000); Lee et al., Diabetes 51:3400-3407 (2002), which are hereby incorporated by reference in their entirety). The ATF-2/c-Jun dimer DNA complex is more stable than c-Fos/c-Jun-DNA complex (Benbrook et al., Oncogene 5:295-302 (1990), which is hereby incorporated by reference in its entirety). Moreover, hetero-dimerization not only alters ATF-2 binding with remarkable variation in affinity amongst different CRE sites (Hagmneyer et al., EMBO J. 12:3559-3572 (1993), which is hereby incorporated by reference in its entirety), but also gene regulation activity of the dimeric partner. The ability of hBVR to induce ATF-2, therefore, is likely to change the profile of gene expression in the cell. In the case of heterodimerizing with c-Jun to form a component of AP-1 complex, the association likely will result in a wide spectrum of changes in the cell. AP-1 sites are found in promoter of a variety of genes (Angel et al., Cell 49:729-739 (1987), which is hereby incorporated by reference in its entirety) and is activated by mitogens, oncoproteins, cytokines, and stress inducing stimuli. ATF-2 in addition to influencing c-Jun's dimeric composition plays an important role in induction of c-jun gene expression and its autoregulatory transactivation by c-Jun protein.

ATF-2 also forms dimers with NF-Kβ (Du et al., Cell 74:887-898 (1993), which is hereby incorporated by reference in its entirety). NF-Kβ is a transcriptional activator of a number of proinflammatory mediators, such as cytokines, growth factors and adhesion molecules (Du et al., Cell 74:887-898 (1993); Peng et al., J. Biol. Chem. 270:17050-17055 (1995); which are hereby incorporated by reference in their entirety). It is reasonable to suggest that binding to enhancer elements of genes target two classes of NF-Kβ and their family of homo- or hetero-dimeric forms would be affected by an increase in ATF-2 in the cell. Activation by BVR of ATF-2, therefore, may be another mechanism by which NF-Kβ activity is modulated. Because NF-Kβ is a component of the signaling pathways that lead to conditions such as vascular inflammation and atherogenesis (Du et al., Cell 74:887-898 (1993); Peng et al., J. Biol. Chem. 270:17050-17055 (1995); which are hereby incorporated by reference in their entirety), the ability of BVR to increase ATF-2 gene expression should be useful in therapeutic settings to regulate inflammatory processes.

It is reasonable to suspect a relationship between increase in ATF-2 expression and increase in that of HO-1. Activation of MAPK signaling pathway and c-Jun/c-Fos-DNA binding is a key mechanism for the induction of HO-1. The oxygenase is also responsive to gene activation by cAMP (immenschuh et al., Biochem. J. 334:141-146 (1998); Durante et al., Amer. J. Physiol. 273:H317-H23; Pizurki et al., J. Cell. Physiol. 161:169-177 (1994); Nakagawa et al., J. Biol. Chem. 263:2460-2468 (1988); which are hereby incorporated by reference in their entirety). Accordingly, it is reasonable to suspect that induction of ATF-2 has a direct affect on HO-1 expression. However, the possibility cannot be ruled out that induced HO-1 gene expression in cells overexpressing hBVR, was independent of ATF-2. In this case, at the minimum, two mechanisms can be considered, one would involve the removal of the product, biliverdin, by hBVR functioning in the reductase capacity, and the other would involve activation of other genes that control HO-1 gene expression; for instance, that of c-jun. As noted in Table 1, c-jun was amongst the list of genes identified by micro array analyses to be upregulated in cells infected with Ad-hBVR. That all or a combination of the noted mechanisms are involved in upregulation of HO-1 is a distinct possibility. Considering the wide range of cellular functions that are modulated by HO activity and products of heme catalysis (Otterbein et al., Nat. Med. 6:422-428 (2000); Motterlini et al., Free Rad. Biol. Med. 28:1303-1312 (2000); Morse et al., Crit. Care. Med. 30:S12-S17 (2002); Zhang et al., J. Biol. Chem. 278:22061-22070 (2003); Chen et al., Exper. Biol. Med. 228:447-453 (2003); Otterbein et al., Trends Immunol. 24:449-455 (2003); Maines, Tox. Sci. 71:9-10 (2003); which are hereby incorporated by reference in their entirety), the finding that hBVR plays a role in regulation of HO-1 expression could be utilized as a method for modulating a wide spectrum of cellular processes.

Based on the findings of this investigation, it is reasonable to suggest that hBVR may play a role in regulation of these genes involved in proliferation, differentiation, survival and apoptosis.

### Example 17

### Construction of Retroviral Vectors Expressing siBVR

siBVR motifs according to the AA-N19 role were selected by finding the pattern in the human BVR cDNA sequence. The target sequence for hBVR is located at position 100 bases downstream of the start codon (nt 100-nt 121). A retroviral based vector pSuper-Retro for expressing siRNA was purchased from OligoEngine Co. (Seattle, Wash., USA). siRNA expressing vector pSuper-siBVR was constructed according manufacturer's instruction using the oligomers described below. Briefly, oligos (SEQ ID NOS: 17 and 18, respectively) containing the sequence of 21-mer small interference RNA (italicized) were synthesized as follows:

GATCCCCTCCTCAGCGTTCCTGAACCTGTTCAAGAGACAGGTTCAGGAAC
GCTGAGGATTTTTGGAAAGGGAGGAGTCGCAAGGACTTGGACAAGTTCTC
TGTCCAAGTCCTTGCGACTCCTAAAAACCTTTTCGA

SEQ ID NO: 17 (upper sequence) appears in 5′-3′ orientation, whereas SEQ ID NO: 18 (lower sequence) appears in the 3′-5′ orientation. The pSuper-siBVR expression vector expressed the double-stranded RNA hairpin, which upon cleavage afforded the siRNA duplex containing the sequence(s) appearing in italics.

### Example 18

### siRNA Mediated Knock-Down of BVR Increased Sensitivity of 293 Cells to Arsenite Toxicity

To evaluate if BVR and HO-1 effectively protect the cells from external stress such as arsenite treatment, flow-cytometry based analysis of apoptosis of the cells was employed. 293 cells were treated with 100 uM of arsenite for 8 hours and the cells were subsequently harvested. After washing, the cells were fixed with 75% ethanol and stained with 2OuM of propidium iodide (PI) with 2 g/ml of RNAse A. The cells were further analyzed by FACS for apoptosis based on the PI staining. The results showed that siBVR mediated knock-down resulted in 50% more apoptotic cells compared with control, on average. The data indicates that BVR is an effective protector of cells from oxidative stress caused by arsenite.

Although the invention has been described in detail for the purpose of illustration, it is understood that such detail is solely for that purpose, and variations can be made therein by those skilled in the art without departing from the spirit and scope of the invention which is defined by the following claims.

Each of the references cited in the present application is intended to be incorporated herein in its entirety by reference to the same.

What is claimed:

1. A method of modifying expression of cell cycle or cell signaling proteins comprising:
modifying the nuclear or cellular concentration of biliverdin reductase, or fragments or variants thereof, in a cell, whereby a change in the nuclear or cellular concentration of biliverdin reductase, or fragments or variants thereof, modifies the transcription or expression of cell cycle or cell signaling proteins.

2. The method according to claim 1 wherein said modifying comprises:
transforming the cell with a DNA construct which expresses antisense biliverdin reductase RNA or siRNA in the cell, said transforming decreasing the nuclear or cellular concentration of biliverdin reductase.

3. The method according to claim 1 wherein said modifying comprises:
transforming the cell with a DNA construct which expresses biliverdin reductase or fragments or variants thereof in the cell, said transforming increasing the nuclear or cellular concentration of biliverdin reductase or fragments or variants thereof.

4. The method according to claim 1 wherein said modifying comprises:
introducing biliverdin reductase or fragments or variants thereof into the cell.

5. The method according to claim 4 wherein said introducing comprises:
contacting the cell with a delivery vehicle comprising biliverdin reductase or fragments or variants thereof under conditions effective to induce cellular uptake of at least the biliverdin reductase or fragments or variants thereof.

6. The method according to claim 5 wherein the delivery vehicle is a liposome comprising biliverdin reductase or fragments or variants thereof.

7. The method according to claim 5 wherein the delivery vehicle is a fusion protein comprising biliverdin reductase or fragments or variants thereof.

8. The method according to claim 1 wherein the cell is ex vivo.

9. The method according to claim 1 wherein the cell is in vivo.

10. The method according to claim 1 wherein the cell signal protein is selected from the group consisting of creb-2, bax, bfl-1, IAP-1, IAP-2, p16Ink4, beta-casein, p450XIX, GADD45, HIP, p27Kip1, p15Ink2b, p18 (cdk4 inhibitor), CDXl, FASN, Stra6.

11. The method according to claim 1 wherein the cell cycling protein is selected from the group consisting of cyclins A, E1 and E2, CDK15a, CDC7, cdk1, cdk2, cdk8, Cks2, Cks1p9, Cul1, Cul2, Cul3, E2F-3, MAD2L1, MCM6, Rbx1, RAD50, cdk4, CDK10, and RPL13A.

12. A method of treating a condition associated with an expression levels of a cell cycle or a cell signaling protein, said method comprising:
performing the method according to claim 1 in a cell, thereby altering the expression of a cell cycle or cell signaling protein to treat a condition associated therewith.

13. The method according to claim 12 wherein said performing achieves a reduction in cellular or nuclear concentration of biliverdin reductase.

14. The method according to claim 12 wherein said performing achieves an increase in cellular or nuclear concentration of biliverdin reductase.

15. The method according to claim 12 wherein the condition is associated with a cell signaling protein.

16. The method according to claim 15 wherein the cell signaling protein is selected from the group of creb-2, bax, bfl-1, IAP-1, IAP-2, p16Ink4, beta-casein, p450XIX, GADD45, HIP, p27Kip1, p15Ink2b, p18 (cdk4 inhibitor), CDX1, FASN, Stra6.

17. The method according to claim 15 wherein the cell signaling protein is creb-2.

18. The method according to claim 17 wherein the creb-2 associated condition is selected from the group of stress-related cell growth and/or cell proliferation and NF-Kβ mediated vascular inflammation or atherogenesis.

19. The method according to claim 1 wherein the condition is associated with a cell cycling protein.

20. The method according to claim 19 wherein the cell cycling protein is selected from the group consisting of cyclins A, E1 and E2, CDK15a, CDC7, cdk1, cdk2, cdk8, Cks2, Cks1p9, Cul1, Cul2, Cul3, E2F-3, MAD2L1, MCM6, Rbx1, RAD50, cdk4, CDK10, and RPL13A.

21. A DNA molecule encoding an siRNA that inhibits biliverdin reductase.

22. The DNA molecule according to claim 21 comprising the nucleotide sequence of nt 8-58 from SEQ ID NO: 17.

23. An expression vector comprising the DNA molecule according to claim 21.

24. A host cell comprising the DNA molecule according to claim 21.

25. The host cell according to claim 24 wherein the host cell is ex vivo.

26. The host cell according to claim 24 wherein the host cell is in vivo.

27. A siRNA molecule encoded by the DNA molecule according to claim 21, wherein the siRNA molecule comprises less than about 30 nucleotides and inhibits biliverdin reductase expression.

28. The siRNA molecule according to claim 27 wherein the RNA nucleotide sequence comprises SEQ ID NO: 8.

29. The siRNA molecule according to claim 27 wherein the siRNA molecule is in the forn of an RNA duplex.


SEQUENCE LISTING (7 sequences)

SEQ ID NO: 1 - skipped

SEQ ID NO: 2 - DNA, 1070 bp, Human BVR
ggggtggcgcccggagctgcacggagagcgtgcccgtcagtgaccgaagaagagaccaagatgaatgcagagcccgagaggaagtttggcgtggtggtggttggtgttggccgagccggctccgtgcggatgagggacttgcggaatccacacccttcctcagcgttcctgaacctgattggcttcgtgtcgagaagggagctcgggagcattgatggagtccagcagatttctttggaggatgctctttccagccaagaggtggaggtcgcctatatctgcagtgagagctccagccatgaggactacatcaggcagttccttaatgctggcaagcacgtccttgtggaataccccatgacactgtcattggcggccgctcaggaactgtgggagctggctgagcagaaaggaaaagtcttgcacgaggagcatgttgaactcttgatggaggaattcgctttcctgaaaaaagaagtggtggggaaagacctgctgaaagggtcgctcctcttcacatctgacccgttggaagaagaccggtttggcttccctgcattcagcggcatctctcgactgacctggctggtctccctctttggggagctttctcttgtgtctgccactttggaagagcgaaaggaagatcagtatatgaaaatgacagtgtgtctggagacagagaagaaaagtccactgtcatggattgaagaaaaaggacctggtctaaaacgaaacagatatttaagcttccatttcaagtctgggtccttggagaatgtgccaaatgtaggagtgaataagaacatatttctgaaagatcaaaatatatttgtccagaaactcttgggccagttctctgagaaggaactggctgctgaaaagaaacgcatcctgcactgcctggggcttgcagaagaaatccagaaatattgctgttcaaggaagtaagaggaggaggtgatgtagcacttccaagatggcaccagcatttggttcttctcaagagttgaccattatctctattcttaaaattaaacatgttggggaaacaaaaaaaaaaaaaaaaa

SEQ ID NO: 3 - protein, 296 aa, Human BVR
MNTEPERKFGVVVVGVGRAGSVRMRDLRNPHPSSAFLNLIGFVSRRELGSIDGVQQISLEDALSSQEVEVAYICSESSSHEDYIRQFLNAGKHVLVEYPMTLSLAAAQELWELAEQKGKVLHEEHVELLMEEFAFLKKEVVGKDLLKGSLLFTAGPLEEERFGFPAFSGISRLTWLVSLFGELSLVSATLEERKEDQYMKMTVCLETEKKSPLSWIEEKGPGLKRNRYLSFHFKSGSLENVPNVGVNKNIFLKDQNIFVQKLLGQFSEKELAAEKKRILHCLGLAEEIQKYCCSRK

SEQ ID NO: 4 - protein, 295 aa, Rat BVR
MDAEPKRKFGVVVVGVGRAGSVRLRDLKDPRSAAFLNLIGFVSRRELGSLDEVRQISLEDALRSQEIDVAYICSESSSHEDYIRQFLQAGKHVLVEYPMTLSFAAAQELWELAAQKGRVLHEEHVELLMEEFEFLRREVLGKELLKGSLRFTASPLEEERFGFPAFSGISRLTWLVSLFGELSLISATLEERKEDQYMKMTVQLETQNKGLLSWIEEKGPGLKRNRYVNFQFTSGSLEEVPSVGVNKNIFLKDQDIFVQKLLDQVSAEDLAAEKKRIMHCLGLASDIQKLCHQKK

SEQ ID NO: 5 - DNA, 1081 bp, Rat BVR
ggtcaacagctaagtgaagccatatccatagagagtttgtgccagtgccccaagatcctgaacctctgtctgtcttcggacactgactgaagagaccgagatggatgccgagccaaagaggaaatttggagtggtagtggttggtgttggcagagctggctcggtgaggctgagggacttgaaggatccacgctctgcagcattcctgaacctgattggatttgtgtccagacgagagcttgggagccttgatgaagtacggcagatttctttggaagatgctctccgaagccaagagattgatgtcgcctatatttgcagtgagagttccagccatgaagactatatacggcagtttctgcaggctggcaagcatgtcctcgtggaataccccatgacactgtcatttgcggcggcccaggagctgtgggagctggccgcacagaaagggagagtcctgcatgaggagcacgtggaactcttgatggaggaattcgaattcctgagaagagaagtgttggggaaagagctactgaaagggtctcttcgcttcacagctagcccactggaagaagagagatttggcttccctgcgttcagcggcatttctcgcctgacctggctggtctccctcttcggggagctttctcttatttctgccaccttggaagagcgaaaagaggatcagtatatgaaaatgaccgtgcagctggagacccagaacaagggtctgctgtcatggattgaagagaaagggcctggcttaaaaagaaacagatatgtaaacttccagttcacttctgggtccctggaggaagtgccaagtgtaggggtcaataagaacattttcctgaaagatcaggatatatttgttcagaagctcttagaccaggtctctgcagaggacctggctgctgagaagaagcgcatcatgcattgcctggggctggccagcgacatccagaagctttgccaccagaagaagtgaagaggaagcttcagagacttctgaagggggccagggtttggtcctatcaaccattcacctttagctcttacaattaaacatgtcagataaaca

SEQ ID NO: 6 - protein, 295 aa, Mouse BVR
MSTEPKRKFGVVVVGVGRAGSVRIRDSKDPHSSAFLNLIGYVSRRELGSLDNVRQISLEDALRSQEVDVAYICTESSSHEDYIRQFLQAGKHVLVEYPMALSFAAAQELWELAAQKGRVLHEEHIELLMEEFEFLKREVAGKELLKGSLRFTASPLEEEKFGFPAFSGISRLTWLVSLFGELSLISATMENRKEDQYMKMTVQLETQNKSPLSWIEEKGPGLKRNRHISIHFKSGSLEEVPNVGVNKNIFLKDQDIFIQKLLGQVSAEDLAAEKKRILHCLELASDIQRLCHRKQ

SEQ ID NO: 7 - protein, 296 aa, Pig BVR
MNAEPERKFGVVVVGVGRAGSVRMRDLRNPHPSSAFLNLIGFVSRRELGSIDGVQQISLEDALSSQEVEVAYICSESSSHEDYIRQFLNAGKHVLVEYPMTLSLAAAQELWELAEQKGKVLHEEHVELLMEEFAFLKKEVVGKDLLKGSLLFTAGPLEEERFGSPAFSGISRLTWLVSLFGELSLVSATLEERKEDQYMKMTVCLETEKKSPLSWIEEKGPGLKRNRYLSFHFKSGSLENVPNVGVNKNIFLKDQNIFVQKLLGQFSEKELAAEKKRILHCLGLAEEIQKYCCSRK